SEQUENCE LISTING (46 sequences)

SEQ ID NO: 1 - DNA, 22 bp, Artificial Sequence
  pp65 forward primer
atcaaaccgggcaagatctcgc

SEQ ID NO: 2 - DNA, 22 bp, Artificial Sequence
  pp65 reverse primer
atcgtactgacgcagttccacg

SEQ ID NO: 3 - DNA, 20 bp, Artificial Sequence
  IE1 exon4 forward primer
ccatcgccgaggagtcagat

SEQ ID NO: 4 - DNA, 20 bp, Artificial Sequence
  IE1 exon4 reverse primer
agtgtcctcccgctcctcct

SEQ ID NO: 5 - DNA, 20 bp, Artificial Sequence
  IEfusion forward  primer
aagttgccccagaggaagag

SEQ ID NO: 6 - DNA, 20 bp, Artificial Sequence
  IEfusion reverse primer
ctgctaacgctgcaagagtg

SEQ ID NO: 7 - DNA, 21 bp, Artificial Sequence
  TK forward primer
tgtgagcgtatggcaaacgaa

SEQ ID NO: 8 - DNA, 28 bp, Artificial Sequence
  TK reverse primer
tcgatgtaacactttctacacaccgatt

SEQ ID NO: 9 - DNA, 9388 bp, Artificial Sequence
  mH5-IEfusion-pZWIIA (GUS) plasmid DNA sequence
cctcctgaaaaactggaatttaatacaccatttgtgttcatcatcagacatgatattactggatttatattgtttatgggtaaggtagaatctccttaatatgggtacggtgtaaggaatcattattttatttatattgatgggtacgtgaaatctgaattttcttaataaatattatttttattaaatgtgtatatgttgttttgcgatagccatgtatctactaatcagatctattagagatattattaattctggtgcaatatgacaaaaattatacactaattagcgtctcgtttcagacatggatctgtcacgaattaatacttggaagtctaagcagctgaaaagctttctctctagcaaagatgcatttaaggcggatgtccatggacatagtgccttgtattatgcaatagctgataataacgtgcgtctagtatgtacgttgttgaacgctggagcattgaaaaatcttctagagaatgaatttccattacatcaggcagccacattggaagataccaaaatagtaaagattttggctattcagtggactggatgattcgaggtaccgactattgttctatattatatatggttgttgatggatctgtgatgcatgcaatagctgataatagaacttacgcaaatattagcaaaaatatattagacaatactacaattaacgatgagtgtagatgctgttattttgaaccacagattaggattcttgatagagatgagatgctcaatggatcatcgtgtgatatgaacagacattgtattatgatgaatttacctgatgtaggcgaatttggatctagtatgttggggaaatatgaacctgacatgattaagattgctctttcggtggctgggtaccaggcgcgcatttcattttgtttttttctatgctataaatggtacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgagagctcggttgttgatggatctgtgatgcatgcaatagctgataatagaacttacgcaaatattagcaaaaatatattagacaatactacaattaacgatgagtgtagatgctgttattttgaaccacagattaggattcttgatagagatgagatgctcaatggatcatcgtgtgatatgaacagacattgtattatgatgaatttacctgatgtaggcgaatttggatctagtatgttggggaaatatgaacctgacatgattaagattgctctttcggtggctggcggcccgctcgagaaaaattgaaaataaatacaaaggttcttgagggttgtgttaaattgaaagcgagaaataatcataaataagccaccaccgtttaaacgccaccacaatggtcaaacagattaaggttcgagtggacatggtgcggcatagaatcaaggagcacatgctgaaaaaatatacccagacggaagagaaattcactggcgcctttaatatgatgggaggatgtttgcagaatgccttagatatcttagataaggttcatgagcctttcgaggagatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcactgcagaacttgcctcagtgctcccctgatgagattatggcttatgcccagaaaatatttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctgggggccgatcctctgagagtctgctctcctagtgtggatgacctacgggccatcgccgaggagtcagatgaggaagaggctattgtagcctacactttggccaccgctggtgtcagctcctctgattctctggtgtcacccccagagtcccctgtacccgcgactatccctctgtcctcagtaattgtggctgagaacagtgatcaggaagaaagtgagcagagtgatgaggaagaggaggagggtgctcaggaggagcgggaggacactgtgtctgtcaagtctgagccagtgtctgagatagaggaagttgccccagaggaagaggaggatggtgctgaggaacccaccgcctctggaggcaagagcacccaccctatggtgactagaagcaaggctgaccagggtgacatcctcgcccaggctgtcaatcatgccggtatcgattccagtagcaccggccccacgctgacaacccactcttgcagcgttagcagcgcccctcttaacaagccgacccccaccagcgtcgcggttactaacactcctctccccggggcatccgctactcccgagctcagcccgcgtaagaaaccgcgcaaaaccacgcgtcctttcaaggtgattattaaaccgcccgtgcctcccgcgcctatcatgctgcccctcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttccccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggttttggtggcgcggcctcctcctctctgctgagctgcggccatcagagcagcggcggggcgagcaccggaccccgcaagaagaagagcaaacgcatctccgagttggacaacgagaaggtgcgcaatatcatgaaagataagaacacccccttctgcacacccaacgtgcagactcggcggggtcgcgtcaagattgacgaggtgagccgcatgttccgcaacaccaatcgctctcttgagtacaagaacctgcccttcacgattcccagtatgcaccaggtgttagatgaggccatcaaagcctgcaaaaccatgcaggtgaacaacaagggcatccagattatctacacccgcaatcatgaggtgaagagtgaggtggatgcggtgcggtgtcgcctgggcaccatgtgcaacctggccctctccactcccttcctcatggagcacaccatgcccgtgacacatccacccgaagtggcgcagcgcacagccgatgcttgtaacgaaggcgtcaaggccgcgtggagcctcaaagaattgcacacccaccaattatgcccccgttcctccgattaccgcaacatgatcatccacgctgccacccccgtggacctgttgggcgctctcaacctgtgcctgcccctgatgcaaaagtttcccaaacaggtcatggtgcgcatcttctccaccaaccagggtgggttcatgctgcctatctacgagacggccgcgaaggcctacgccgtggggcagtttgagcagcccaccgagacccctcccgaagacctggacaccctgagcctggccatcgaggcagccatccaggacctgaggaacaagtctcagtaaaataaaggcgcgccataaaaatttttatactagtgtaccgcggtcgaatcgatttaattaacgatgctagcattgtcgacggtggtggcgcggccgccagtgtgatggatatctgcagaattcggcttggggggctgcaggtggatgcgatcatgacgtcctctgcaatggataacaatgaacctaaagtactagaaatggtatatgatgctacaattttacccgaaggtagtagcatggattgtataaacagacacatcaatatgtgtatacaacgcacctatagttctagtataattgccatattggatagattcctaatgatgaacaaggatgaactaaataatacacagtgtcatataattaaagaatttatgacatacgaacaaatggcgattgaccattatggagaatatgtaaacgctattctatatcaaattcgtaaaagacctaatcaacatcacaccattaatctgtttaaaaaaataaaaagaacccggtatgacacttttaaagtggatcccgtagaattcgtaaaaaaagttatcggatttgtatctatcttgaacaaatataaaccggtttatagttacgtcctgtacgagaacgtcctgtacgatgagttcaaatgtttcattgactacgtggaaactaagtatttctaaaattaatgatgcattaatttttgtattgattctcaatcctaaaaactaaaatatgaataagtattaaacatagcggtgtactaattgatttaacataaaaaatagttgttaactaatcatgaggactctacttattagatatattctttggagaaatgacaacgatcaaaccgggcatgcaagcttgtctccctatagtgagtcgtattagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttcgagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggatttaggtgacactatagaatacgaattc

SEQ ID NO: 10 - DNA, 8152 bp, Artificial Sequence
  mH5-pp65-GUS-pLW51(GUS) plasmid DNA sequence
gaattcgttggtggtcgccatggatggtgttattgtatactgtctaaacgcgttagtaaaacatggcgaggaaataaatcatataaaaaatgatttcatgattaaaccatgttgtgaaaaagtcaagaacgttcacattggcggacaatctaaaaacaatacagtgattgcagatttgccatatatggataatgcggtatccgatgtatgcaattcactgtataaaaagaatgtatcaagaatatccagatttgctaatttgataaagatagatgacgatgacaagactcctactggtgtatataattattttaaacctaaagatgccattcctgttattatatccataggaaaggatagagatgtttgtgaactattaatctcatctgataaagcgtgtgcgtgtatagagttaaattcatataaagtagccattcttcccatggatgtttccttttttaccaaaggaaatgcatcattgattattctcctgtttgatttctctatcgatgcggcacctctcttaagaagtgtaaccgataataatgttattatatctagacaccagcgtctacatgacgagcttccgagttccaattggttcaagttttacataagtataaagtccgactattgttctatattatatatggttgttgatggatctgtgatgcatgcaatagctgataatagaacttacgcaaatattagcaaaaatatattagacaatactacaattaacgatgagtgtagatgctgttattttgaaccacagattaggattcttgatagagatgagatgctcaatggatcatcgtgtgatatgaacagacattgtattatgatgaatttacctgatgtaggcgaatttggatctagtatgttggggaaatatgaacctgacatgattaagattgctctttcggtggctgggtaccaggcgcgcatttcattttgtttttttctatgctataaatggtacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgagagctcggttgttgatggatctgtgatgcatgcaatagctgataatagaacttacgcaaatattagcaaaaatatattagacaatactacaattaacgatgagtgtagatgctgttattttgaaccacagattaggattcttgatagagatgagatgctcaatggatcatcgtgtgatatgaacagacattgtattatgatgaatttacctgatgtaggcgaatttggatctagtatgttggggaaatatgaacctgacatgattaagattgctctttcggtggctggcggcccgctcgagaaaaattgaaaataaatacaaaggttcttgagggttgtgttaaattgaaagcgagaaataatcataaataagccaccaccgtttaaacagtcgacggtatcgataagcttgatatcgaattcctgcagcccgtacgcgcaggcagcatggagtcgcgcggtcgccgttgtcccgaaatgatatccgtactgggtcccatttcggggcacgtgctgaaagccgtgtttagtcgcggcgacacgccggtgctgccgcacgagacgcgactcctgcagacgggtatccacgtgcgcgtgagccagccctcgctgatcctggtgtcgcagtacacgcccgactcgacgccatgccaccgcggcgacaatcagctgcaggtgcagcacacgtactttacgggcagcgaggtggagaacgtgtcggtcaacgtgcacaaccccacgggccggagcatctgccccagccaagagcccatgtcgatctatgtgtacgcgctgccgctcaagatgctgaacatccccagcatcaacgtgcaccactacccgtcggcggccgagcgcaaacaccgacacctgcccgtagctgacgctgtgattcacgcgtcgggcaagcagatgtggcaggcgcgtctcacggtctcgggactggcctggacgcgtcagcagaaccagtggaaagagcccgacgtctactacacgtcagcgttcgtgtttcccaccaaggacgtggcactgcggcacgtggtgtgcgcgcacgagctggtttgctccatggagaacacgcgcgcaaccaagatgcaggtgataggtgaccagtacgtcaaggtgtacctggagtccttctgcgaggacgtgccctccggcaagctctttatgcacgtcacgctgggctctgacgtggaagaggacctgacgatgacccgcaacccgcaacccttcatgcgcccccacgagcgcaacggctttacggtgttgtgtcccaaaaatatgataatcaaaccgggcaagatctcgcacatcatgctggatgtggcttttacctcacacgagcattttgggctgctgtgtcccaagagcatcccgggcctgagcatctcaggtaacctattgatgaacgggcagcagatcttcctggaggtgcaagcgatacgcgagaccgtggaactgcgtcagtacgatcccgtggctgcgctcttctttttcgatatcgacttgctgctgcagcgcgggcctcagtacagcgaacaccccaccttcaccagccagtatcgcatccagggcaagcttgagtaccgacacacctgggaccggcacgacgagggtgccgcccagggcgacgacgacgtctggaccagcggatcggactccgacgaggaactcgtaaccaccgagcgcaagacgccccgcgttaccggcggcggcgccatggcgggcgcctccacttccgcgggccgcaaacgcaaatcagcatcctcggcgacggcgtgcacggcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaacccaaacgtcgccgccaccggcaagacgccttgcccgggccatgcatcgcctcgacgcccaaaaagcaccgaggttgatttttatggcgcgccctgcagggaaagttttataggtagttgatagaacaaaatacataattttgtaaaaataaatcactttttatactaatatgacacgattaccaatacttttgttactaatatcattagtatacgctacaccttttcctcagacatctaaaaaaataggtgatgatgcaactttatcatgtaatcgaaataatacaaatgactacgttgttatgagtgcttggtataaggagcccaattccattattcttttagctgctaaaagcgacgtcttgtattttgataattataccaaggataaaatatcttacgactctccatacgatgatctagttacaactatcacaattaaatcattgactgctagagatgccggtacttatgtatgtgcattctttatgacatcgcctacaaatgacactgataaagtagattatgaagaatactccacagagttgattgtaaatacagatagtgaatcgactatagacataatactatctggatctacacattcaccagaaactagttaagcttgtctccctatagtgagtcgtattagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttcgagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggatttaggtgacactata

SEQ ID NO: 11 - DNA, 2709 bp, Artificial Sequence
  HCMV IEfusion gene
atggtcaaacagattaaggttcgagtggacatggtgcggcatagaatcaaggagcacatgctgaaaaaatatacccagacggaagagaaattcactggcgcctttaatatgatgggaggatgtttgcagaatgccttagatatcttagataaggttcatgagcctttcgaggagatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcactgcagaacttgcctcagtgctcccctgatgagattatggcttatgcccagaaaatatttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctgggggccgatcctctgagagtctgctctcctagtgtggatgacctacgggccatcgccgaggagtcagatgaggaagaggctattgtagcctacactttggccaccgctggtgtcagctcctctgattctctggtgtcacccccagagtcccctgtacccgcgactatccctctgtcctcagtaattgtggctgagaacagtgatcaggaagaaagtgagcagagtgatgaggaagaggaggagggtgctcaggaggagcgggaggacactgtgtctgtcaagtctgagccagtgtctgagatagaggaagttgccccagaggaagaggaggatggtgctgaggaacccaccgcctctggaggcaagagcacccaccctatggtgactagaagcaaggctgaccagggtgacatcctcgcccaggctgtcaatcatgccggtatcgattccagtagcaccggccccacgctgacaacccactcttgcagcgttagcagcgcccctcttaacaagccgacccccaccagcgtcgcggttactaacactcctctccccggggcatccgctactcccgagctcagcccgcgtaagaaaccgcgcaaaaccacgcgtcctttcaaggtgattattaaaccgcccgtgcctcccgcgcctatcatgctgcccctcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttccccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggttttggtggcgcggcctcctcctctctgctgagctgcggccatcagagcagcggcggggcgagcaccggaccccgcaagaagaagagcaaacgcatctccgagttggacaacgagaaggtgcgcaatatcatgaaagataagaacacccccttctgcacacccaacgtgcagactcggcggggtcgcgtcaagattgacgaggtgagccgcatgttccgcaacaccaatcgctctcttgagtacaagaacctgcccttcacgattcccagtatgcaccaggtgttagatgaggccatcaaagcctgcaaaaccatgcaggtgaacaacaagggcatccagattatctacacccgcaatcatgaggtgaagagtgaggtggatgcggtgcggtgtcgcctgggcaccatgtgcaacctggccctctccactcccttcctcatggagcacaccatgcccgtgacacatccacccgaagtggcgcagcgcacagccgatgcttgtaacgaaggcgtcaaggccgcgtggagcctcaaagaattgcacacccaccaattatgcccccgttcctccgattaccgcaacatgatcatccacgctgccacccccgtggacctgttgggcgctctcaacctgtgcctgcccctgatgcaaaagtttcccaaacaggtcatggtgcgcatcttctccaccaaccagggtgggttcatgctgcctatctacgagacggccgcgaaggcctacgccgtggggcagtttgagcagcccaccgagacccctcccgaagacctggacaccctgagcctggccatcgaggcagccatccaggacctgaggaacaagtctcagtaa

SEQ ID NO: 12 - DNA, 46 bp, Artificial Sequence
  Primer “a” for construction of the IEfusion gene
agctttgtttaaacgccaccaccatggtcaaacagattaaggttcg

SEQ ID NO: 13 - DNA, 60 bp, Artificial Sequence
  Primer “b” for construction of the IEfusion gene
ggcatgattgacagcctgggcgaggatgtcaccctggtcagccttgcttctagtcaccat

SEQ ID NO: 14 - DNA, 69 bp, Artificial Sequence
  Primer “c” for construction of the IEfusion gene
tgttagcgtgggcccggtgctactggaatcgataccggcatgattgacagcctgggcgaggatgtcacc

SEQ ID NO: 15 - DNA, 28 bp, Artificial Sequence
  Primer “d” for construction of the IEfusion gene
tagcaccgggcccacgctaacaacccac

SEQ ID NO: 16 - DNA, 39 bp, Artificial Sequence
  Primer “e” for construction of the IEfusion gene
ttggcgcgcctttattttactgagacttgttcctcaggt

SEQ ID NO: 17 - protein, 9 aa, Artificial Sequence
  carboxyl terminus of the 580 amino acid primary sequence of the IE2 protein [Swiss Prot #P19893]
GPEDQNTSC

SEQ ID NO: 18 - protein, 17 aa, Artificial Sequence
  E1/e4-IE2/e5 (IEfusion) gene
MVTRSKADQGDILAQAV

SEQ ID NO: 19 - DNA, 2709 bp, Artificial Sequence
  Synthetic polynucleotide IEfusion-VacO
atggtgaagcaaatcaaggtcagagtggacatggtaagacacagaattaaggaacacatgttgaagaagtatactcaaacagaggagaagttcaccggtgccttcaatatgatgggtggatgtctacagaacgctttggatatcttagataaggtacatgaaccattcgaagaaatgaagtgcattggattgacaatgcaatcaatgtatgagaactacatagtgccagaggataagcgtgaaatgtggatggcatgcatcaaggagttacatgatgtatccaaaggagcagccaacaagctaggtggtgctttgcaagcgaaggcaagagcgaagaaggatgaattgagacgaaagatgatgtacatgtgctatcgaaacatcgaattcttcactaagaactcagcgtttcctaagactaccaatggatgcagtcaagctatggctgcgcttcagaacttgcctcaatgtagtcctgatgaaatcatggcatatgcacagaagatcttcaagatcttagatgaggaaagagacaaggtattgactcatatcgatcacatattcatggatatactaacaacatgtgtagaaacgatgtgtaacgagtacaaggtaacttcggacgcttgtatgatgactatgtacggaggaatatctctacttagtgagttctgtcgagttctatgctgttacgtattagaagaaactagtgtaatgttagcgaagagaccattgatcactaagcctgaagtgatctcggttatgaagagacgaatagaggagatctgtatgaaggtgttcgcacaatacatcttaggagctgatcctctaagagtgtgtagtccatcggtagacgatttgagagctatagcggaggaatctgacgaggaagaggcaatagttgcatacacacttgctacagctggagtatccagttctgattctcttgtaagtcctccggagtcacctgtgccagcaaccataccgttgagtagtgtgattgtggctgagaactcggatcaggaagagtctgagcaatccgatgaagaagaggaggaaggagcacaagaggagagagaagatactgtctctgtgaagagtgaacctgtatctgaaatcgaggaagtagcacctgaggaagaggaggatggagccgaagaaccaacagcttcgggtggtaagtcaactcatccgatggtaaccagatctaaggcagaccagggagacatcctagcacaagcagtgaaccatgctggaattgactcatcttcgaccggaccaactctaacgactcattcatgttcggttagttctgctcctcttaacaagcctacacctacctcggtagctgttaccaacacacctttaccaggagcatcagcaacacctgagttgtctccaagaaagaagcctcgtaagaccacgagaccgttcaaggtgatcatcaagccaccagtaccacctgctccgatcatgttgccattgatcaagcaggaggacattaagccagaacctgacttcacgatacagtaccgtaacaagatcatagatacagcaggatgcatagtgatctcagatagtgaagaggagcaaggtgaggaagtggagactagaggagccacagccagttcgccttccacaggatccggaactcctagagtaactagtccgacacatccactttcccagatgaatcatccacctctaccggatcctctaggacgaccagatgaagattcttcttcatctagttcaagttcttgctcatccgcgagtgatagtgagtcagaaagtgaagagatgaagtgctcttctggtggtggagctagtgtcacttcatctcatcatggacgaggaggatttggaggtgctgcgagtagttccttactaagttgtggacatcagtcatctggtggtgcatctactggacctagaaagaagaagtcaaagagaatctccgaattggataatgagaaagtgagaaacatcatgaaggacaagaacacgccgttctgcactccgaatgttcagacgagaagaggacgagtgaagatagatgaagtatcacgaatgttcagaaacacaaatcgttctctagagtacaagaatcttccgttcaccataccttcgatgcaccaagtattagatgaggctatcaaggcatgtaagaccatgcaagttaacaacaaaggaatacagatcatctacactagaaaccatgaggttaagagtgaggtggatgccgtacgttgtagattgggaacgatgtgtaaccttgcgctatctactcctttcctaatggagcatactatgcctgtgactcatcctcctgaagtggctcaaagaacagctgatgcttgtaacgaaggtgtgaaagctgcttggtccctaaaggagttacatacacaccaactttgtccacgatccagtgactacagaaacatgatcattcatgcagctacgcctgtagatctacttggagctcttaacctatgtcttcctttgatgcagaagttccctaagcaagtgatggtgagaatcttctcgacgaatcaaggaggattcatgttaccgatatacgagacagctgcaaaggcttacgctgtcggtcagttcgagcaaccgactgaaacgcctcctgaggacttagatacattgtctttggcgatagaagcagcgattcaggatcttagaaacaagagtcagtaa

SEQ ID NO: 20 - DNA, 2709 bp, Artificial Sequence
  Synthetic polynucleotide IEfusion-4nt
atggtcaaacagattaaggttcgagtggacatggtgcggcatagaatcaaggagcacatgctgaagaagtatacccagacggaagagaaattcactggcgcctttaatatgatgggaggatgtttgcagaatgccttagatatcttagataaggttcatgagcctttcgaggagatgaagtgtattgggctaactatgcagagcatgtatgagaactacattgtacctgaggataagcgggagatgtggatggcttgtattaaggagctgcatgatgtgagcaagggcgccgctaacaagttaggaggtgcactgcaggctaaggcccgtgctaagaaggatgaacttaggagaaagatgatgtatatgtgctacaggaatatagagttctttaccaagaactcagccttccctaagaccaccaatggctgcagtcaggccatggcggcactgcagaacttgcctcagtgctctcctgatgagattatggcttatgcccagaagatatttaagatcttggatgaggagagagacaaggtgctcacgcacattgatcacatatttatggatatcctcactacatgtgtggaaacaatgtgtaatgagtacaaggtcactagtgacgcttgtatgatgaccatgtacggaggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctaggtgccgatcctctgagagtctgctctcctagtgtggatgacctacgggccatcgccgaggagtcagatgaggaagaggctattgtagcctacactttggccaccgctggtgtcagctcctctgattctctggtgtcacctccagagtcacctgtacccgcgactatccctctgtcctcagtaattgtggctgagaacagtgatcaggaagaaagtgagcagagtgatgaggaagaggaggagggtgctcaggaggagcgggaggacactgtgtctgtcaagtctgagccagtgtctgagatagaggaagttgctccagaggaagaggaggatggtgctgaggaacccaccgcctctggaggcaagagcacccaccctatggtgactagaagcaaggctgaccagggtgacatcctcgcccaggctgtcaatcatgccggtatcgattccagtagcaccggacctacgctgacaacccactcttgcagcgttagcagcgctcctcttaacaagccgactccaaccagcgtcgcggttactaacactcctctaccaggagcatccgctactcccgagctcagcccgcgtaagaaaccgcgcaagaccacgcgtcctttcaaggtgattattaaaccgcccgtgcctcccgcgcctatcatgctgccactcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttcaccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggatttggtggcgcggcctcctcctctctgctgagctgcggccatcagagcagcggcggtgcgagcaccggacctcgcaagaagaagagcaaacgcatctccgagttggacaacgagaaggtgcgcaatatcatgaaagataagaacactcccttctgcacacccaacgtgcagactcggcgtggtcgcgtcaagattgacgaggtgagccgcatgttccgcaacaccaatcgctctcttgagtacaagaacctgcccttcacgattcccagtatgcaccaggtgttagatgaggccatcaaagcctgcaagaccatgcaggtgaacaacaagggcatccagattatctacacccgcaatcatgaggtgaagagtgaggtggatgcggtgcggtgtcgcctgggcaccatgtgcaacctggccctctccactcccttcctcatggagcacaccatgcccgtgacacatccacccgaagtggcgcagcgcacagccgatgcttgtaacgaaggcgtcaaggccgcgtggagcctcaaagaattgcacacccaccaattatgtcctcgttcctccgattaccgcaacatgatcatccacgctgccacaccagtggacctgttgggcgctctcaacctgtgcctgccactgatgcagaagtttcccaaacaggtcatggtgcgcatcttctccaccaaccagggtgggttcatgctgcctatctacgagacggccgcgaaggcctacgccgttggtcagtttgagcagcccaccgagacacctcccgaagacctggacaccctgagcctggccatcgaggcagccatccaggacctgaggaacaagtctcagtaa

SEQ ID NO: 21 - protein, 902 aa, Artificial Sequence
  Synthetic polypeptide IEfusion-Vac0
MVKQIKVRVDMVRHRIKEHMLKKYTQTEEKFTGAFNMMGGCLQNALDILDKVHEPFEEMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMAYAQKIFKILDEERDKVLTHIDHIFMDILTTCVETMCNEYKVTSDACMMTMYGGISLLSEFCRVLCCYVLEETSVMLAKRPLITKPEVISVMKRRIEEICMKVFAQYILGADPLRVCSPSVDDLRAIAEESDEEEAIVAYTLATAGVSSSDSLVSPPESPVPATIPLSSVIVAENSDQEESEQSDEEEEEGAQEEREDTVSVKSEPVSEIEEVAPEEEEDGAEEPTASGGKSTHPMVTRSKADQGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEHTMPVTHPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 22 - protein, 902 aa, Artificial Sequence; identical to SEQ ID NO: 21
  Synthetic polypeptide IEfusion

SEQ ID NO: 23 - protein, 902 aa, Artificial Sequence; identical to SEQ ID NO: 21
  Synthetic polypeptide IEfusion-4nt

SEQ ID NO: 24 - DNA, 1491 bp, Artificial Sequence
  Synthetic polynucleotide IE2-Vac0
atgggagacatcctagcacaagcagtgaaccatgctggaattgactcatcttcgaccggaccaactctaacgactcattcatgttcggttagttctgctcctcttaacaagcctacacctacctcggtagctgttaccaacacacctttaccaggagcatcagcaacacctgagttgtctccaagaaagaagcctcgtaagaccacgagaccgttcaaggtgatcatcaagccaccagtaccacctgctccgatcatgttgccattgatcaagcaggaggacattaagccagaacctgacttcacgatacagtaccgtaacaagatcatagatacagcaggatgcatagtgatctcagatagtgaagaggagcaaggtgaggaagtggagactagaggagccacagccagttcgccttccacaggatccggaactcctagagtaactagtccgacacatccactttcccagatgaatcatccacctctaccggatcctctaggacgaccagatgaagattcttcttcatctagttcaagttcttgctcatccgcgagtgatagtgagtcagaaagtgaagagatgaagtgctcttctggtggtggagctagtgtcacttcatctcatcatggacgaggaggatttggaggtgctgcgagtagttccttactaagttgtggacatcagtcatctggtggtgcatctactggacctagaaagaagaagtcaaagagaatctccgaattggataatgagaaagtgagaaacatcatgaaggacaagaacacgccgttctgcactccgaatgttcagacgagaagaggacgagtgaagatagatgaagtatcacgaatgttcagaaacacaaatcgttctctagagtacaagaatcttccgttcaccataccttcgatgcaccaagtattagatgaggctatcaaggcatgtaagaccatgcaagttaacaacaaaggaatacagatcatctacactagaaaccatgaggttaagagtgaggtggatgccgtacgttgtagattgggaacgatgtgtaaccttgcgctatctactcctttcctaatggagcatactatgcctgtgactcatcctcctgaagtggctcaaagaacagctgatgcttgtaacgaaggtgtgaaagctgcttggtccctaaaggagttacatacacaccaactttgtccacgatccagtgactacagaaacatgatcattcatgcagctacgcctgtagatctacttggagctcttaacctatgtcttcctttgatgcagaagttccctaagcaagtgatggtgagaatcttctcgacgaatcaaggaggattcatgttaccgatatacgagacagctgcaaaggcttacgctgtcggtcagttcgagcaaccgactgaaacgcctcctgaggacttagatacattgtctttggcgatagaagcagcgattcaggatcttagaaacaagagtcagtaa

SEQ ID NO: 25 - DNA, 1491 bp, Artificial Sequence
  Synthetic polynucleotide IE2
atgggtgacatcctcgcccaggctgtcaatcatgccggtatcgattccagtagcaccggccccacgctgacaacccactcttgcagcgttagcagcgcccctcttaacaagccgacccccaccagcgtcgcggttactaacactcctctccccggggcatccgctactcccgagctcagcccgcgtaagaaaccgcgcaaaaccacgcgtcctttcaaggtgattattaaaccgcccgtgcctcccgcgcctatcatgctgcccctcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttccccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggttttggtggcgcggcctcctcctctctgctgagctgcggccatcagagcagcggcggggcgagcaccggaccccgcaagaagaagagcaaacgcatctccgagttggacaacgagaaggtgcgcaatatcatgaaagataagaacacccccttctgcacacccaacgtgcagactcggcggggtcgcgtcaagattgacgaggtgagccgcatgttccgcaacaccaatcgctctcttgagtacaagaacctgcccttcacgattcccagtatgcaccaggtgttagatgaggccatcaaagcctgcaaaaccatgcaggtgaacaacaagggcatccagattatctacacccgcaatcatgaggtgaagagtgaggtggatgcggtgcggtgtcgcctgggcaccatgtgcaacctggccctctccactcccttcctcatggagcacaccatgcccgtgacacatccacccgaagtggcgcagcgcacagccgatgcttgtaacgaaggcgtcaaggccgcgtggagcctcaaagaattgcacacccaccaattatgcccccgttcctccgattaccgcaacatgatcatccacgctgccacccccgtggacctgttgggcgctctcaacctgtgcctgcccctgatgcaaaagtttcccaaacaggtcatggtgcgcatcttctccaccaaccagggtgggttcatgctgcctatctacgagacggccgcgaaggcctacgccgtggggcagtttgagcagcccaccgagacccctcccgaagacctggacaccctgagcctggccatcgaggcagccatccaggacctgaggaacaagtctcagtaa

SEQ ID NO: 26 - DNA, 1491 bp, Artificial Sequence
  Synthetic polynucleotide IE2-4nt
atgggtgacatcctcgcccaggctgtcaatcatgccggtatcgattccagtagcaccggacctacgctgacaacccactcttgcagcgttagcagcgctcctcttaacaagccgactccaaccagcgtcgcggttactaacactcctctaccaggagcatccgctactcccgagctcagcccgcgtaagaaaccgcgcaagaccacgcgtcctttcaaggtgattattaaaccgcccgtgcctcccgcgcctatcatgctgccactcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttcaccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggatttggtggcgcggcctcctcctctctgctgagctgcggccatcagagcagcggcggtgcgagcaccggacctcgcaagaagaagagcaaacgcatctccgagttggacaacgagaaggtgcgcaatatcatgaaagataagaacactcccttctgcacacccaacgtgcagactcggcgtggtcgcgtcaagattgacgaggtgagccgcatgttccgcaacaccaatcgctctcttgagtacaagaacctgcccttcacgattcccagtatgcaccaggtgttagatgaggccatcaaagcctgcaagaccatgcaggtgaacaacaagggcatccagattatctacacccgcaatcatgaggtgaagagtgaggtggatgcggtgcggtgtcgcctgggcaccatgtgcaacctggccctctccactcccttcctcatggagcacaccatgcccgtgacacatccacccgaagtggcgcagcgcacagccgatgcttgtaacgaaggcgtcaaggccgcgtggagcctcaaagaattgcacacccaccaattatgtcctcgttcctccgattaccgcaacatgatcatccacgctgccacaccagtggacctgttgggcgctctcaacctgtgcctgccactgatgcagaagtttcccaaacaggtcatggtgcgcatcttctccaccaaccagggtgggttcatgctgcctatctacgagacggccgcgaaggcctacgccgttggtcagtttgagcagcccaccgagacacctcccgaagacctggacaccctgagcctggccatcgaggcagccatccaggacctgaggaacaagtctcagtaa

SEQ ID NO: 27 - DNA, 1491 bp, Artificial Sequence
  Synthetic polynucleotide IE2 4nt H363A
atgggtgacatcctcgcccaggctgtcaatcatgccggtatcgattccagtagcaccggacctacgctgacaacccactcttgcagcgttagcagcgctcctcttaacaagccgactccaaccagcgtcgcggttactaacactcctctaccaggagcatccgctactcccgagctcagcccgcgtaagaaaccgcgcaagaccacgcgtcctttcaaggtgattattaaaccgcccgtgcctcccgcgcctatcatgctgccactcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttcaccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggatttggtggcgcggcctcctcctctctgctgagctgcggccatcagagcagcggcggtgcgagcaccggacctcgcaagaagaagagcaaacgcatctccgagttggacaacgagaaggtgcgcaatatcatgaaagataagaacactcccttctgcacacccaacgtgcagactcggcgtggtcgcgtcaagattgacgaggtgagccgcatgttccgcaacaccaatcgctctcttgagtacaagaacctgcccttcacgattcccagtatgcaccaggtgttagatgaggccatcaaagcctgcaagaccatgcaggtgaacaacaagggcatccagattatctacacccgcaatcatgaggtgaagagtgaggtggatgcggtgcggtgtcgcctgggcaccatgtgcaacctggccctctccactcccttcctcatggaggcaaccatgcccgtgacacatccacccgaagtggcgcagcgcacagccgatgcttgtaacgaaggcgtcaaggccgcgtggagcctcaaagaattgcacacccaccaattatgtcctcgttcctccgattaccgcaacatgatcatccacgctgccacaccagtggacctgttgggcgctctcaacctgtgcctgccactgatgcagaagtttcccaaacaggtcatggtgcgcatcttctccaccaaccagggtgggttcatgctgcctatctacgagacggccgcgaaggcctacgccgttggtcagtttgagcagcccaccgagacacctcccgaagacctggacaccctgagcctggccatcgaggcagccatccaggacctgaggaacaagtctcagtaa

SEQ ID NO: 28 - DNA, 1491 bp, Artificial Sequence
  Synthetic polynucleotide IE2 4nt H369A
atgggtgacatcctcgcccaggctgtcaatcatgccggtatcgattccagtagcaccggacctacgctgacaacccactcttgcagcgttagcagcgctcctcttaacaagccgactccaaccagcgtcgcggttactaacactcctctaccaggagcatccgctactcccgagctcagcccgcgtaagaaaccgcgcaagaccacgcgtcctttcaaggtgattattaaaccgcccgtgcctcccgcgcctatcatgctgccactcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttcaccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggatttggtggcgcggcctcctcctctctgctgagctgcggccatcagagcagcggcggtgcgagcaccggacctcgcaagaagaagagcaaacgcatctccgagttggacaacgagaaggtgcgcaatatcatgaaagataagaacactcccttctgcacacccaacgtgcagactcggcgtggtcgcgtcaagattgacgaggtgagccgcatgttccgcaacaccaatcgctctcttgagtacaagaacctgcccttcacgattcccagtatgcaccaggtgttagatgaggccatcaaagcctgcaagaccatgcaggtgaacaacaagggcatccagattatctacacccgcaatcatgaggtgaagagtgaggtggatgcggtgcggtgtcgcctgggcaccatgtgcaacctggccctctccactcccttcctcatggagcacaccatgcccgtgacagcaccacccgaagtggcgcagcgcacagccgatgcttgtaacgaaggcgtcaaggccgcgtggagcctcaaagaattgcacacccaccaattatgtcctcgttcctccgattaccgcaacatgatcatccacgctgccacaccagtggacctgttgggcgctctcaacctgtgcctgccactgatgcagaagtttcccaaacaggtcatggtgcgcatcttctccaccaaccagggtgggttcatgctgcctatctacgagacggccgcgaaggcctacgccgttggtcagtttgagcagcccaccgagacacctcccgaagacctggacaccctgagcctggccatcgaggcagccatccaggacctgaggaacaagtctcagtaa

SEQ ID NO: 29 - DNA, 1491 bp, Artificial Sequence
  Synthetic polynucleotide IE2 4nt H363A/H369A
atgggtgacatcctcgcccaggctgtcaatcatgccggtatcgattccagtagcaccggacctacgctgacaacccactcttgcagcgttagcagcgctcctcttaacaagccgactccaaccagcgtcgcggttactaacactcctctaccaggagcatccgctactcccgagctcagcccgcgtaagaaaccgcgcaagaccacgcgtcctttcaaggtgattattaaaccgcccgtgcctcccgcgcctatcatgctgccactcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttcaccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggatttggtggcgcggcctcctcctctctgctgagctgcggccatcagagcagcggcggtgcgagcaccggacctcgcaagaagaagagcaaacgcatctccgagttggacaacgagaaggtgcgcaatatcatgaaagataagaacactcccttctgcacacccaacgtgcagactcggcgtggtcgcgtcaagattgacgaggtgagccgcatgttccgcaacaccaatcgctctcttgagtacaagaacctgcccttcacgattcccagtatgcaccaggtgttagatgaggccatcaaagcctgcaagaccatgcaggtgaacaacaagggcatccagattatctacacccgcaatcatgaggtgaagagtgaggtggatgcggtgcggtgtcgcctgggcaccatgtgcaacctggccctctccactcccttcctcatggaggcaaccatgcccgtgacagcaccacccgaagtggcgcagcgcacagccgatgcttgtaacgaaggcgtcaaggccgcgtggagcctcaaagaattgcacacccaccaattatgtcctcgttcctccgattaccgcaacatgatcatccacgctgccacaccagtggacctgttgggcgctctcaacctgtgcctgccactgatgcagaagtttcccaaacaggtcatggtgcgcatcttctccaccaaccagggtgggttcatgctgcctatctacgagacggccgcgaaggcctacgccgttggtcagtttgagcagcccaccgagacacctcccgaagacctggacaccctgagcctggccatcgaggcagccatccaggacctgaggaacaagtctcagtaa

SEQ ID NO: 30 - DNA, 1488 bp, Artificial Sequence
  Synthetic polynucleotide IE2 H363A
atgggtgacatcctcgcccaggctgtcaatcatgccggtatcgattccagtagcaccggccccacgctgacaacccactcttgcagcgttagcagcgcccctcttaacaagccgacccccaccagcgtcgcggttactaacactcctctccccggggcatccgctactcccgagctcagcccgcgtaagaaaccgcgcaaaaccacgcgtcctttcaaggtgattattaaaccgcccgtgcctcccgcgcctatcatgctgcccctcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttccccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggttttggtggcgcggcctcctcctctctgctgagctgcggccatcagagcagcggcggggcgagcaccggaccccgcaagaagaagagcaaacgcatctccgagttggacaacgagaaggtgcgcaatatcatgaaagataagaacacccccttctgcacacccaacgtgcagactcggcggggtcgcgtcaagattgacgaggtgagccgcatgttccgcaacaccaatcgctctcttgagtacaagaacctgcccttcacgattcccagtatgcaccaggtgttagatgaggccatcaaagcctgcaaaaccatgcaggtgaacaacaagggcatccagattatctacacccgcaatcatgaggtgaagagtgaggtggatgcggtgcggtgtcgcctgggcaccatgtgcaacctggccctctccactcccttcctcatggaggcaaccatgcccgtgacacatccacccgaagtggcgcagcgcacagccgatgcttgtaacgaaggcgtcaaggccgcgtggagcctcaaagaattgcacacccaccaattatgcccccgttcctccgattaccgcaacatgatcatccacgctgccacccccgtggacctgttgggcgctctcaacctgtgcctgcccctgatgcaaaagtttcccaaacaggtcatggtgcgcatcttctccaccaaccagggtgggttcatgctgcctatctacgagacggccgcgaaggcctacgccgtggggcagtttgagcagcccaccgagacccctcccgaagacctggacaccctgagcctggccatcgaggcagccatccaggacctgaggaacaagtctcag

SEQ ID NO: 31 - DNA, 1488 bp, Artificial Sequence
  Synthetic polynucleotide IE2 H369A
atgggtgacatcctcgcccaggctgtcaatcatgccggtatcgattccagtagcaccggccccacgctgacaacccactcttgcagcgttagcagcgcccctcttaacaagccgacccccaccagcgtcgcggttactaacactcctctccccggggcatccgctactcccgagctcagcccgcgtaagaaaccgcgcaaaaccacgcgtcctttcaaggtgattattaaaccgcccgtgcctcccgcgcctatcatgctgcccctcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttccccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggttttggtggcgcggcctcctcctctctgctgagctgcggccatcagagcagcggcggggcgagcaccggaccccgcaagaagaagagcaaacgcatctccgagttggacaacgagaaggtgcgcaatatcatgaaagataagaacacccccttctgcacacccaacgtgcagactcggcggggtcgcgtcaagattgacgaggtgagccgcatgttccgcaacaccaatcgctctcttgagtacaagaacctgcccttcacgattcccagtatgcaccaggtgttagatgaggccatcaaagcctgcaaaaccatgcaggtgaacaacaagggcatccagattatctacacccgcaatcatgaggtgaagagtgaggtggatgcggtgcggtgtcgcctgggcaccatgtgcaacctggccctctccactcccttcctcatggagcacaccatgcccgtgacagcaccacccgaagtggcgcagcgcacagccgatgcttgtaacgaaggcgtcaaggccgcgtggagcctcaaagaattgcacacccaccaattatgcccccgttcctccgattaccgcaacatgatcatccacgctgccacccccgtggacctgttgggcgctctcaacctgtgcctgcccctgatgcaaaagtttcccaaacaggtcatggtgcgcatcttctccaccaaccagggtgggttcatgctgcctatctacgagacggccgcgaaggcctacgccgtggggcagtttgagcagcccaccgagacccctcccgaagacctggacaccctgagcctggccatcgaggcagccatccaggacctgaggaacaagtctcag

SEQ ID NO: 32 - DNA, 1488 bp, Artificial Sequence
  Synthetic polynucleotide IE2 H363A/H369A
atgggtgacatcctcgcccaggctgtcaatcatgccggtatcgattccagtagcaccggccccacgctgacaacccactcttgcagcgttagcagcgcccctcttaacaagccgacccccaccagcgtcgcggttactaacactcctctccccggggcatccgctactcccgagctcagcccgcgtaagaaaccgcgcaaaaccacgcgtcctttcaaggtgattattaaaccgcccgtgcctcccgcgcctatcatgctgcccctcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttccccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggttttggtggcgcggcctcctcctctctgctgagctgcggccatcagagcagcggcggggcgagcaccggaccccgcaagaagaagagcaaacgcatctccgagttggacaacgagaaggtgcgcaatatcatgaaagataagaacacccccttctgcacacccaacgtgcagactcggcggggtcgcgtcaagattgacgaggtgagccgcatgttccgcaacaccaatcgctctcttgagtacaagaacctgcccttcacgattcccagtatgcaccaggtgttagatgaggccatcaaagcctgcaaaaccatgcaggtgaacaacaagggcatccagattatctacacccgcaatcatgaggtgaagagtgaggtggatgcggtgcggtgtcgcctgggcaccatgtgcaacctggccctctccactcccttcctcatggaggcaaccatgcccgtgacagcaccacccgaagtggcgcagcgcacagccgatgcttgtaacgaaggcgtcaaggccgcgtggagcctcaaagaattgcacacccaccaattatgcccccgttcctccgattaccgcaacatgatcatccacgctgccacccccgtggacctgttgggcgctctcaacctgtgcctgcccctgatgcaaaagtttcccaaacaggtcatggtgcgcatcttctccaccaaccagggtgggttcatgctgcctatctacgagacggccgcgaaggcctacgccgtggggcagtttgagcagcccaccgagacccctcccgaagacctggacaccctgagcctggccatcgaggcagccatccaggacctgaggaacaagtctcag

SEQ ID NO: 33 - DNA, 1488 bp, Artificial Sequence
  Synthetic polynucleotide IE2 VacO H363A
atgggagacatcctagcacaagcagtgaaccatgctggaattgactcatcttcgaccggaccaactctaacgactcattcatgttcggttagttctgctcctcttaacaagcctacacctacctcggtagctgttaccaacacacctttaccaggagcatcagcaacacctgagttgtctccaagaaagaagcctcgtaagaccacgagaccgttcaaggtgatcatcaagccaccagtaccacctgctccgatcatgttgccattgatcaagcaggaggacattaagccagaacctgacttcacgatacagtaccgtaacaagatcatagatacagcaggatgcatagtgatctcagatagtgaagaggagcaaggtgaggaagtggagactagaggagccacagccagttcgccttccacaggatccggaactcctagagtaactagtccgacacatccactttcccagatgaatcatccacctctaccggatcctctaggacgaccagatgaagattcttcttcatctagttcaagttcttgctcatccgcgagtgatagtgagtcagaaagtgaagagatgaagtgctcttctggtggtggagctagtgtcacttcatctcatcatggacgaggaggatttggaggtgctgcgagtagttccttactaagttgtggacatcagtcatctggtggtgcatctactggacctagaaagaagaagtcaaagagaatctccgaattggataatgagaaagtgagaaacatcatgaaggacaagaacacgccgttctgcactccgaatgttcagacgagaagaggacgagtgaagatagatgaagtatcacgaatgttcagaaacacaaatcgttctctagagtacaagaatcttccgttcaccataccttcgatgcaccaagtattagatgaggctatcaaggcatgtaagaccatgcaagttaacaacaaaggaatacagatcatctacactagaaaccatgaggttaagagtgaggtggatgccgtacgttgtagattgggaacgatgtgtaaccttgcgctatctactcctttcctaatggaggctactatgcctgtgactcatcctcctgaagtggctcaaagaacagctgatgcttgtaacgaaggtgtgaaagctgcttggtccctaaaggagttacatacacaccaactttgtccacgatccagtgactacagaaacatgatcattcatgcagctacgcctgtagatctacttggagctcttaacctatgtcttcctttgatgcagaagttccctaagcaagtgatggtgagaatcttctcgacgaatcaaggaggattcatgttaccgatatacgagacagctgcaaaggcttacgctgtcggtcagttcgagcaaccgactgaaacgcctcctgaggacttagatacattgtctttggcgatagaagcagcgattcaggatcttagaaacaagagtcag

SEQ ID NO: 34 - DNA, 1488 bp, Artificial Sequence
  Synthetic polynucleotide IE2 VacO H369A
atgggagacatcctagcacaagcagtgaaccatgctggaattgactcatcttcgaccggaccaactctaacgactcattcatgttcggttagttctgctcctcttaacaagcctacacctacctcggtagctgttaccaacacacctttaccaggagcatcagcaacacctgagttgtctccaagaaagaagcctcgtaagaccacgagaccgttcaaggtgatcatcaagccaccagtaccacctgctccgatcatgttgccattgatcaagcaggaggacattaagccagaacctgacttcacgatacagtaccgtaacaagatcatagatacagcaggatgcatagtgatctcagatagtgaagaggagcaaggtgaggaagtggagactagaggagccacagccagttcgccttccacaggatccggaactcctagagtaactagtccgacacatccactttcccagatgaatcatccacctctaccggatcctctaggacgaccagatgaagattcttcttcatctagttcaagttcttgctcatccgcgagtgatagtgagtcagaaagtgaagagatgaagtgctcttctggtggtggagctagtgtcacttcatctcatcatggacgaggaggatttggaggtgctgcgagtagttccttactaagttgtggacatcagtcatctggtggtgcatctactggacctagaaagaagaagtcaaagagaatctccgaattggataatgagaaagtgagaaacatcatgaaggacaagaacacgccgttctgcactccgaatgttcagacgagaagaggacgagtgaagatagatgaagtatcacgaatgttcagaaacacaaatcgttctctagagtacaagaatcttccgttcaccataccttcgatgcaccaagtattagatgaggctatcaaggcatgtaagaccatgcaagttaacaacaaaggaatacagatcatctacactagaaaccatgaggttaagagtgaggtggatgccgtacgttgtagattgggaacgatgtgtaaccttgcgctatctactcctttcctaatggagcatactatgcctgtgactgctcctcctgaagtggctcaaagaacagctgatgcttgtaacgaaggtgtgaaagctgcttggtccctaaaggagttacatacacaccaactttgtccacgatccagtgactacagaaacatgatcattcatgcagctacgcctgtagatctacttggagctcttaacctatgtcttcctttgatgcagaagttccctaagcaagtgatggtgagaatcttctcgacgaatcaaggaggattcatgttaccgatatacgagacagctgcaaaggcttacgctgtcggtcagttcgagcaaccgactgaaacgcctcctgaggacttagatacattgtctttggcgatagaagcagcgattcaggatcttagaaacaagagtcag

SEQ ID NO: 35 - DNA, 1488 bp, Artificial Sequence
  Synthetic polynucleotide IE2 VacO H363A/H369A
atgggagacatcctagcacaagcagtgaaccatgctggaattgactcatcttcgaccggaccaactctaacgactcattcatgttcggttagttctgctcctcttaacaagcctacacctacctcggtagctgttaccaacacacctttaccaggagcatcagcaacacctgagttgtctccaagaaagaagcctcgtaagaccacgagaccgttcaaggtgatcatcaagccaccagtaccacctgctccgatcatgttgccattgatcaagcaggaggacattaagccagaacctgacttcacgatacagtaccgtaacaagatcatagatacagcaggatgcatagtgatctcagatagtgaagaggagcaaggtgaggaagtggagactagaggagccacagccagttcgccttccacaggatccggaactcctagagtaactagtccgacacatccactttcccagatgaatcatccacctctaccggatcctctaggacgaccagatgaagattcttcttcatctagttcaagttcttgctcatccgcgagtgatagtgagtcagaaagtgaagagatgaagtgctcttctggtggtggagctagtgtcacttcatctcatcatggacgaggaggatttggaggtgctgcgagtagttccttactaagttgtggacatcagtcatctggtggtgcatctactggacctagaaagaagaagtcaaagagaatctccgaattggataatgagaaagtgagaaacatcatgaaggacaagaacacgccgttctgcactccgaatgttcagacgagaagaggacgagtgaagatagatgaagtatcacgaatgttcagaaacacaaatcgttctctagagtacaagaatcttccgttcaccataccttcgatgcaccaagtattagatgaggctatcaaggcatgtaagaccatgcaagttaacaacaaaggaatacagatcatctacactagaaaccatgaggttaagagtgaggtggatgccgtacgttgtagattgggaacgatgtgtaaccttgcgctatctactcctttcctaatggaggctactatgcctgtgactgctcctcctgaagtggctcaaagaacagctgatgcttgtaacgaaggtgtgaaagctgcttggtccctaaaggagttacatacacaccaactttgtccacgatccagtgactacagaaacatgatcattcatgcagctacgcctgtagatctacttggagctcttaacctatgtcttcctttgatgcagaagttccctaagcaagtgatggtgagaatcttctcgacgaatcaaggaggattcatgttaccgatatacgagacagctgcaaaggcttacgctgtcggtcagttcgagcaaccgactgaaacgcctcctgaggacttagatacattgtctttggcgatagaagcagcgattcaggatcttagaaacaagagtcag

SEQ ID NO: 36 - protein, 496 aa, Artificial Sequence
  Synthetic polypeptide IE2-VacO
MGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEHTMPVTHPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 37 - protein, 496 aa, Artificial Sequence; identical to SEQ ID NO: 36
  Synthetic polypeptide IE2

SEQ ID NO: 38 - protein, 496 aa, Artificial Sequence; identical to SEQ ID NO: 36
  Synthetic polypeptide IE2-4nt

SEQ ID NO: 39 - protein, 496 aa, Artificial Sequence
  Synthetic polypeptide IE2 H363A
MGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEATMPVTHPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 40 - protein, 496 aa, Artificial Sequence
  Synthetic polypeptide IE2 H369A
MGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEHTMPVTAPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 41 - protein, 496 aa, Artificial Sequence
  Synthetic polypeptide IE2 H363A/H369A
MGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEATMPVTAPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 42-43 - skipped (2 entries)

SEQ ID NO: 44 - DNA, 1224 bp, Artificial Sequence
  Synthetic polynucleotide IE1 VacO
atggtgaagcaaatcaaggtcagagtggacatggtaagacacagaattaaggaacacatgttgaagaagtatactcaaacagaggagaagttcaccggtgccttcaatatgatgggtggatgtctacagaacgctttggatatcttagataaggtacatgaaccattcgaagaaatgaagtgcattggattgacaatgcaatcaatgtatgagaactacatagtgccagaggataagcgtgaaatgtggatggcatgcatcaaggagttacatgatgtatccaaaggagcagccaacaagctaggtggtgctttgcaagcgaaggcaagagcgaagaaggatgaattgagacgaaagatgatgtacatgtgctatcgaaacatcgaattcttcactaagaactcagcgtttcctaagactaccaatggatgcagtcaagctatggctgcgcttcagaacttgcctcaatgtagtcctgatgaaatcatggcatatgcacagaagatcttcaagatcttagatgaggaaagagacaaggtattgactcatatcgatcacatattcatggatatactaacaacatgtgtagaaacgatgtgtaacgagtacaaggtaacttcggacgcttgtatgatgactatgtacggaggaatatctctacttagtgagttctgtcgagttctatgctgttacgtattagaagaaactagtgtaatgttagcgaagagaccattgatcactaagcctgaagtgatctcggttatgaagagacgaatagaggagatctgtatgaaggtgttcgcacaatacatcttaggagctgatcctctaagagtgtgtagtccatcggtagacgatttgagagctatagcggaggaatctgacgaggaagaggcaatagttgcatacacacttgctacagctggagtatccagttctgattctcttgtaagtcctccggagtcacctgtgccagcaaccataccgttgagtagtgtgattgtggctgagaactcggatcaggaagagtctgagcaatccgatgaagaagaggaggaaggagcacaagaggagagagaagatactgtctctgtgaagagtgaacctgtatctgaaatcgaggaagtagcacctgaggaagaggaggatggagccgaagaaccaacagcttcgggtggtaagtcaactcatccgatggtaaccagatctaaggcagaccagtaa

SEQ ID NO: 45 - DNA, 1194 bp, Artificial Sequence
  Synthetic polynucleotide IE1
atggtgcggcatagaatcaaggagcacatgctgaaaaaatatacccagacggaagagaaattcactggcgcctttaatatgatgggaggatgtttgcagaatgccttagatatcttagataaggttcatgagcctttcgaggagatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcactgcagaacttgcctcagtgctcccctgatgagattatggcttatgcccagaaaatatttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctgggggccgatcctctgagagtctgctctcctagtgtggatgacctacgggccatcgccgaggagtcagatgaggaagaggctattgtagcctacactttggccaccgctggtgtcagctcctctgattctctggtgtcacccccagagtcccctgtacccgcgactatccctctgtcctcagtaattgtggctgagaacagtgatcaggaagaaagtgagcagagtgatgaggaagaggaggagggtgctcaggaggagcgggaggacactgtgtctgtcaagtctgagccagtgtctgagatagaggaagttgccccagaggaagaggaggatggtgctgaggaacccaccgcctctggaggcaagagcacccaccctatggtgactagaagcaaggctgaccagtaa

SEQ ID NO: 46 - DNA, 1224 bp, Artificial Sequence
  Synthetic polynucleotide IE1-4nt
atggtcaaacagattaaggttcgagtggacatggtgcggcatagaatcaaggagcacatgctgaagaagtatacccagacggaagagaaattcactggcgcctttaatatgatgggaggatgtttgcagaatgccttagatatcttagataaggttcatgagcctttcgaggagatgaagtgtattgggctaactatgcagagcatgtatgagaactacattgtacctgaggataagcgggagatgtggatggcttgtattaaggagctgcatgatgtgagcaagggcgccgctaacaagttaggaggtgcactgcaggctaaggcccgtgctaagaaggatgaacttaggagaaagatgatgtatatgtgctacaggaatatagagttctttaccaagaactcagccttccctaagaccaccaatggctgcagtcaggccatggcggcactgcagaacttgcctcagtgctctcctgatgagattatggcttatgcccagaagatatttaagatcttggatgaggagagagacaaggtgctcacgcacattgatcacatatttatggatatcctcactacatgtgtggaaacaatgtgtaatgagtacaaggtcactagtgacgcttgtatgatgaccatgtacggaggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctaggtgccgatcctctgagagtctgctctcctagtgtggatgacctacgggccatcgccgaggagtcagatgaggaagaggctattgtagcctacactttggccaccgctggtgtcagctcctctgattctctggtgtcacctccagagtcacctgtacccgcgactatccctctgtcctcagtaattgtggctgagaacagtgatcaggaagaaagtgagcagagtgatgaggaagaggaggagggtgctcaggaggagcgggaggacactgtgtctgtcaagtctgagccagtgtctgagatagaggaagttgctccagaggaagaggaggatggtgctgaggaacccaccgcctctggaggcaagagcacccaccctatggtgactagaagcaaggctgaccagtaa

(19) United States
(12) Patent Application Publication
(10) Pub. No.: US 2023/0381299 A1
(43) Pub. Date: Nov. 30, 2023

(54) VACCINATION OF HEMATOPOIETIC STEM CELL DONORS WITH CYTOMEGALOVIRUS TRIPLEX COMPOSITION

(71) Applicant: CITY OF HOPE, Duarte, CA (US)

(72) Inventors: Don J. DIAMOND, Duarte, CA (US); Corinna LA ROSA, Duarte, CA (US)

(73) Assignee: CITY OF HOPE, Duarte, CA (US)

(21) Appl. No.: 18/032,052

(22) PCT Filed: Oct. 15, 2021
(86) PCT No.: PCT/US21/55220

§ 371 Date: Apr. 14, 2023

Related U.S. Application Data
(60) Provisional application No. 63/092,741, filed on Oct. 16, 2020.

(51) Int. Cl. A61K 39/245; A61K 39/295; C12N 15/86

(52) U.S. Cl. CPC A61K 39/245; A61K 39/295; C12N 15/86; A61K 2039/5256

(57) ABSTRACT

Disclosed is a method of treating or preventing a CMV infection in a recipient of a hematopoietic cell transplant (HCT). The method entails administering an effective amount of a CMV Triplex vaccine composition to a donor and/or recipient of the hematopoietic cells.

### PRIORITY CLAIM

This application claims priority to U.S. Provisional Application No. 62/670,656, filed on May 11, 2018, which is incorporated by reference herein in its entirety, including drawings.

### STATEMENT OF GOVERNMENT INTEREST

The present invention was made with government support under Grant No. CA077544 awarded by the National Cancer Institute of the National Institutes of Health (NIH). The government has certain rights in the invention.

### BACKGROUND

Modified Vaccinia Ankara (MVA) is a genetically engineered, highly attenuated strain of vaccinia virus that does not propagate in most mammalian cells. This property minimally impacts viral or foreign gene expression because the ability of MVA to propagate in mammalian cells is blocked at late stage viral assembly. However, the DNA continues to replicate and therefore acts as an efficient template for RNA biosynthesis leading to high levels of protein synthesis. MVA also has a large foreign gene capacity and multiple integration sites, two features that make it a desirable vector for expressing vaccine antigens. MVA has a well-established safety record and versatility for the production of heterologous proteins. In fact, MVA-based vaccines for treatment of infectious disease and cancer have been developed and reached Phase I/II clinical trials.

MVA has an extensive history of successful delivery into rodents, Rhesus macaques, and other non-human primates, and more recently as a clinical vaccine in cancer patients. The original MVA virus was administered to 120,000 young and elderly in Europe in the 1970s. MVA is avirulent because of the loss of two important host-range genes among 25 mutations and deletions that occurred during its repeated serial passage in chicken cells.

MVA is appealing as a vaccine vector for CMV antigens in individuals who are both severely immunosuppressed and experiencing additional complications such as malignancy or organ failure, thereby requiring a transplant. CMV infection is an important complication of transplantation procedures and affects a wide variety of individuals including newborns and HIV patients with advanced disease. Human cytomegalovirus (HCMV) is a major risk factor for recipients of solid organ and hematopoietic stem cell transplants. Individuals who are previously CMV-infected or receiving a CMV-infected solid organ or stem cell allograft are candidates for a vaccine strategy that targets the cellular reservoir of the virus.

It has been reported that in vitro expression levels of foreign antigens by an rMVA vaccine are correlated with the rMVA vaccine's immunogenicity. However, after serial passage, the foreign antigen expression may be reduced, which can result in diminished immunogenicity. Thus, while MVA is an attractive viral vector for recombinant vaccine development, genetic instability and diminished immunogenicity are significant concerns after serial passage. The beneficial effect of high antigen expression levels and improved immunogenicity can be limited by the propensity of rMVA to delete genes unnecessary for its lifecycle.

A first generation “Triplex” vaccine was constructed to attenuate or suppress ongoing CMV viremia and its propagation. The first-generation Triplex includes three immunodominant proteins: pp65 (major tegument protein) and a fusion between immediate early proteins IE1 and IE2 (IEfusion). These antigens have previously been combined and expressed in a single MVA vector; however, the current assembly of these antigens within MVA is not optimal for mass production of a vaccine. Upon extended viral passage, a decrease in expression of IEfusion was observed. This vaccine was successfully evaluated in a Phase I safety and dose escalation trial in 24 healthy volunteers [31].

It will be advantageous to develop an rMVA vaccine with improved stable expression of foreign protein antigens and potent immunogenicity after extended serial passage, which will enable large scale manufacturing of MVA expressing certain HCMV antigens as a clinical vector for a broader portfolio of infectious pathogens and cancer antigens.

### SUMMARY

In one aspect, this disclosure is directed to an expression system for co-expressing two or more cytomegalovirus (CMV) antigens, e.g. human CMV antigens. The expression system includes a genetically recombinant modified Vaccinia Ankara (rMVA) vector inserted with two or more nucleic acid sequences encoding two or more CMV antigens or antigenic portions thereof. In some embodiments, the CMV antigens or antigenic fragments thereof include IE1 exon 4 (IE1/e4), IE2 exon 5 (IE2/e5), IEfusion (e.g. fusion of IE1/e4 and IE2/e5), and pp65. In various embodiments, pp65 can be co-expressed with IE1/e4, IE2/e5, or IEfusion. The expression system can co-express the CMV antigens simultaneously from a single vector. In some embodiments, the nucleic acid sequences encoding the two or more CMV antigens are inserted in one or more insertion sites including 044L/045L, IGR3, G1L/I8R, and Del3. Additional insertion sites include those listed in Table 1.

In some embodiments, two or more nucleic acid sequences are operably linked to and under the control of a single promoter, such as the mH5 promoter. In other embodiments, each nucleic acid sequence is operably linked to and under the control of a separate mH5 promoter. Additionally, other poxvirus promoters can be used and the use of an mH5 promoter is not required. In some embodiments, one or more nucleic acid sequences are codon optimized to remove consecutive cytosines or guanines while expressing without alteration of the same amino acids. In some embodiments, the amino acid sequences of the CMV antigens comprise one or more mutations to improve the genetic stability of the rMVA upon viral passaging. In some embodiments, IE1 and IE2 or antigenic fragments thereof are expressed as an IE fusion protein such as a fusion of IE1/exon 4 and IE2/exon 5. In some embodiments, the rMVA expressing the CMV antigens is genetically stable for at least 10 passages.

Another aspect of this disclosure is directed to a vaccine comprising an immunologically effective amount of the recombinant modified vaccinia Ankara (rMVA) disclosed herein which is genetically stable after at least 10 passages.

Another aspect of this disclosure is directed to a method of eliciting or modifying an immune response and clinical protection against viremia and diseases caused by uncontrolled propagation of CMV in a subject by administering a vaccine composition as described above to the subject. In some embodiments, the subject is a mammal, such as a human.

Yet another aspect of this disclosure is directed to a method of improving the stability upon passage of an rMVA expressing two or more CMV antigens or antigenic fragments thereof by incorporating one or more of the following modifications: (1) inserting one or more nucleic acid sequences encoding the CMV antigens or antigenic fragments thereof into one or more insertion sites including 044L/045L, IGR3, G1L/I8R, and Del3, as well as additional insertion sites listed in Table 1, not including Del2; (2) codon optimizing the nucleic acid sequences encoding the CMV antigens by removing consecutive cytosines or guanines; and (3) introducing one or more mutations in the amino acid sequences of the CMV antigens. In some embodiments, the CMV antigens or antigenic fragments thereof include IE1 exon 4 (IE1/e4), IE2 exon 5 (IE2/e5), IEfusion (e.g. fusion of IE1 and IE2 or IE1/e4 and IE2/e5), and pp65.

### BRIEF DESCRIPTION OF THE DRAWINGS

FIG. 1 shows a schematic diagram portraying the development of the rederived Triplex. MVA BAC expressing pp65 is used as the basis for the addition of other HCMV antigens for Triplex. Utilizing BAC technology and en passant mutagenesis, genes expressing desired Triplex antigens are sequentially incorporated into MVA. After all desired antigens are present and the final constructs have been analyzed for stability, BAC is removed from MVA. Solid black arrows signify final rederived Triplex construct; gray arrows signify intermediate steps. There are three examples of the final potential Triplex candidates: I) IE2, IE1, and pp65 in different sites on MVA; II) IEfusion in 044L/045L; and III) IEfusion in IGR3. All three example constructs have pp65 in Del3; (*) signifies variants of genes that are inserted into the sites.

FIGS. 2A-2C show the Triplex gene organization in Modified Vaccinia Ankara (MVA) virus and its stability post-passaging in chicken embryonic fibroblasts (CEFs). FIG. 2A shows an abbreviated schematic of the original construction of IEfusion in Triplex. HCMV AD169 exons 4 and 5 for IE1 and IE2, respectively, were engineered with an Apal site for a seamless junction, resulting in IEfusion. IEfusion was inserted into the Del2 site of MVA, controlled by the mH5 promoter. In the MVA Del3 site is pp65, also controlled by the mH5 promoter. FIG. 2B shows Western blot analysis of clinical Triplex passaged up to seven additional times (P6-P12) in CEF. Lane labeled “CEF” is uninfected, negative control; lane labeled “MVA” is wild-type MVA not expressing CMV antigens; lane labeled “Triplex” is virus used to generate clinical lots of Triplex at P5. IEfusion was probed using a purified anti-IE1 mouse monoclonal antibody (mAB) p63-27 [11]; pp65 was probed using purified mouse mAB 28-103 [4]. As an infection/loading control, BR5 antibody was used to probe against an envelope MVA glycoprotein. FIG. 2C shows 1% agarose gel visualizing PCR-amplification of IEfusion in Del2 from P6-P12, with primers flanking the gene within the Del2 site.

FIGS. 3A-3D show the nucleic acid sequence alignment of IEfusion constructs with mutations to the consecutive cytosines and guanines thereby reducing instability and/or vaccinia codon optimization for protein expression. SEQ ID NOs: 1-3 and 25.

FIGS. 4A-4C show the stability analysis of IEfusion 4nt in IGR3 insertion site on MVA-BAC. FIG. 4A shows a schematic representing the insertion of IEfusion (4nt) into the IGR3 site (shown with an arrow). All evaluated MVA BAC insertion sites, G1L/I8R, 044L/045L, and IGR3, are noted with arrows. FIG. 4B shows 1% agarose gel of PCR product analyzing stability of IEfusion (4nt) in IGR3, passaged up to ten times (P1-P10) in chicken embryonic fibroblasts (CEFs). FIG. 4C shows Western blot analysis of IEfusion (4nt) passaged up to P10 in CEF cells. IEfusion was probed using a purified anti-IE1 mouse monoclonal antibody (mAB) p63-27 [11]; pp65 was probed using purified mouse mAB 28-103 [4]. As an infection/loading control, BR5 antibody was used to probe against an envelope MVA glycoprotein. For FIGS. 4B and 4C, lane labeled “CEF” is uninfected, negative control; lane labeled “MVA” is wildtype MVA not expressing any antigen; lane labeled “Triplex” is virus (at P6) used to generate clinical lots of Triplex.

FIG. 5 is a schematic of potential sites on MVA-BAC and de-construction of IEfusion into IE1 and IE2. The potential sites in MVA BAC available for insertion of either IE1 or IE2 genes are 044L/045L, IGR3, and G1L/I8R. Upon separation of IE1 and IE2, each gene is to be controlled by the mH5 promoter.

FIGS. 6A-6E show the nucleic acid sequence alignment of constructs expressing IE2 protein variants (6A-6C) (SEQ ID NOs: 7-9 and 26) and the nucleic acid sequence alignment of constructs expressing IE1 protein variants (6D-6E). SEQ ID NOs: 27-30.

FIGS. 7A-7C show the stability analysis of IE2 in 044L/045L insertion site on MVA. FIG. 7A shows a schematic representing the insertion of IE2 into the 044L/045L site (shown with an arrow). FIG. 7B shows 1% agarose gel of PCR product analyzing stability of IE2 in 044L/045L, passaged up to ten times (P1-P10) in chicken embryonic fibroblasts (CEFs). FIG. 7C shows Western blot analysis of IE2 passaged up to P10 in CEF cells. IE2 was probed using an anti-IE2 mouse monoclonal antibody (mAB) 2.9.5 [11]; pp65 was probed using purified mouse mAB 28-103 [4]. As an infection/loading control, BR5 antibody was used to probe against an envelope MVA glycoprotein. For FIGS. 7B and 7C, lane labeled “CEF” is uninfected, negative control; lane labeled “MVA” is wildtype MVA not expressing any antigen; lane labeled “Triplex” is virus used to generate clinical lots of Triplex at P6.

FIGS. 8A-8C show the stability analysis of IE2 H363A mutant in 044L/045L insertion site on MVA. FIG. 8A shows a schematic representation of IE2, showing the location of two histidine residues at positions 363 and 369 (H363 and H369) within the specific and essential modulator (SEM, heavily shaded gray with corresponding arrow) and core (lightly shaded with corresponding arrow). Within the IE2 amino acid sequence, an internal TATA box for the transcription of a putative ˜40 kDa product is labeled [18]. This amino acid annotation is consistent with the amino acid numbers corresponding to IE2 lacking a nuclear localization signal or a signal peptide. FIG. 8B shows 1% agarose gel of PCR product analyzing stability of IE2 H363A in 044L/045L, passaged up to ten times (P1-P10) in chicken embryonic fibroblasts (CEFs). FIG. 8C shows Western blot analysis of IE2 H363A passaged up to P10 in CEF cells. IE2 H363A was probed using an anti-IE2 mouse monoclonal antibody (mAB) 2.9.5 [11]. As an infection/loading control, BR5 antibody was used to probe against an envelope MVA glycoprotein. For FIGS. 8B and 8C, lane labeled “CEF” is uninfected, negative control; lane labeled “MVA” is wild-type MVA not expressing any antigen; lane labeled “IE2 (044L/045L)” is virus previously shown to express non-codon optimized IE2 (FIG. 7C).

FIGS. 9A-9C show the stability analysis of IE1 NCO, 4nt, and VacO in G1L/I8R insertion site on MVA-BAC. PCR (left) and Western blot analyses (right) of IE1 NCO (9A), 4nt (9B), and VacO (9C). Left: 1.0% agarose gel of PCR product analyzing stability of IE1 in G1L/I8R, passaged up to P10 in CEF. Right: Western blot analysis of IE1 passaged up to P10 in CEF cells. IE1 and IEfusion was probed using a purified anti-IE1 mouse monoclonal antibody (mAB) p63-27. As an infection/loading control (bottom), BR5 antibody was used to probe against an envelope MVA glycoprotein. “CEF” is uninfected, negative control; lane labeled “MVA” is wild-type MVA not expressing any antigen; lane labeled (+) is virus used to generate clinical lots of Triplex.

FIGS. 10A-106 show the stability analysis of IE1 4nt and VacO in IGR3 insertion site on MVA-BAC. PCR (left) and Western blot analyses (right) of IE1 4nt (10A), and VacO (10B). Left: 1.0% agarose gel of PCR product analyzing stability of IE1 IGR3, passaged up to P10 in CEF. Right: Western blot analysis of IE1 passaged up to P10 in CEF cells. IE1 and IEfusion were probed using a purified anti-IE1 mouse monoclonal antibody (mAB) p63-27. As an infection/loading control (bottom), BR5 antibody was used to probe against an envelope MVA glycoprotein. “CEF” is uninfected, negative control; lane labeled “MVA” is wild-type MVA not expressing any antigen; lane labeled (+) is virus used to generate clinical lots of Triplex.

FIGS. 11A-116 show stability analysis of IE2 NCO and 4nt in G1L/I8R insertion site on MVA-BAC. PCR (left) and Western blot analyses (right) of IE2 NCO (11A) and 4nt (11B). Left: 1.0% agarose gel of PCR product analyzing stability of IE2 G1L/I8R, passaged up to P10 in CEF. Right: Western blot analysis of IE2 passaged up ten times (P1-P10) in CEF cells. IE2 was probed using an anti-IE2 mAB 2.9.5. As an infection/loading control (bottom), BR5 antibody was used to probe against an envelope MVA glycoprotein. “CEF” is uninfected, negative control; lane labeled “MVA” is wild-type MVA not expressing any antigen; lane labeled (+) is virus used to generate clinical lots of Triplex.

FIG. 12 shows stability analysis of IE2 VacO in IGR3 insertion site on MVA-BAC. PCR (left) and Western blot analyses (right) of IE2 VacO. Left: 1.0% agarose gel of PCR product analyzing stability of IE2 IGR3, passaged up to P10 in CEF. Right: Western blot analysis of IE2 passaged up to P10 in CEF cells. IE2 was probed using an anti-IE2 mAB 2.9.5. As an infection/loading control (bottom), BR5 antibody was used to probe against an envelope MVA glycoprotein. “CEF” is uninfected, negative control; lane labeled “MVA” is wild-type MVA not expressing any antigen; lane labeled (+) is virus used to generate clinical lots of Triplex.

FIGS. 13A-13B show stability analysis of IE2H363A NCO and 4nt in 044/045L insertion site on MVA-BAC. PCR (left) and Western blot analyses (right) of IE2 NCO (13A) and 4nt (13B). Left: 1.0% agarose gel of PCR product analyzing stability of IE2 mutants in 044/045L, passaged up to P10 in CEF. Right: Western blot analysis of IE2 mutants passaged up to P10 in CEF cells. IE2 was probed using an anti-IE2 mAB 2.9.5. As an infection/loading control (bottom), BR5 antibody was used to probe against an envelope MVA glycoprotein. “CEF” is uninfected, negative control; lane labeled “MVA” is wild-type MVA not expressing any antigen; lane labeled (+) is virus used to generate clinical lots of Triplex.

FIGS. 14A-14C show stability analysis of IE2H369A NCO, 4nt, and VacO in 044/045L insertion site on MVA-BAC. PCR (left) and Western blot analyses (right) of IE2 NCO (14A), 4nt (14B), and VacO (14C). Left: 1.0% agarose gel of PCR product analyzing stability of IE2 mutants in 044/045L, passaged up to P10 in CEF. Right: Western blot analysis of IE2 mutants passaged up to P10 in CEF cells. IE2 was probed using an anti-IE2 mAB 2.9.5. As an infection/loading control (bottom), BR5 antibody was used to probe against an envelope MVA glycoprotein. “CEF” is uninfected, negative control; lane labeled “MVA” is wild-type MVA not expressing any antigen; lane labeled (+) is virus used to generate clinical lots of Triplex.

FIGS. 15A-15C show stability analysis of IE2H363/369A NCO, 4nt, and VacO in 044/045L insertion site on MVA-BAC. PCR (left) and Western blot analyses (right) of IE2 NCO (15A), 4nt (15B), and VacO (15C). Left: 1.0% agarose gel of PCR product analyzing stability of IE2 mutants in 044/045L, passaged up to P10 in CEF. Right: Western blot analysis of IE2 mutants passaged up to P10 in CEF cells. IE2 was probed using an anti-IE2 mAB 2.9.5. As an infection/loading control (bottom), BR5 antibody was used to probe against an envelope MVA glycoprotein. “CEF” is uninfected, negative control; lane labeled “MVA” is wild-type MVA not expressing any antigen; lane labeled (+) is virus used to generate clinical lots of Triplex.

FIGS. 16A-16C show stability analysis of IEfusion 4nt mutants in IGR3 insertion site on MVA-BAC. PCR (left) and Western blot analyses (right) of IEfusion 4nt H363A (16A), H369A (16B), and H363A/H369A (16C). Left: 1.0% agarose gel of PCR product analyzing stability of IEfusion mutants in IGR3, passaged up to P10 in CEF. Right: Western blot analysis of IEfusion mutants passaged up to P10 in CEF cells. IEfusion was probed using a purified anti-IE1 mouse monoclonal antibody (mAB) p63-27. As an infection/loading control (bottom), BR5 antibody was used to probe against an envelope MVA glycoprotein. “CEF” is uninfected, negative control; lane labeled “MVA” is wild-type MVA not expressing any antigen; lane labeled (+) is virus used to generate clinical lots of Triplex.

FIG. 17 shows stability analysis of construct A(i). PCR (left) and Western blot analyses (right) of IE1, IE2, and pp65. Left: 1.0% agarose gel of PCR product analyzing stability of IE1 in IGR3, IE2 in 044/045L, and pp65 in Del3, passaged up to P10 in CEF. Right: Western blot analysis of IEfusion and pp65 passaged up to P10 in CEF cells. IE1 was probed using a purified anti-IE1 mouse monoclonal antibody (mAB) p63-27; IE2 was probed using an anti-IE2 mAB 2.9.5; and pp65 was probed using purified mouse mAB 28-103. As an infection/loading control (bottom), BR5 antibody was used to probe against an envelope MVA glycoprotein. “CEF” is uninfected, negative control; lane labeled “MVA” is wild-type MVA not expressing any antigen; lane labeled (+) is virus used to generate clinical lots of Triplex.

FIG. 18 shows stability analysis of construct A(v). PCR (left) and Western blot analyses (right) of IE1, IE2, and pp65. Left: 1.0% agarose gel of PCR product analyzing stability of IE1 in IGR3, IE2 in 044/045L, and pp65 in DeI3, passaged up to P10 in CEF. Right: Western blot analysis of IEfusion and pp65 passaged up to P10 in CEF cells. IE1 was probed using a purified anti-IE1 mouse monoclonal antibody (mAB) p63-2; IE2 was probed using an anti-IE2 mAB 2.9.5; and pp65 was probed using purified mouse mAB 28-103. As an infection/loading control (bottom), BR5 antibody was used to probe against an envelope MVA glycoprotein. “CEF” is uninfected, negative control; lane labeled “MVA” is wild-type MVA not expressing any antigen; lane labeled (+) is virus used to generate clinical lots of Triplex.

FIG. 19 shows stability analysis of construct A(vi). PCR (left) and Western blot analyses (right) of IE1, IE2, and pp65. Left: 1.0% agarose gel of PCR product analyzing stability of IE1 in IGR3, IE2 in 044/045L, and pp65 in DeI3, passaged up to P10 in CEF. Right: Western blot analysis of IEfusion and pp65 passaged up to P10 in CEF cells. IE1 was probed using a purified anti-IE1 mouse monoclonal antibody (mAB) p63-27; IE2 was probed using an anti-IE2 mAB 2.9.5; and pp65 was probed using purified mouse mAB 28-103. As an infection/loading control (bottom), BR5 antibody was used to probe against an envelope MVA glycoprotein. “CEF” is uninfected, negative control; lane labeled “MVA” is wild-type MVA not expressing any antigen; lane labeled (+) is virus used to generate clinical lots of Triplex.

FIG. 20 shows stability analysis of construct B(i). PCR (left) and Western blot analyses (right) of IE1, IE2, and pp65. Left: 1.0% agarose gel of PCR product analyzing stability of IE1 in IGR3, IE2 in 044/045L, and pp65 in DeI3, passaged up to P10 in CEF. Right: Western blot analysis of IEfusion and pp65 passaged up to P10 in CEF cells. IE1 was probed using a purified anti-IE1 mouse monoclonal antibody (mAB) p63-27; IE2 was probed using an anti-IE2 mAB 2.9.5; and pp65 was probed using purified mouse mAB 28-103. In this figure (+) did not work, hence no bands observed for α-pp65 or α-IE1 Western blot. As an infection/loading control (bottom), BR5 antibody was used to probe against an envelope MVA glycoprotein. “CEF” is uninfected, negative control; lane labeled “MVA” is wild-type MVA not expressing any antigen; lane labeled (+) is virus used to generate clinical lots of Triplex.

FIG. 21 shows stability analysis of construct B(ii). PCR (left) and Western blot analyses (right) of IE1, IE2, and pp65. Left: 1.0% agarose gel of PCR product analyzing stability of IE1 in IGR3, IE2 in 044/045L, and pp65 in DeI3, passaged up to P10 in CEF. Right: Western blot analysis of IEfusion and pp65 passaged up to P10 in CEF cells. IE1 was probed using a purified anti-IE1 mouse monoclonal antibody (mAB) p63-27; IE2 was probed using an anti-IE2 mAB 2.9.5; and pp65 was probed using purified mouse mAB 28-103. As an infection/loading control (bottom), BR5 antibody was used to probe against an envelope MVA glycoprotein. “CEF” is uninfected, negative control; lane labeled “MVA” is wild-type MVA not expressing any antigen; lane labeled (+) is virus used to generate clinical lots of Triplex. (*) for P1 PCR indicates missing lane.

FIG. 22 shows stability analysis of construct B(iii). PCR (left) and Western blot analyses (right) of IE1, IE2, and pp65. Left: 1.0% agarose gel of PCR product analyzing stability of IE1 in IGR3, IE2 in 044/045L, and pp65 in DeI3, passaged up to P10 in CEF. Right: Western blot analysis of IEfusion and pp65 passaged up to P10 in CEF cells. IE1 was probed using a purified anti-IE1 mouse monoclonal antibody (mAB) p63-27; IE2 was probed using an anti-IE2 mAB 2.9.5; and pp65 was probed using purified mouse mAB 28-103. As an infection/loading control (bottom), BR5 antibody was used to probe against an envelope MVA glycoprotein. “CEF” is uninfected, negative control; lane labeled “MVA” is wild-type MVA not expressing any antigen; lane labeled (+) is virus used to generate clinical lots of Triplex. For clarity of protein identification based on molecular weight, IEfusion and IE1 are indicated by arrows.

FIG. 23 shows stability analysis of construct B(v). PCR (left) and Western blot analyses (right) of IE1, IE2, and pp65. Left: 1.0% agarose gel of PCR product analyzing stability of IE1 in IGR3, IE2 in 044/045L, and pp65 in DeI3, passaged up to P10 in CEF. Right: Western blot analysis of IEfusion and pp65 passaged up to P10 in CEF cells. IE1 was probed using a purified anti-IE1 mouse monoclonal antibody (mAB) p63-27; IE2 was probed using an anti-IE2 mAB 2.9.5; and pp65 was probed using purified mouse mAB 28-103. As an infection/loading control (bottom), BR5 antibody was used to probe against an envelope MVA glycoprotein. “CEF” is uninfected, negative control; lane labeled “MVA” is wild-type MVA not expressing any antigen; lane labeled (+) is virus used to generate clinical lots of Triplex.

FIG. 24 shows stability analysis of construct B(vii). PCR (left) and Western blot analyses (right) of IE1, IE2, and pp65. Left: 1.0% agarose gel of PCR product analyzing stability of IE1 in IGR3, IE2 in 044/045L, and pp65 in DeI3, passaged up to P10 in CEF. Right: Western blot analysis of IEfusion and pp65 passaged up to P10 in CEF cells. IE1 was probed using a purified anti-IE1 mouse monoclonal antibody (mAB) p63-27; IE2 was probed using an anti-IE2 mAB 2.9.5; and pp65 was probed using purified mouse mAB 28-103. As an infection/loading control (bottom), BR5 antibody was used to probe against an envelope MVA glycoprotein. “CEF” is uninfected, negative control; lane labeled “MVA” is wild-type MVA not expressing any antigen; lane labeled (+) is virus used to generate clinical lots of Triplex.

FIG. 25 shows stability analysis of IEfusion 4nt H363A (IGR3):pp65(Del3). PCR (left) and Western blot analyses (right) of IEfusion 4nt H363A and pp65. Left: 1.0% agarose gel of PCR product analyzing stability of IEfusion in IGR3 and pp65 in DeI3, passaged up to P10 in CEF. Right: Western blot analysis of IEfusion and pp65 passaged up to P10 in CEF cells. IEfusion was probed using a purified anti-IE1 mouse monoclonal antibody (mAB) p63-27; pp65 was probed using purified mouse mAB 28-103. As an infection/loading control (bottom), BR5 antibody was used to probe against an envelope MVA glycoprotein. “CEF” is uninfected, negative control; lane labeled “MVA” is wild-type MVA not expressing any antigen; lane labeled (+) is virus used to generate clinical lots of Triplex.

FIG. 26 shows stability analysis of IEfusion 4nt H369A (IGR3):pp65(Del3). PCR (left) and Western blot analyses (right) of IEfusion 4nt H369A and pp65. Left: 1.0% agarose gel of PCR product analyzing stability of IEfusion in IGR3 and pp65 in DeI3, passaged up to P10 in CEF. Right: Western blot analysis of IEfusion and pp65 passaged up to P10 in CEF cells. IEfusion was probed using a purified anti-IE1 mouse monoclonal antibody (mAB) p63-27; pp65 was probed using purified mouse mAB 28-103. As an infection/loading control (bottom), BR5 antibody was used to probe against an envelope MVA glycoprotein. “CEF” is uninfected, negative control; lane labeled “MVA” is wild-type MVA not expressing any antigen; lane labeled (+) is virus used to generate clinical lots of Triplex.

FIGS. 27A-27B show T-cell responses and stimulation post second-generation Triplex immunization. FIG. 27A: Human MHC-restricted T-cell responses elicited by second-generation Triplex. Graphical representation of data from Table 6. FIG. 27B: HLA-B*0702- or HLA-A*0201-restricted CD8+ T-cell stimulation by second-generation Triplex. Graphical representation of data from Table 7. Error bars are SEM calculated and reported in Tables 6 and 7.

FIGS. 28A-28B show stability analysis of constructs expressing duplicate IE2 genes. PCR (top) and Western blot analyses (bottom) of IE2 and IE2 mutants. FIG. 28A: 1.0% agarose gel of PCR product analyzing stability of IE2 in G1L and three versions of IE2 mutants in 044/045L, passaged up to P5 in CEF. FIG. 28B: Western blot analysis of IE2 and three mutants were passaged up P5 in CEF cells. IE2 was probed using an anti-IE2 mAB 2.9.5. As an infection/loading control (bottom), BR5 antibody was used to probe against an envelope MVA glycoprotein. “CEF” is uninfected, negative control; lane labeled “MVA” is wild-type MVA not expressing any antigen; lane labeled (+) is virus used to generate clinical lots of Triplex.

### DETAILED DESCRIPTION OF THE INVENTION

The current Triplex vaccine formulation includes three immunodominant proteins: pp65 and a fusion of immediate early proteins IE1 and IE2, but has restrictive manufacturing properties: 1) it must undergo limited passaging to maintain the stability of the IEfusion insertion; 2) restricted growth conditions to allow virus propagation without IEfusion instability; and 3) for mass production of large scale clinical lots, the current Triplex formulation is not the most efficient, long-term production strategy.

Utilizing the modified vaccinia Ankara (MVA) vaccine platform in combination with the bacterial artificial chromosome (BAC) technology, a new form of Triplex that stably expresses both IE1 and IE2 proteins in separate insertion sites over ten passages is generated. MVA is a well-characterized and clinically well-tolerated vaccine vector that is widely used for developing therapeutic vaccine strategies to treat or prevent infectious diseases or cancer. Induction of cellular immune responses by HCMV antigens IE1, IE2, and pp65 is thought to be imperative for the construction of a vaccine candidate to prevent infection or re-infection of individuals that have or will undergo hematopoietic stem cell or solid organ transplants. Disclosed herein are the construction of MVA vectors simultaneously expressing multiple HCMV antigens with insertion sites within MVA, modifications to the IE1 and IE2 components of IEfusion, and splitting IEfusion into its individual components of IE1 (exon 4) and IE2 (exon 5). The inserted HCMV antigen sequences are based on their natural HCMV DNA sequences or have been codon-optimized for efficient vaccinia virus expression. The individual HCMV antigens are separately inserted into three unique MVA insertion sites. There are four candidate insertion sites that include MVA deletion site III (Del3), a site between MVA essential genes I8R and G1 L, intergenic region IGR3, and MVA 044/045L site. An ectopically inserted modified promoter H5 induces expression of the HCMV antigens from the MVA vector. Furthermore, a His to Ala amino acid substitution on the C-terminal DNA-binding domain of IE2 has aided in the stable expression of IE2 over a minimum of ten passages. Therefore, His to Ala substitutions were inserted via site-directed mutagenesis to further stabilize IE2. These mutations have helped stabilize expression of IE2 through ten passages.

In one aspect, this disclosure relates to improving the stability upon extended passage of Triplex and to retaining immunogenicity while maintaining all three antigens needed for an effective vaccine formulation. For example, one or more modifications can be made to yield an MVA that stably expresses IE1, IE2, and pp65 for efficient viral vaccine production, including but not limited to: 1) use of multiple, unique gene insertion sites in MVA that could be the preferred environment for gene stability; 2) removal of DNA mutation “hot spots” within the gene sequence that have been previously identified to include mutations at the codon “wobble” position thereby disrupting consecutive C or G nucleotides; and 3) pox virus codon-optimization for increased protein expression. In some embodiments, IEfusion is inserted into other sites within MVA. Candidate sites include Del3 [5, 6], G1L/I8R [7, 8], IGR3 [9], and 044L/045L [10]. Additional insertion sites are listed in Table 1. In some embodiments, the insertion sites do not include Del2. In some embodiments, 3 or more, 4 or more, 5 or more, 6 or more consecutive C or G nucleotides in the gene sequence are disrupted by wobble base substitution that maintain identical amino acid identity.

Disclosed herein are the most stable combinations of insertion sites and gene modifications to generate an MVA that stably expresses all three CMV antigens at a minimum of 10 passages for large-scale propagation of the vaccine. Various combinations are contemplated to find the most stable combination of insertion sites that allows stable expression of IE1, IE2, and pp65: 1) splitting IEfusion into its IE1 and IE2 gene components; 2) inserting all three genes into separate insertion sites in MVA and using variant gene sequences of the inserts; and 3) explore new insertion sites in MVA. Some examples of the insertion sites are provided in Table 1:

TABLE 1
MVA Insertion Sites

| MVA Insertion Sites | Descriptive Name |
|---|---|
| IGR007/008 | |
| IGR021/023 | Del2 |
| IGR044/045 | 44L/45L |
| IGR047/048 | |
| IGR055/056 | |
| IGR064/065 | IGR3 |
| IGR069/070 | G1L/I8R |
| IGR081/082 | |
| IGR090/091 | |
| IGR092/093 | |
| IGR107/108 | |
| IGR116/117 | |
| IGR122/123 | |
| IGR136/137 | Del6 |
| IGR148/149 | |
| IGR164/165 | Del3 |

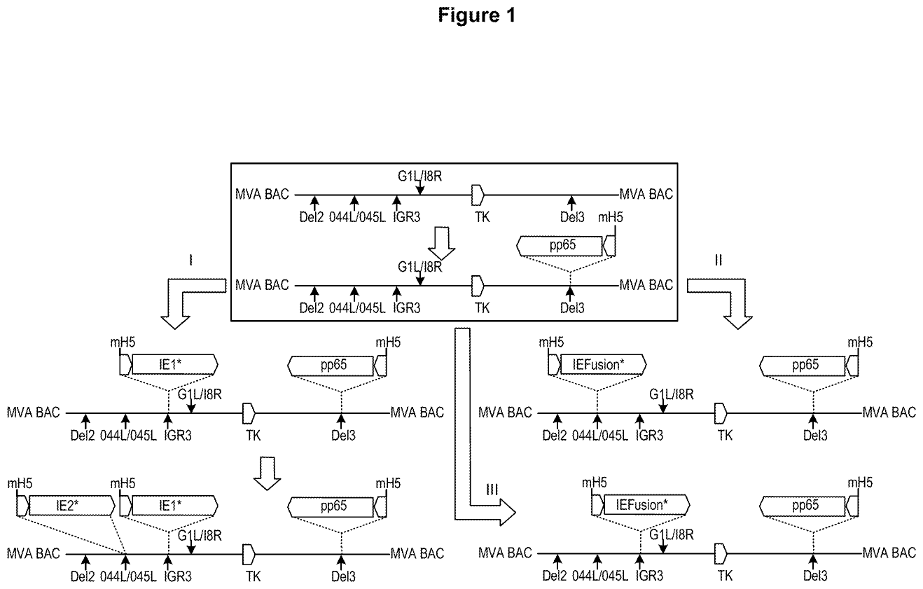

Various modifications and/or insertion sites selection are made with the purpose of increasing the stability of Triplex simultaneously expressing IE1, IE2 and pp65 in a single MVA vector, as illustrated in FIG. 1. Some MVA insertion sites provide for an environment that creates greater stability for either IE1 or IE2, possibly based on their nucleotide sequence. For example, one or more of the IE1, IE2 and pp65 genes are inserted in one or more insertion sites, including 044L/045L, IGR3, G1L/I8R, and Del3. In some embodiments, the insertion site does not include Del2. In addition or in the alternative, the DNA sequence of the IE1 gene and/or IE2 gene, and/or the amino acid sequence of the IE1 protein and/or IE2 protein is modified to be more compatible with the MVA life cycle or the absence of cell toxicity. In some embodiments, the DNA sequence of the IE1 gene and/or IE2 gene is codon optimized. For example, consecutive DNA sequences of cytosines or guanines are codon optimized and replaced by DNA sequences encoding the same amino acid residues without the consecutive cytosines or guanines. In some embodiments, the amino acid sequence of the IE1 protein or the IE2 protein contains one or more mutations such that the stability of the mutant IE1 protein or mutant IE2 protein is improved compared to that of the wildtype IE1 protein or wildtype IE2 protein. In some embodiments, one or more amino acid mutations disrupt the Zn-finger domain of the IE2 protein. For example, the amino acid sequence of the IE2 protein contains one or more His→Ala mutations in the C-terminus. In some embodiments, the amino acid sequence of the IE2 protein contains an H363A mutation, an H369A mutation, or both.

An “immunologically effective amount” as used herein means an amount that is both safe to a subject (animal or human) to be immunized and sufficient to improve the immunity of the subject. The immunologically effective amount can vary and can be determined by means of known art through routine trials.

In another embodiment, a CMV vaccine containing an immunologically effective amount of rMVA virus, which is genetically stable after serial passage can be produced by the methods disclosed herein, incorporating one or more modifications described above.

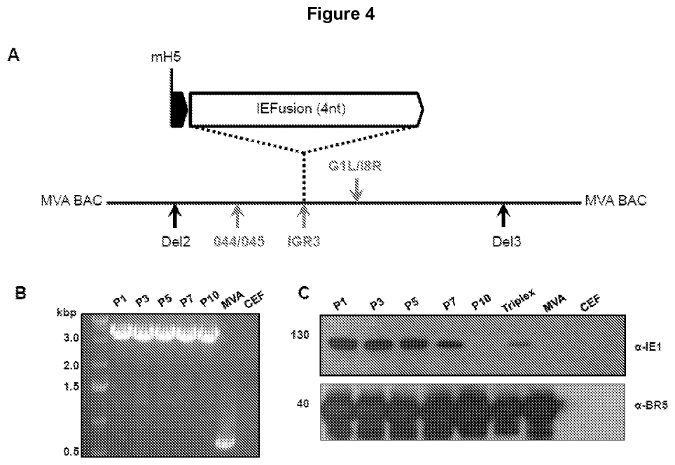

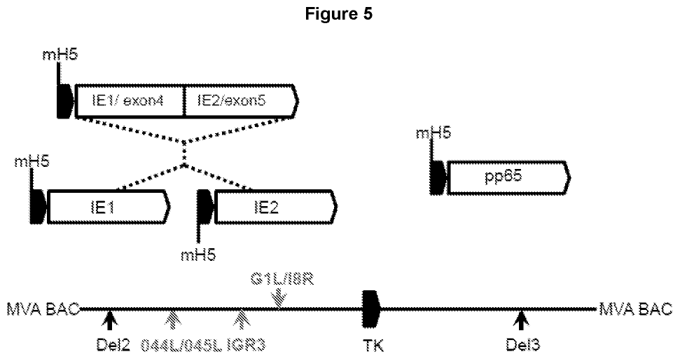

A CMV antigen can be a CMV protein antigen, a fragment of a CMV protein antigen, a modified CMV protein antigen, a fragment of a modified CMV protein antigen, a mutated CMV protein antigen or a fusion CMV protein antigen. Examples of CMV protein antigens and CMV fragments may include pp65, IE1 exon 4 (IE1/e4), IE2 exon 5 (IE2/e5), and antigenic fragments thereof. Examples of modified CMV protein antigens and fragments thereof may be found in U.S. Pat. No. 7,163,685 to Diamond et al. and is incorporated herein by reference in its entirety. Examples of mutated CMV protein antigens may be found in U.S. Pat. No. 6,835,383 to Zaia et al. and is incorporated herein by reference in its entirety. Moreover, all ranked antigens established by assessing immune response in healthy adults can be added up until reaching the maximal capacity of the MVA vector for gene insertions (see FIGS. 1C and 4D) [32].

Fusion CMV protein antigens may comprise two or more CMV proteins, modified CMV proteins, mutated CMV proteins or any antigenic fragments thereof. In one aspect, an exemplar fusion protein is a fusion of IE1 exon 4 (IE1/e4) and IE2 exon 5 (IE2/e5), IE1/e4-IE2/e5 (“IEfusion”). In one embodiment, the use of fusion proteins involves creating an IEfusion protein that comprises exon4 from IE1 and exon5 from the IE2 gene into a single gene without additional genetic material. The IEfusion protein comprises a unique representation of the immediate-early antigens than either protein alone. In another embodiment, the nucleic acid sequence encoding the IEfusion is codon optimized. In yet another embodiment, the amino acid sequence of the IEfusion protein comprises one or more His to Ala mutations in the C-terminus of IE2.

The term “genetic stability” as used herein refers to a measure of the resistance to change, with serial passage of virus, of the DNA sequence of a gene, the expression level of the gene, or both. The genetic stability of the target gene in an rMVA vector is a concern in the development of a vaccine. A reduction of the genetic stability of the target gene may have the effect of reducing the immunogenicity of the rMVA vector due to changes in gene sequence or expression level. Genetic instability of the insert gene sequence can lead to alterations of the sequence flanking the gene insertion. Suppressing the instability of the insert gene seems to curtail instability of the flanking virus DNA sequence.

Genetic stability of recombinant virus can be measured or assessed by numerous methods known in the art, e.g., testing foreign protein expression levels at each passage by Western blot (WB) or immunostaining virus plaques and calculating the percentage of foreign protein producing foci before and after serial passage. An alternative means to assess genetic stability is by real-time quantitative PCR (RT-qPCR) method, which amplifies isolated MVA genomic DNA and calculates the copy numbers of the inserted gene of interest and MVA vector after each passage. The ratio of the gene of interest copy number versus the MVA backbone vector copy number is used to determine the genetic stability of the gene or the MVA vaccine carrying the gene. A higher ratio of the gene of interest copy number to the MVA backbone vector copy number reflects a higher genetic stability, with the highest ratio=1 means approximately 100% gene expression remains after serial passage. RT-qPCR is more sensitive, high-throughput and provides highly reproducible results relative to other methods, such as Western blot or immunostaining. The method of RT-qPCR can be performed following well-known procedures in the art or the manuals of commercially available RT-qPCR kit. However, this method may not detect single nucleotide changes without accompanying sequence information. Disruptions of the coding sequence of the IE1 or IE2 inserts can prevent recognition by monoclonal antibodies that recognize intact forms.

An rMVA vaccine carrying a gene of interest is genetically stable when the DNA sequence of the gene and the expression of the gene is substantially unchanged during serial passage of the vaccine, particularly, after 10 or more passages.

Another aspect is a method for the prevention or treatment of infections or cancer in a mammal subject by administering to the subject a genetically stable rMVA vaccine disclosed herein, wherein the rMVA vaccine contains two or more CMV antigens under control of a mH5 or other poxvirus promoters, including IE1, IE2, and pp65 or antigenic fragments thereof. In some embodiments, the mammal subject is a human subject.

The nucleic acid sequences and amino acid sequences of certain IEfusions, IE proteins, and variants thereof are disclosed below.

IEfusion-VacO DNA sequence (SEQ ID NO: 1):
atggtgaagcaaatcaaggtcagagtggacatggtaagacacagaattaaggaacacatgttgaagaagtatactcaa
acagaggagaagttcaccggtgccttcaatatgatgggtggatgtctacagaacgctttggatatcttagataaggtacatg
aaccattcgaagaaatgaagtgcattggattgacaatgcaatcaatgtatgagaactacatagtgccagaggataagcgt
gaaatgtggatggcatgcatcaaggagttacatgatgtatccaaaggagcagccaacaagctaggtggtgctttgcaagc
gaaggcaagagcgaagaaggatgaattgagacgaaagatgatgtacatgtgctatcgaaacatcgaattcttcactaag
aactcagcgtttcctaagactaccaatggatgcagtcaagctatggctgcgcttcagaacttgcctcaatgtagtcctgatga
aatcatggcatatgcacagaagatcttcaagatcttagatgaggaaagagacaaggtattgactcatatcgatcacatattc
atggatatactaacaacatgtgtagaaacgatgtgtaacgagtacaaggtaacttcggacgcttgtatgatgactatgtacg
gaggaatatctctacttagtgagttctgtcgagttctatgctgttacgtattagaagaaactagtgtaatgttagcgaagagac
cattgatcactaagcctgaagtgatctcggttatgaagagacgaatagaggagatctgtatgaaggtgttcgcacaataca
tcttaggagctgatcctctaagagtgtgtagtccatcggtagacgatttgagagctatagcggaggaatctgacgaggaag
aggcaatagttgcatacacacttgctacagctggagtatccagttctgattctcttgtaagtcctccggagtcacctgtgccag
caaccataccgttgagtagtgtgattgtggctgagaactcggatcaggaagagtctgagcaatccgatgaagaagagga
ggaaggagcacaagaggagagagaagatactgtctctgtgaagagtgaacctgtatctgaaatcgaggaagtagcac
ctgaggaagaggaggatggagccgaagaaccaacagcttcgggtggtaagtcaactcatccgatggtaaccagatcta
aggcagaccagggagacatcctagcacaagcagtgaaccatgctggaattgactcatcttcgaccggaccaactctaa
cgactcattcatgttcggttagttctgctcctcttaacaagcctacacctacctcggtagctgttaccaacacacctttaccagg
agcatcagcaacacctgagttgtctccaagaaagaagcctcgtaagaccacgagaccgttcaaggtgatcatcaagcc
accagtaccacctgctccgatcatgttgccattgatcaagcaggaggacattaagccagaacctgacttcacgatacagt
accgtaacaagatcatagatacagcaggatgcatagtgatctcagatagtgaagaggagcaaggtgaggaagtggag
actagaggagccacagccagttcgccttccacaggatccggaactcctagagtaactagtccgacacatccactttccca
gatgaatcatccacctctaccggatcctctaggacgaccagatgaagattcttcttcatctagttcaagttcttgctcatccgcg
agtgatagtgagtcagaaagtgaagagatgaagtgctcttctggtggtggagctagtgtcacttcatctcatcatggacgag
gaggatttggaggtgctgcgagtagttccttactaagttgtggacatcagtcatctggtggtgcatctactggacctagaaag
aagaagtcaaagagaatctccgaattggataatgagaaagtgagaaacatcatgaaggacaagaacacgccgttctg
cactccgaatgttcagacgagaagaggacgagtgaagatagatgaagtatcacgaatgttcagaaacacaaatcgttct
ctagagtacaagaatcttccgttcaccataccttcgatgcaccaagtattagatgaggctatcaaggcatgtaagaccatgc
aagttaacaacaaaggaatacagatcatctacactagaaaccatgaggttaagagtgaggtggatgccgtacgttgtag
attgggaacgatgtgtaaccttgcgctatctactcctttcctaatggagcatactatgcctgtgactcatcctcctgaagtggct
caaagaacagctgatgcttgtaacgaaggtgtgaaagctgcttggtccctaaaggagttacatacacaccaactttgtcca
cgatccagtgactacagaaacatgatcattcatgcagctacgcctgtagatctacttggagctcttaacctatgtcttcctttga
tgcagaagttccctaagcaagtgatggtgagaatcttctcgacgaatcaaggaggattcatgttaccgatatacgagacag
ctgcaaaggcttacgctgtcggtcagttcgagcaaccgactgaaacgcctcctgaggacttagatacattgtctttggcgat
agaagcagcgattcaggatcttagaaacaagagtcagtaa
IEfusion DNA sequence (SEQ ID NO: 2):
atggtcaaacagattaaggttcgagtggacatggtgcggcatagaatcaaggagcacatgctgaaaaaatatacccag
acggaagagaaattcactggcgcctttaatatgatgggaggatgtttgcagaatgccttagatatcttagataaggttcatga
gcctttcgaggagatgaagtgtattgggctaactatgcagagcatgtatgagaactacattgtacctgaggataagcggga
gatgtggatggcttgtattaaggagctgcatgatgtgagcaagggcgccgctaacaagttggggggtgcactgcaggcta
aggcccgtgctaaaaaggatgaacttaggagaaagatgatgtatatgtgctacaggaatatagagttctttaccaagaact
cagccttccctaagaccaccaatggctgcagtcaggccatggcggcactgcagaacttgcctcagtgctcccctgatgag
attatggcttatgcccagaaaatatttaagattttggatgaggagagagacaaggtgctcacgcacattgatcacatatttat
ggatatcctcactacatgtgtggaaacaatgtgtaatgagtacaaggtcactagtgacgcttgtatgatgaccatgtacggg
ggcatctctctcttaagtgagttctgtcgggtgctgtgctgctatgtcttagaggagactagtgtgatgctggccaagcggcctc
tgataaccaagcctgaggttatcagtgtaatgaagcgccgcattgaggagatctgcatgaaggtctttgcccagtacattct
gggggccgatcctctgagagtctgctctcctagtgtggatgacctacgggccatcgccgaggagtcagatgaggaagag
gctattgtagcctacactttggccaccgctggtgtcagctcctctgattctctggtgtcacccccagagtcccctgtacccgcg
actatccctctgtcctcagtaattgtggctgagaacagtgatcaggaagaaagtgagcagagtgatgaggaagaggagg
agggtgctcaggaggagcgggaggacactgtgtctgtcaagtctgagccagtgtctgagatagaggaagttgccccaga
ggaagaggaggatggtgctgaggaacccaccgcctctggaggcaagagcacccaccctatggtgactagaagcaag
gctgaccagggtgacatcctcgcccaggctgtcaatcatgccggtatcgattccagtagcaccggccccacgctgacaac
ccactcttgcagcgttagcagcgcccctcttaacaagccgacccccaccagcgtcgcggttactaacactcctctccccgg
ggcatccgctactcccgagctcagcccgcgtaagaaaccgcgcaaaaccacgcgtcctttcaaggtgattattaaaccg
cccgtgcctcccgcgcctatcatgctgcccctcatcaaacaggaagacatcaagcccgagcccgactttaccatccagta
ccgcaacaagattatcgataccgccggctgtatcgtgatctctgatagcgaggaagaacagggtgaagaagtcgaaac
ccgcggtgctaccgcgtcttccccttccaccggcagcggcacgccgcgagtgacctctcccacgcacccgctctcccaga
tgaaccaccctcctcttcccgatcccttgggccggcccgatgaagatagttcctcttcgtcttcctcctcctgcagttcggcttcg
gactcggagagtgagtccgaggagatgaaatgcagcagtggcggaggagcatccgtgacctcgagccaccatgggc
gcggcggttttggtggcgcggcctcctcctctctgctgagctgcggccatcagagcagcggcggggcgagcaccggacc
ccgcaagaagaagagcaaacgcatctccgagttggacaacgagaaggtgcgcaatatcatgaaagataagaacacc
cccttctgcacacccaacgtgcagactcggcggggtcgcgtcaagattgacgaggtgagccgcatgttccgcaacacca
atcgctctcttgagtacaagaacctgcccttcacgattcccagtatgcaccaggtgttagatgaggccatcaaagcctgcaa
aaccatgcaggtgaacaacaagggcatccagattatctacacccgcaatcatgaggtgaagagtgaggtggatgcggt
gcggtgtcgcctgggcaccatgtgcaacctggccctctccactcccttcctcatggagcacaccatgcccgtgacacatcc
acccgaagtggcgcagcgcacagccgatgcttgtaacgaaggcgtcaaggccgcgtggagcctcaaagaattgcaca
cccaccaattatgcccccgttcctccgattaccgcaacatgatcatccacgctgccacccccgtggacctgttgggcgctct
caacctgtgcctgcccctgatgcaaaagtttcccaaacaggtcatggtgcgcatcttctccaccaaccaggggggttcatg
ctgcctatctacgagacggccgcgaaggcctacgccgtggggcagtttgagcagcccaccgagacccctcccgaagac
ctggacaccctgagcctggccatcgaggcagccatccaggacctgaggaacaagtctcagtaa
IEfusion-4 nt DNA sequence (SEQ ID NO: 3):
atggtcaaacagattaaggttcgagtggacatggtgcggcatagaatcaaggagcacatgctgaagaagtatacccag
acggaagagaaattcactggcgcctttaatatgatgggaggatgtttgcagaatgccttagatatcttagataaggttcatga
gcctttcgaggagatgaagtgtattgggctaactatgcagagcatgtatgagaactacattgtacctgaggataagcggga
gatgtggatggcttgtattaaggagctgcatgatgtgagcaagggcgccgctaacaagttaggaggtgcactgcaggcta
aggcccgtgctaagaaggatgaacttaggagaaagatgatgtatatgtgctacaggaatatagagttctttaccaagaact
cagccttccctaagaccaccaatggctgcagtcaggccatggcggcactgcagaacttgcctcagtgctctcctgatgag
attatggcttatgcccagaagatatttaagatcttggatgaggagagagacaaggtgctcacgcacattgatcacatatttat
ggatatcctcactacatgtgtggaaacaatgtgtaatgagtacaaggtcactagtgacgcttgtatgatgaccatgtacgga
ggcatctctctcttaagtgagttctgtcgggtgctgtgctgctatgtcttagaggagactagtgtgatgctggccaagcggcctc
tgataaccaagcctgaggttatcagtgtaatgaagcgccgcattgaggagatctgcatgaaggtctttgcccagtacattct
aggtgccgatcctctgagagtctgctctcctagtgtggatgacctacgggccatcgccgaggagtcagatgaggaagagg
ctattgtagcctacactttggccaccgctggtgtcagctcctctgattctctggtgtcacctccagagtcacctgtacccgcgac
tatccctctgtcctcagtaattgtggctgagaacagtgatcaggaagaaagtgagcagagtgatgaggaagaggaggag
ggtgctcaggaggagcgggaggacactgtgtctgtcaagtctgagccagtgtctgagatagaggaagttgctccagagg
aagaggaggatggtgctgaggaacccaccgcctctggaggcaagagcacccaccctatggtgactagaagcaaggct
gaccagggtgacatcctcgcccaggctgtcaatcatgccggtatcgattccagtagcaccggacctacgctgacaaccc
actcttgcagcgttagcagcgctcctcttaacaagccgactccaaccagcgtcgcggttactaacactcctctaccaggag
catccgctactcccgagctcagcccgcgtaagaaaccgcgcaagaccacgcgtcctttcaaggtgattattaaaccgccc
gtgcctcccgcgcctatcatgctgccactcatcaaacaggaagacatcaagcccgagcccgactttaccatccagtaccg
caacaagattatcgataccgccggctgtatcgtgatctctgatagcgaggaagaacagggtgaagaagtcgaaacccg
cggtgctaccgcgtcttcaccttccaccggcagcggcacgccgcgagtgacctctcccacgcacccgctctcccagatga
accaccctcctcttcccgatcccttgggccggcccgatgaagatagttcctcttcgtcttcctcctcctgcagttcggcttcgga
ctcggagagtgagtccgaggagatgaaatgcagcagtggcggaggagcatccgtgacctcgagccaccatgggcgcg
gcggatttggtggcgcggcctcctcctctctgctgagctgcggccatcagagcagcggcggtgcgagcaccggacctcg
caagaagaagagcaaacgcatctccgagttggacaacgagaaggtgcgcaatatcatgaaagataagaacactccct
tctgcacacccaacgtgcagactcggcgtggtcgcgtcaagattgacgaggtgagccgcatgttccgcaacaccaatcg
ctctcttgagtacaagaacctgcccttcacgattcccagtatgcaccaggtgttagatgaggccatcaaagcctgcaagac
catgcaggtgaacaacaagggcatccagattatctacacccgcaatcatgaggtgaagagtgaggtggatgcggtgcg
gtgtcgcctgggcaccatgtgcaacctggccctctccactcccttcctcatggagcacaccatgcccgtgacacatccacc
cgaagtggcgcagcgcacagccgatgcttgtaacgaaggcgtcaaggccgcgtggagcctcaaagaattgcacaccc
accaattatgtcctcgttcctccgattaccgcaacatgatcatccacgctgccacaccagtggacctgttgggcgctctcaac
ctgtgcctgccactgatgcagaagtttcccaaacaggtcatggtgcgcatcttctccaccaaccaggggggttcatgctgc
ctatctacgagacggccgcgaaggcctacgccgttggtcagtttgagcagcccaccgagacacctcccgaagacctgg
acaccctgagcctggccatcgaggcagccatccaggacctgaggaacaagtctcagtaa
IEfusion-VacO amino acid sequence (SEQ ID NO: 4):
MVKQIKVRVDMVRHRIKEHMLKKYTQTEEKFTGAFNMMGGCLQNALDILDKVHEPFEE
MKCIGLTMQSMYENYIVPEDKREMWMACIKELHDVSKGAANKLGGALQAKARAKKDE
LRRKMMYMCYRNIEFFTKNSAFPKTTNGCSQAMAALQNLPQCSPDEIMAYAQKIFKIL
DEERDKVLTHIDHIFMDILTTCVETMCNEYKVTSDACMMTMYGGISLLSEFCRVLCCYV
LEETSVMLAKRPLITKPEVISVMKRRIEEICMKVFAQYILGADPLRVCSPSVDDLRAIAEE
SDEEEAIVAYTLATAGVSSSDSLVSPPESPVPATIPLSSVIVAENSDQEESEQSDEEEEE
GAQEEREDTVSVKSEPVSEIEEVAPEEEEDGAEEPTASGGKSTHPMVTRSKADQGDIL
AQAVNHAGIDSSSTGPTLTTHSCSVSSAPLNKPTPTSVAVTNTPLPGASATPELSPRKK
PRKTTRPFKVIIKPPVPPAPIMLPLIKQEDIKPEPDFTIQYRNKIIDTAGCIVISDSEEEQGE
EVETRGATASSPSTGSGTPRVTSPTHPLSQMNHPPLPDPLGRPDEDSSSSSSSSCSS
ASDSESESEEMKCSSGGGASVTSSHHGRGGFGGAASSSLLSCGHQSSGGASTGPR
KKKSKRISELDNEKVRNIMKDKNTPFCTPNVQTRRGRVKIDEVSRMFRNTNRSLEYKN
LPFTIPSMHQVLDEAIKACKTMQVNNKGIQIIYTRNHEVKSEVDAVRCRLGTMCNLALS
TPFLMEHTMPVTHPPEVAQRTADACNEGVKAAWSLKELHTHQLCPRSSDYRNMIIHA
ATPVDLLGALNLCLPLMQKFPKQVMVRIFSTNQGGFMLPIYETAAKAYAVGQFEQPTE
TPPEDLDTLSLAIEAAIQDLRNKSQ
IEfusion amino acid sequence (SEQ ID NO: 5):
MVKQIKVRVDMVRHRIKEHMLKKYTQTEEKFTGAFNMMGGCLQNALDILDKVHEPFEE
MKCIGLTMQSMYENYIVPEDKREMWMACIKELHDVSKGAANKLGGALQAKARAKKDE
LRRKMMYMCYRNIEFFTKNSAFPKTTNGCSQAMAALQNLPQCSPDEIMAYAQKIFKIL
DEERDKVLTHIDHIFMDILTTCVETMCNEYKVTSDACMMTMYGGISLLSEFCRVLCCYV
LEETSVMLAKRPLITKPEVISVMKRRIEEICMKVFAQYILGADPLRVCSPSVDDLRAIAEE
SDEEEAIVAYTLATAGVSSSDSLVSPPESPVPATIPLSSVIVAENSDQEESEQSDEEEEE
GAQEEREDTVSVKSEPVSEIEEVAPEEEEDGAEEPTASGGKSTHPMVTRSKADQGDIL
AQAVNHAGIDSSSTGPTLTTHSCSVSSAPLNKPTPTSVAVTNTPLPGASATPELSPRKK
PRKTTRPFKVIIKPPVPPAPIMLPLIKQEDIKPEPDFTIQYRNKIIDTAGCIVISDSEEEQGE
EVETRGATASSPSTGSGTPRVTSPTHPLSQMNHPPLPDPLGRPDEDSSSSSSSSCSS
ASDSESESEEMKCSSGGGASVTSSHHGRGGFGGAASSSLLSCGHQSSGGASTGPR
KKKSKRISELDNEKVRNIMKDKNTPFCTPNVQTRRGRVKIDEVSRMFRNTNRSLEYKN
LPFTIPSMHQVLDEAIKACKTMQVNNKGIQIIYTRNHEVKSEVDAVRCRLGTMCNLALS
TPFLMEHTMPVTHPPEVAQRTADACNEGVKAAWSLKELHTHQLCPRSSDYRNMIIHA
ATPVDLLGALNLCLPLMQKFPKQVMVRIFSTNQGGFMLPIYETAAKAYAVGQFEQPTE
TPPEDLDTLSLAIEAAIQDLRNKSQ
IEfusion-4 NT amino acid sequence (SEQ ID NO: 6):
MVKQIKVRVDMVRHRIKEHMLKKYTQTEEKFTGAFNMMGGCLQNALDILDKVHEPFEE
MKCIGLTMQSMYENYIVPEDKREMWMACIKELHDVSKGAANKLGGALQAKARAKKDE
LRRKMMYMCYRNIEFFTKNSAFPKTTNGCSQAMAALQNLPQCSPDEIMAYAQKIFKIL
DEERDKVLTHIDHIFMDILTTCVETMCNEYKVTSDACMMTMYGGISLLSEFCRVLCCYV
LEETSVMLAKRPLITKPEVISVMKRRIEEICMKVFAQYILGADPLRVCSPSVDDLRAIAEE
SDEEEAIVAYTLATAGVSSSDSLVSPPESPVPATIPLSSVIVAENSDQEESEQSDEEEEE
GAQEEREDTVSVKSEPVSEIEEVAPEEEEDGAEEPTASGGKSTHPMVTRSKADQGDIL
AQAVNHAGIDSSSTGPTLTTHSCSVSSAPLNKPTPTSVAVTNTPLPGASATPELSPRKK
PRKTTRPFKVIIKPPVPPAPIMLPLIKQEDIKPEPDFTIQYRNKIIDTAGCIVISDSEEEQGE
EVETRGATASSPSTGSGTPRVTSPTHPLSQMNHPPLPDPLGRPDEDSSSSSSSSCSS
ASDSESESEEMKCSSGGGASVTSSHHGRGGFGGAASSSLLSCGHQSSGGASTGPR
KKKSKRISELDNEKVRNIMKDKNTPFCTPNVQTRRGRVKIDEVSRMFRNTNRSLEYKN
LPFTIPSMHQVLDEAIKACKTMQVNNKGIQIIYTRNHEVKSEVDAVRCRLGTMCNLALS
TPFLMEHTMPVTHPPEVAQRTADACNEGVKAAWSLKELHTHQLCPRSSDYRNMIIHA
ATPVDLLGALNLCLPLMQKFPKQVMVRIFSTNQGGFMLPIYETAAKAYAVGQFEQPTE
TPPEDLDTLSLAIEAAIQDLRNKSQ
IE2-VacO DNA sequence (SEQ ID NO: 7):
atgggagacatcctagcacaagcagtgaaccatgctggaattgactcatcttcgaccggaccaactctaacgactcattc
atgttcggttagttctgctcctcttaacaagcctacacctacctcggtagctgttaccaacacacctttaccaggagcatcagc
aacacctgagttgtctccaagaaagaagcctcgtaagaccacgagaccgttcaaggtgatcatcaagccaccagtacc
acctgctccgatcatgttgccattgatcaagcaggaggacattaagccagaacctgacttcacgatacagtaccgtaaca
agatcatagatacagcaggatgcatagtgatctcagatagtgaagaggagcaaggtgaggaagtggagactagagga
gccacagccagttcgccttccacaggatccggaactcctagagtaactagtccgacacatccactttcccagatgaatcat
ccacctctaccggatcctctaggacgaccagatgaagattcttcttcatctagttcaagttcttgctcatccgcgagtgatagtg
agtcagaaagtgaagagatgaagtgctcttctggtggtggagctagtgtcacttcatctcatcatggacgaggaggatttgg
aggtgctgcgagtagttccttactaagttgtggacatcagtcatctggtggtgcatctactggacctagaaagaagaagtca
aagagaatctccgaattggataatgagaaagtgagaaacatcatgaaggacaagaacacgccgttctgcactccgaat
gttcagacgagaagaggacgagtgaagatagatgaagtatcacgaatgttcagaaacacaaatcgttctctagagtaca
agaatcttccgttcaccataccttcgatgcaccaagtattagatgaggctatcaaggcatgtaagaccatgcaagttaacaa
caaaggaatacagatcatctacactagaaaccatgaggttaagagtgaggtggatgccgtacgttgtagattgggaacg
atgtgtaaccttgcgctatctactcctttcctaatggagcatactatgcctgtgactcatcctcctgaagtggctcaaagaaca
gctgatgcttgtaacgaaggtgtgaaagctgcttggtccctaaaggagttacatacacaccaactttgtccacgatccagtg
actacagaaacatgatcattcatgcagctacgcctgtagatctacttggagctcttaacctatgtcttcctttgatgcagaagtt
ccctaagcaagtgatggtgagaatcttctcgacgaatcaaggaggattcatgttaccgatatacgagacagctgcaaagg
cttacgctgtcggtcagttcgagcaaccgactgaaacgcctcctgaggacttagatacattgtctttggcgatagaagcagc
gattcaggatcttagaaacaagagtcagtaa
IE2 DNA sequence (SEQ ID NO: 8):
atgggtgacatcctcgcccaggctgtcaatcatgccggtatcgattccagtagcaccggccccacgctgacaacccactct
tgcagcgttagcagcgcccctcttaacaagccgacccccaccagcgtcgcggttactaacactcctctccccggggcatc
cgctactcccgagctcagcccgcgtaagaaaccgcgcaaaaccacgcgtcctttcaaggtgattattaaaccgcccgtgc
ctcccgcgcctatcatgctgcccctcatcaaacaggaagacatcaagcccgagcccgactttaccatccagtaccgcaa
caagattatcgataccgccggctgtatcgtgatctctgatagcgaggaagaacagggtgaagaagtcgaaacccgcggt
gctaccgcgtcttccccttccaccggcagcggcacgccgcgagtgacctctcccacgcacccgctctcccagatgaacc
accctcctcttcccgatcccttgggccggcccgatgaagatagttcctcttcgtcttcctcctcctgcagttcggcttcggactcg
gagagtgagtccgaggagatgaaatgcagcagtggcggaggagcatccgtgacctcgagccaccatgggcgcggcg
gttttggtggcgcggcctcctcctctctgctgagctgcggccatcagagcagcggcggggcgagcaccggaccccgcaa
gaagaagagcaaacgcatctccgagttggacaacgagaaggtgcgcaatatcatgaaagataagaacacccccttct
gcacacccaacgtgcagactcggcggggtcgcgtcaagattgacgaggtgagccgcatgttccgcaacaccaatcgct
ctcttgagtacaagaacctgcccttcacgattcccagtatgcaccaggtgttagatgaggccatcaaagcctgcaaaacca
tgcaggtgaacaacaagggcatccagattatctacacccgcaatcatgaggtgaagagtgaggtggatgcggtgcggtg
tcgcctgggcaccatgtgcaacctggccctctccactcccttcctcatggagcacaccatgcccgtgacacatccacccga
agtggcgcagcgcacagccgatgcttgtaacgaaggcgtcaaggccgcgtggagcctcaaagaattgcacacccacc
aattatgcccccgttcctccgattaccgcaacatgatcatccacgctgccacccccgtggacctgttgggcgctctcaacct
gtgcctgcccctgatgcaaaagtttcccaaacaggtcatggtgcgcatcttctccaccaaccaggggggttcatgctgcct
atctacgagacggccgcgaaggcctacgccgtggggcagtttgagcagcccaccgagacccctcccgaagacctgga
caccctg agcctggccatcgaggcagccatccaggacctgaggaacaagtctcagtaa
IE2-4 nt DNA sequence (SEQ ID NO: 9):
atgggtgacatcctcgcccaggctgtcaatcatgccggtatcgattccagtagcaccggacctacgctgacaacccactct
tgcagcgttagcagcgctcctcttaacaagccgactccaaccagcgtcgcggttactaacactcctctaccaggagcatcc
gctactcccgagctcagcccgcgtaagaaaccgcgcaagaccacgcgtcctttcaaggtgattattaaaccgcccgtgcc
tcccgcgcctatcatgctgccactcatcaaacaggaagacatcaagcccgagcccgactttaccatccagtaccgcaac
aagattatcgataccgccggctgtatcgtgatctctgatagcgaggaagaacagggtgaagaagtcgaaacccgcggtg
ctaccgcgtcttcaccttccaccggcagcggcacgccgcgagtgacctctcccacgcacccgctctcccagatgaacca
ccctcctcttcccgatcccttgggccggcccgatgaagatagttcctcttcgtcttcctcctcctgcagttcggcttcggactcgg
agagtgagtccgaggagatgaaatgcagcagtggcggaggagcatccgtgacctcgagccaccatgggcgcggcgg
atttggtggcgcggcctcctcctctctgctgagctgcggccatcagagcagcggcggtgcgagcaccggacctcgcaag
aagaagagcaaacgcatctccgagttggacaacgagaaggtgcgcaatatcatgaaagataagaacactcccttctgc
acacccaacgtgcagactcggcgtggtcgcgtcaagattgacgaggtgagccgcatgttccgcaacaccaatcgctctct
tgagtacaagaacctgcccttcacgattcccagtatgcaccaggtgttagatgaggccatcaaagcctgcaagaccatgc
aggtgaacaacaagggcatccagattatctacacccgcaatcatgaggtgaagagtgaggtggatgcggtgcggtgtcg
cctgggcaccatgtgcaacctggccctctccactcccttcctcatggagcacaccatgcccgtgacacatccacccgaagt
ggcgcagcgcacagccgatgcttgtaacgaaggcgtcaaggccgcgtggagcctcaaagaattgcacacccaccaatt
atgtcctcgttcctccgattaccgcaacatgatcatccacgctgccacaccagtggacctgttgggcgctctcaacctgtgcc
tgccactgatgcagaagtttcccaaacaggtcatggtgcgcatcttctccaccaaccagggtgggttcatgctgcctatctac
gagacggccgcgaaggcctacgccgttggtcagtttgagcagcccaccgagacacctcccgaagacctggacaccct
gagcctggccatcgaggcagccatccaggacctgaggaacaagtctcagtaa
IE2 4 nt H363A DNA sequence (mutations are shown in bold and
underlined)(SEQ ID NO: 10):
atgggtgacatcctcgcccaggctgtcaatcatgccggtatcgattccagtagcaccggacctacgctgacaacccactct
tgcagcgttagcagcgctcctcttaacaagccgactccaaccagcgtcgcggttactaacactcctctaccaggagcatcc
gctactcccgagctcagcccgcgtaagaaaccgcgcaagaccacgcgtcctttcaaggtgattattaaaccgcccgtgcc
tcccgcgcctatcatgctgccactcatcaaacaggaagacatcaagcccgagcccgactttaccatccagtaccgcaac
aagattatcgataccgccggctgtatcgtgatctctgatagcgaggaagaacagggtgaagaagtcgaaacccgcggtg
ctaccgcgtcttcaccttccaccggcagcggcacgccgcgagtgacctctcccacgcacccgctctcccagatgaacca
ccctcctcttcccgatcccttgggccggcccgatgaagatagttcctcttcgtcttcctcctcctgcagttcggcttcggactcgg
agagtgagtccgaggagatgaaatgcagcagtggcggaggagcatccgtgacctcgagccaccatgggcgcggcgg
atttggtggcgcggcctcctcctctctgctgagctgcggccatcagagcagcggcggtgcgagcaccggacctcgcaag
aagaagagcaaacgcatctccgagttggacaacgagaaggtgcgcaatatcatgaaagataagaacactcccttctgc
acacccaacgtgcagactcggcgtggtcgcgtcaagattgacgaggtgagccgcatgttccgcaacaccaatcgctctct
tgagtacaagaacctgcccttcacgattcccagtatgcaccaggtgttagatgaggccatcaaagcctgcaagaccatgc
aggtgaacaacaagggcatccagattatctacacccgcaatcatgaggtgaagagtgaggtggatgcggtgcggtgtcg
cctgggcaccatgtgcaacctggccctctccactcccttcctcatggaggcaaccatgcccgtgacacatccacccgaag
tggcgcagcgcacagccgatgcttgtaacgaaggcgtcaaggccgcgtggagcctcaaagaattgcacacccaccaat
tatgtcctcgttcctccgattaccgcaacatgatcatccacgctgccacaccagtggacctgttgggcgctctcaacctgtgc
ctgccactgatgcagaagtttcccaaacaggtcatggtgcgcatcttctccaccaaccaggggggttcatgctgcctatcta
cgagacggccgcgaaggcctacgccgttggtcagtttgagcagcccaccgagacacctcccgaagacctggacaccct
gagcctggccatcgaggcagccatccaggacctgaggaacaagtctcagtaa
IE2 4 nt H369A DNA sequence (mutations are shown in bold and
underlined)(SEQ ID NO: 11):
atgggtgacatcctcgcccaggctgtcaatcatgccggtatcgattccagtagcaccggacctacgctgacaacccactct
tgcagcgttagcagcgctcctcttaacaagccgactccaaccagcgtcgcggttactaacactcctctaccaggagcatcc
gctactcccgagctcagcccgcgtaagaaaccgcgcaagaccacgcgtcctttcaaggtgattattaaaccgcccgtgcc
tcccgcgcctatcatgctgccactcatcaaacaggaagacatcaagcccgagcccgactttaccatccagtaccgcaac
aagattatcgataccgccggctgtatcgtgatctctgatagcgaggaagaacagggtgaagaagtcgaaacccgcggtg
ctaccgcgtcttcaccttccaccggcagcggcacgccgcgagtgacctctcccacgcacccgctctcccagatgaacca
ccctcctcttcccgatcccttgggccggcccgatgaagatagttcctcttcgtcttcctcctcctgcagttcggcttcggactcgg
agagtgagtccgaggagatgaaatgcagcagtggcggaggagcatccgtgacctcgagccaccatgggcgcggcgg
atttggtggcgcggcctcctcctctctgctgagctgcggccatcagagcagcggcggtgcgagcaccggacctcgcaag
aagaagagcaaacgcatctccgagttggacaacgagaaggtgcgcaatatcatgaaagataagaacactcccttctgc
acacccaacgtgcagactcggcgtggtcgcgtcaagattgacgaggtgagccgcatgttccgcaacaccaatcgctctct
tgagtacaagaacctgcccttcacgattcccagtatgcaccaggtgttagatgaggccatcaaagcctgcaagaccatgc
aggtgaacaacaagggcatccagattatctacacccgcaatcatgaggtgaagagtgaggtggatgcggtgcggtgtcg
cctgggcaccatgtgcaacctggccctctccactcccttcctcatggagcacaccatgcccgtgacagcaccacccgaa
gtggcgcagcgcacagccgatgcttgtaacgaaggcgtcaaggccgcgtggagcctcaaagaattgcacacccacca
attatgtcctcgttcctccgattaccgcaacatgatcatccacgctgccacaccagtggacctgttgggcgctctcaacctgtg
cctgccactgatgcagaagtttcccaaacaggtcatggtgcgcatcttctccaccaaccagggtgggttcatgctgcctatct
acgagacggccgcgaaggcctacgccgttggtcagtttgagcagcccaccgagacacctcccgaagacctggacacc
ctgagcctggccatcgaggcagccatccaggacctgaggaacaagtctcagtaa
IE2 4 nt H363A/H369A DNA sequence (mutations are shown in bold and
underlined)(SEQ ID NO: 12):
atgggtgacatcctcgcccaggctgtcaatcatgccggtatcgattccagtagcaccggacctacgctgacaacccactct
tgcagcgttagcagcgctcctcttaacaagccgactccaaccagcgtcgcggttactaacactcctctaccaggagcatcc
gctactcccgagctcagcccgcgtaagaaaccgcgcaagaccacgcgtcctttcaaggtgattattaaaccgcccgtgcc
tcccgcgcctatcatgctgccactcatcaaacaggaagacatcaagcccgagcccgactttaccatccagtaccgcaac
aagattatcgataccgccggctgtatcgtgatctctgatagcgaggaagaacagggtgaagaagtcgaaacccgcggtg
ctaccgcgtcttcaccttccaccggcagcggcacgccgcgagtgacctctcccacgcacccgctctcccagatgaacca
ccctcctcttcccgatcccttgggccggcccgatgaagatagttcctcttcgtcttcctcctcctgcagttcggcttcggactcgg
agagtgagtccgaggagatgaaatgcagcagtggcggaggagcatccgtgacctcgagccaccatgggcgcggcgg
atttggtggcgcggcctcctcctctctgctgagctgcggccatcagagcagcggcggtgcgagcaccggacctcgcaag
aagaagagcaaacgcatctccgagttggacaacgagaaggtgcgcaatatcatgaaagataagaacactcccttctgc
acacccaacgtgcagactcggcgtggtcgcgtcaagattgacgaggtgagccgcatgttccgcaacaccaatcgctctct
tgagtacaagaacctgcccttcacgattcccagtatgcaccaggtgttagatgaggccatcaaagcctgcaagaccatgc
aggtgaacaacaagggcatccagattatctacacccgcaatcatgaggtgaagagtgaggtggatgcggtgcggtgtcg
cctgggcaccatgtgcaacctggccctctccactcccttcctcatggaggcaaccatgcccgtgacagcaccacccgaa
gtggcgcagcgcacagccgatgcttgtaacgaaggcgtcaaggccgcgtggagcctcaaagaattgcacacccacca
attatgtcctcgttcctccgattaccgcaacatgatcatccacgctgccacaccagtggacctgttgggcgctctcaacctgtg
cctgccactgatgcagaagtttcccaaacaggtcatggtgcgcatcttctccaccaaccaggggggttcatgctgcctatct
acgagacggccgcgaaggcctacgccgttggtcagtttgagcagcccaccgagacacctcccgaagacctggacacc
ctgagcctggccatcgaggcagccatccaggacctgaggaacaagtctcagtaa
IE2 H363A DNA sequence (mutations are shown in bold and underlined)
(SEQ ID NO: 13):
atgggtgacatcctcgcccaggctgtcaatcatgccggtatcgattccagtagcaccggccccacgctgacaacccactct
tgcagcgttagcagcgcccctcttaacaagccgacccccaccagcgtcgcggttactaacactcctctccccggggcatc
cgctactcccgagctcagcccgcgtaagaaaccgcgcaaaaccacgcgtcctttcaaggtgattattaaaccgcccgtgc
ctcccgcgcctatcatgctgcccctcatcaaacaggaagacatcaagcccgagcccgactttaccatccagtaccgcaa
caagattatcgataccgccggctgtatcgtgatctctgatagcgaggaagaacagggtgaagaagtcgaaacccgcggt
gctaccgcgtcttccccttccaccggcagcggcacgccgcgagtgacctctcccacgcacccgctctcccagatgaacc
accctcctcttcccgatcccttgggccggcccgatgaagatagttcctcttcgtcttcctcctcctgcagttcggcttcggactcg
gagagtgagtccgaggagatgaaatgcagcagtggcggaggagcatccgtgacctcgagccaccatgggcgcggcg
gttttggtggcgcggcctcctcctctctgctgagctgcggccatcagagcagcggcggggcgagcaccggaccccgcaa
gaagaagagcaaacgcatctccgagttggacaacgagaaggtgcgcaatatcatgaaagataagaacacccccttct
gcacacccaacgtgcagactcggcggggtcgcgtcaagattgacgaggtgagccgcatgttccgcaacaccaatcgct
ctcttgagtacaagaacctgcccttcacgattcccagtatgcaccaggtgttagatgaggccatcaaagcctgcaaaacca
tgcaggtgaacaacaagggcatccagattatctacacccgcaatcatgaggtgaagagtgaggtggatgcggtgcggtg
tcgcctgggcaccatgtgcaacctggccctctccactcccttcctcatggaggcaaccatgcccgtgacacatccacccg
aagtggcgcagcgcacagccgatgcttgtaacgaaggcgtcaaggccgcgtggagcctcaaagaattgcacacccac
caattatgcccccgttcctccgattaccgcaacatgatcatccacgctgccacccccgtggacctgttgggcgctctcaacct
gtgcctgcccctgatgcaaaagtttcccaaacaggtcatggtgcgcatcttctccaccaaccaggggggttcatgctgcct
atctacgagacggccgcgaaggcctacgccgtggggcagtttgagcagcccaccgagacccctcccgaagacctgga
caccctgagcctggccatcgaggcagccatccaggacctgaggaacaagtctcag
IE2 H369A DNA sequence (mutations are shown in bold and underlined)
(SEQ ID NO: 14):
atgggtgacatcctcgcccaggctgtcaatcatgccggtatcgattccagtagcaccggccccacgctgacaacccactct
tgcagcgttagcagcgcccctcttaacaagccgacccccaccagcgtcgcggttactaacactcctctccccggggcatc
cgctactcccgagctcagcccgcgtaagaaaccgcgcaaaaccacgcgtcctttcaaggtgattattaaaccgcccgtgc
ctcccgcgcctatcatgctgcccctcatcaaacaggaagacatcaagcccgagcccgactttaccatccagtaccgcaa
caagattatcgataccgccggctgtatcgtgatctctgatagcgaggaagaacagggtgaagaagtcgaaacccgcggt
gctaccgcgtcttccccttccaccggcagcggcacgccgcgagtgacctctcccacgcacccgctctcccagatgaacc
accctcctcttcccgatcccttgggccggcccgatgaagatagttcctcttcgtcttcctcctcctgcagttcggcttcggactcg
gagagtgagtccgaggagatgaaatgcagcagtggcggaggagcatccgtgacctcgagccaccatgggcgcggcg
gttttggtggcgcggcctcctcctctctgctgagctgcggccatcagagcagcggcggggcgagcaccggaccccgcaa
gaagaagagcaaacgcatctccgagttggacaacgagaaggtgcgcaatatcatgaaagataagaacacccccttct
gcacacccaacgtgcagactcggcggggtcgcgtcaagattgacgaggtgagccgcatgttccgcaacaccaatcgct
ctcttgagtacaagaacctgcccttcacgattcccagtatgcaccaggtgttagatgaggccatcaaagcctgcaaaacca
tgcaggtgaacaacaagggcatccagattatctacacccgcaatcatgaggtgaagagtgaggtggatgcggtgcggtg
tcgcctgggcaccatgtgcaacctggccctctccactcccttcctcatggagcacaccatgcccgtgacagcaccacccg
aagtggcgcagcgcacagccgatgcttgtaacgaaggcgtcaaggccgcgtggagcctcaaagaattgcacacccac
caattatgcccccgttcctccgattaccgcaacatgatcatccacgctgccacccccgtggacctgttgggcgctctcaacct
gtgcctgcccctgatgcaaaagtttcccaaacaggtcatggtgcgcatcttctccaccaaccagggtgggttcatgctgcct
atctacgagacggccgcgaaggcctacgccgtggggcagtttgagcagcccaccgagacccctcccgaagacctgga
caccctgagcctggccatcgaggcagccatccaggacctgaggaacaagtctcag
IE2 H363A/H369A DNA sequence (mutations are shown in bold and
underlined)(SEQ ID NO: 15):
atgggtgacatcctcgcccaggctgtcaatcatgccggtatcgattccagtagcaccggccccacgctgacaacccactct
tgcagcgttagcagcgcccctcttaacaagccgacccccaccagcgtcgcggttactaacactcctctccccggggcatc
cgctactcccgagctcagcccgcgtaagaaaccgcgcaaaaccacgcgtcctttcaaggtgattattaaaccgcccgtgc
ctcccgcgcctatcatgctgcccctcatcaaacaggaagacatcaagcccgagcccgactttaccatccagtaccgcaa
caagattatcgataccgccggctgtatcgtgatctctgatagcgaggaagaacagggtgaagaagtcgaaacccgcggt
gctaccgcgtcttccccttccaccggcagcggcacgccgcgagtgacctctcccacgcacccgctctcccagatgaacc
accctcctcttcccgatcccttgggccggcccgatgaagatagttcctcttcgtcttcctcctcctgcagttcggcttcggactcg
gagagtgagtccgaggagatgaaatgcagcagtggcggaggagcatccgtgacctcgagccaccatgggcgcggcg
gttttggtggcgcggcctcctcctctctgctgagctgcggccatcagagcagcggcggggcgagcaccggaccccgcaa
gaagaagagcaaacgcatctccgagttggacaacgagaaggtgcgcaatatcatgaaagataagaacacccccttct
gcacacccaacgtgcagactcggcggggtcgcgtcaagattgacgaggtgagccgcatgttccgcaacaccaatcgct
ctcttgagtacaagaacctgcccttcacgattcccagtatgcaccaggtgttagatgaggccatcaaagcctgcaaaacca
tgcaggtgaacaacaagggcatccagattatctacacccgcaatcatgaggtgaagagtgaggtggatgcggtgcggtg
tcgcctgggcaccatgtgcaacctggccctctccactcccttcctcatggaggcaaccatgcccgtgacagcaccacccg
aagtggcgcagcgcacagccgatgcttgtaacgaaggcgtcaaggccgcgtggagcctcaaagaattgcacacccac
caattatgcccccgttcctccgattaccgcaacatgatcatccacgctgccacccccgtggacctgttgggcgctctcaacct
gtgcctgcccctgatgcaaaagtttcccaaacaggtcatggtgcgcatcttctccaccaaccaggggggttcatgctgcct
atctacgagacggccgcgaaggcctacgccgtggggcagtttgagcagcccaccgagacccctcccgaagacctgga
caccctgagcctggccatcgaggcagccatccaggacctgaggaacaagtctcag
IE2 VacO H363A DNA sequence (mutations are shown in bold and
underlined)(SEQ ID NO: 16):
atgggagacatcctagcacaagcagtgaaccatgctggaattgactcatcttcgaccggaccaactctaacgactcattc
atgttcggttagttctgctcctcttaacaagcctacacctacctcggtagctgttaccaacacacctttaccaggagcatcagc
aacacctgagttgtctccaagaaagaagcctcgtaagaccacgagaccgttcaaggtgatcatcaagccaccagtacc
acctgctccgatcatgttgccattgatcaagcaggaggacattaagccagaacctgacttcacgatacagtaccgtaaca
agatcatagatacagcaggatgcatagtgatctcagatagtgaagaggagcaaggtgaggaagtggagactagagga
gccacagccagttcgccttccacaggatccggaactcctagagtaactagtccgacacatccactttcccagatgaatcat
ccacctctaccggatcctctaggacgaccagatgaagattcttcttcatctagttcaagttcttgctcatccgcgagtgatagtg
agtcagaaagtgaagagatgaagtgctcttctggtggtggagctagtgtcacttcatctcatcatggacgaggaggatttgg
aggtgctgcgagtagttccttactaagttgtggacatcagtcatctggtggtgcatctactggacctagaaagaagaagtca
aagagaatctccgaattggataatgagaaagtgagaaacatcatgaaggacaagaacacgccgttctgcactccgaat
gttcagacgagaagaggacgagtgaagatagatgaagtatcacgaatgttcagaaacacaaatcgttctctagagtaca
agaatcttccgttcaccataccttcgatgcaccaagtattagatgaggctatcaaggcatgtaagaccatgcaagttaacaa
caaaggaatacagatcatctacactagaaaccatgaggttaagagtgaggtggatgccgtacgttgtagattgggaacg
atgtgtaaccttgcgctatctactcctttcctaatggaggctactatgcctgtgactcatcctcctgaagtggctcaaagaaca
gctgatgcttgtaacgaaggtgtgaaagctgcttggtccctaaaggagttacatacacaccaactttgtccacgatccagtg
actacagaaacatgatcattcatgcagctacgcctgtagatctacttggagctcttaacctatgtcttcctttgatgcagaagtt
ccctaagcaagtgatggtgagaatcttctcgacgaatcaaggaggattcatgttaccgatatacgagacagctgcaaagg
cttacgctgtcggtcagttcgagcaaccgactgaaacgcctcctgaggacttagatacattgtctttggcgatagaagcagc
gattcaggatcttagaaacaagagtcag
IE2 VacO H369A DNA sequence (mutations are shown in bold and
underlined)(SEQ ID NO: 17):
atgggagacatcctagcacaagcagtgaaccatgctggaattgactcatcttcgaccggaccaactctaacgactcattc
atgttcggttagttctgctcctcttaacaagcctacacctacctcggtagctgttaccaacacacctttaccaggagcatcagc
aacacctgagttgtctccaagaaagaagcctcgtaagaccacgagaccgttcaaggtgatcatcaagccaccagtacc
acctgctccgatcatgttgccattgatcaagcaggaggacattaagccagaacctgacttcacgatacagtaccgtaaca
agatcatagatacagcaggatgcatagtgatctcagatagtgaagaggagcaaggtgaggaagtggagactagagga
gccacagccagttcgccttccacaggatccggaactcctagagtaactagtccgacacatccactttcccagatgaatcat
ccacctctaccggatcctctaggacgaccagatgaagattcttcttcatctagttcaagttcttgctcatccgcgagtgatagtg
agtcagaaagtgaagagatgaagtgctcttctggtggtggagctagtgtcacttcatctcatcatggacgaggaggatttgg
aggtgctgcgagtagttccttactaagttgtggacatcagtcatctggtggtgcatctactggacctagaaagaagaagtca
aagagaatctccgaattggataatgagaaagtgagaaacatcatgaaggacaagaacacgccgttctgcactccgaat
gttcagacgagaagaggacgagtgaagatagatgaagtatcacgaatgttcagaaacacaaatcgttctctagagtaca
agaatcttccgttcaccataccttcgatgcaccaagtattagatgaggctatcaaggcatgtaagaccatgcaagttaacaa
caaaggaatacagatcatctacactagaaaccatgaggttaagagtgaggtggatgccgtacgttgtagattgggaacg
atgtgtaaccttgcgctatctactcctttcctaatggagcatactatgcctgtgactgctcctcctgaagtggctcaaagaaca
gctgatgcttgtaacgaaggtgtgaaagctgcttggtccctaaaggagttacatacacaccaactttgtccacgatccagtg
actacagaaacatgatcattcatgcagctacgcctgtagatctacttggagctcttaacctatgtcttcctttgatgcagaagtt
ccctaagcaagtgatggtgagaatcttctcgacgaatcaaggaggattcatgttaccgatatacgagacagctgcaaagg
cttacgctgtcggtcagttcgagcaaccgactgaaacgcctcctgaggacttagatacattgtctttggcgatagaagcagc
gattcaggatcttagaaacaagagtcag
IE2 VacO H363A/H369A DNA sequence (mutations are shown in bold and
underlined)(SEQ ID NO: 18):
atgggagacatcctagcacaagcagtgaaccatgctggaattgactcatcttcgaccggaccaactctaacgactcattc
atgttcggttagttctgctcctcttaacaagcctacacctacctcggtagctgttaccaacacacctttaccaggagcatcagc
aacacctgagttgtctccaagaaagaagcctcgtaagaccacgagaccgttcaaggtgatcatcaagccaccagtacc
acctgctccgatcatgttgccattgatcaagcaggaggacattaagccagaacctgacttcacgatacagtaccgtaaca
agatcatagatacagcaggatgcatagtgatctcagatagtgaagaggagcaaggtgaggaagtggagactagagga
gccacagccagttcgccttccacaggatccggaactcctagagtaactagtccgacacatccactttcccagatgaatcat
ccacctctaccggatcctctaggacgaccagatgaagattcttcttcatctagttcaagttcttgctcatccgcgagtgatagtg
agtcagaaagtgaagagatgaagtgctcttctggtggtggagctagtgtcacttcatctcatcatggacgaggaggatttgg
aggtgctgcgagtagttccttactaagttgtggacatcagtcatctggtggtgcatctactggacctagaaagaagaagtca
aagagaatctccgaattggataatgagaaagtgagaaacatcatgaaggacaagaacacgccgttctgcactccgaat
gttcagacgagaagaggacgagtgaagatagatgaagtatcacgaatgttcagaaacacaaatcgttctctagagtaca
agaatcttccgttcaccataccttcgatgcaccaagtattagatgaggctatcaaggcatgtaagaccatgcaagttaacaa
caaaggaatacagatcatctacactagaaaccatgaggttaagagtgaggtggatgccgtacgttgtagattgggaacg
atgtgtaaccttgcgctatctactcctttcctaatggaggctactatgcctgtgactgctcctcctgaagtggctcaaagaaca
gctgatgcttgtaacgaaggtgtgaaagctgcttggtccctaaaggagttacatacacaccaactttgtccacgatccagtg
actacagaaacatgatcattcatgcagctacgcctgtagatctacttggagctcttaacctatgtcttcctttgatgcagaagtt
ccctaagcaagtgatggtgagaatcttctcgacgaatcaaggaggattcatgttaccgatatacgagacagctgcaaagg
cttacgctgtcggtcagttcgagcaaccgactgaaacgcctcctgaggacttagatacattgtctttggcgatagaagcagc
gattcaggatcttagaaacaagagtcag
IE2-VacO amino acid sequence (SEQ ID NO: 19):
MGDILAQAVNHAGIDSSSTGPTLTTHSCSVSSAPLNKPTPTSVAVTNTPLPGASATPEL
SPRKKPRKTTRPFKVIIKPPVPPAPIMLPLIKQEDIKPEPDFTIQYRNKIIDTAGCIVISDSE
EEQGEEVETRGATASSPSTGSGTPRVTSPTHPLSQMNHPPLPDPLGRPDEDSSSSSS
SSCSSASDSESESEEMKCSSGGGASVTSSHHGRGGFGGAASSSLLSCGHQSSGGAS
TGPRKKKSKRISELDNEKVRNIMKDKNTPFCTPNVQTRRGRVKIDEVSRMFRNTNRSL
EYKNLPFTIPSMHQVLDEAIKACKTMQVNNKGIQIIYTRNHEVKSEVDAVRCRLGTMCN
LALSTPFLMEHTMPVTHPPEVAQRTADACNEGVKAAWSLKELHTHQLCPRSSDYRNM
IIHAATPVDLLGALNLCLPLMQKFPKQVMVRIFSTNQGGFMLPIYETAAKAYAVGQFEQ
PTETPPEDLDTLSLAIEAAIQDLRNKSQ
IE2 amino acid sequence (SEQ ID NO: 20):
MGDILAQAVNHAGIDSSSTGPTLTTHSCSVSSAPLNKPTPTSVAVTNTPLPGASATPEL
SPRKKPRKTTRPFKVIIKPPVPPAPIMLPLIKQEDIKPEPDFTIQYRNKIIDTAGCIVISDSE
EEQGEEVETRGATASSPSTGSGTPRVTSPTHPLSQMNHPPLPDPLGRPDEDSSSSSS
SSCSSASDSESESEEMKCSSGGGASVTSSHHGRGGFGGAASSSLLSCGHQSSGGAS
TGPRKKKSKRISELDNEKVRNIMKDKNTPFCTPNVQTRRGRVKIDEVSRMFRNTNRSL
EYKNLPFTIPSMHQVLDEAIKACKTMQVNNKGIQIIYTRNHEVKSEVDAVRCRLGTMCN
LALSTPFLMEHTMPVTHPPEVAQRTADACNEGVKAAWSLKELHTHQLCPRSSDYRNM
IIHAATPVDLLGALNLCLPLMQKFPKQVMVRIFSTNQGGFMLPIYETAAKAYAVGQFEQ
PTETPPEDLDTLSLAIEAAIQDLRNKSQ
IE2-4 nt amino acid sequence (SEQ ID NO: 21):
MGDILAQAVNHAGIDSSSTGPTLTTHSCSVSSAPLNKPTPTSVAVTNTPLPGASATPEL
SPRKKPRKTTRPFKVIIKPPVPPAPIMLPLIKQEDIKPEPDFTIQYRNKIIDTAGCIVISDSE
EEQGEEVETRGATASSPSTGSGTPRVTSPTHPLSQMNHPPLPDPLGRPDEDSSSSSS
SSCSSASDSESESEEMKCSSGGGASVTSSHHGRGGFGGAASSSLLSCGHQSSGGAS
TGPRKKKSKRISELDNEKVRNIMKDKNTPFCTPNVQTRRGRVKIDEVSRMFRNTNRSL
EYKNLPFTIPSMHQVLDEAIKACKTMQVNNKGIQIIYTRNHEVKSEVDAVRCRLGTMCN
LALSTPFLMEHTMPVTHPPEVAQRTADACNEGVKAAWSLKELHTHQLCPRSSDYRNM
IIHAATPVDLLGALNLCLPLMQKFPKQVMVRIFSTNQGGFMLPIYETAAKAYAVGQFEQ
PTETPPEDLDTLSLAIEAAIQDLRNKSQ
IE2 H363A amino acid sequence (mutation(s) are shown in bold and
underlined)(SEQ ID NO: 22):
MGDILAQAVNHAGIDSSSTGPTLTTHSCSVSSAPLNKPTPTSVAVTNTPLPGASATPEL
SPRKKPRKTTRPFKVIIKPPVPPAPIMLPLIKQEDIKPEPDFTIQYRNKIIDTAGCIVISDSE
EEQGEEVETRGATASSPSTGSGTPRVTSPTHPLSQMNHPPLPDPLGRPDEDSSSSSS
SSCSSASDSESESEEMKCSSGGGASVTSSHHGRGGFGGAASSSLLSCGHQSSGGAS
TGPRKKKSKRISELDNEKVRNIMKDKNTPFCTPNVQTRRGRVKIDEVSRMFRNTNRSL
EYKNLPFTIPSMHQVLDEAIKACKTMQVNNKGIQIIYTRNHEVKSEVDAVRCRLGTMCN
LALSTPFLMEATMPVTHPPEVAQRTADACNEGVKAAWSLKELHTHQLCPRSSDYRNM
IIHAATPVDLLGALNLCLPLMQKFPKQVMVRIFSTNQGGFMLPIYETAAKAYAVGQFEQ
PTETPPEDLDTLSLAIEAAIQDLRNKSQ
IE2 H369A amino acid sequence (mutation(s) are shown in bold and
underlined)(SEQ ID NO: 23):
MGDILAQAVNHAGIDSSSTGPTLTTHSCSVSSAPLNKPTPTSVAVTNTPLPGASATPEL
SPRKKPRKTTRPFKVIIKPPVPPAPIMLPLIKQEDIKPEPDFTIQYRNKIIDTAGCIVISDSE
EEQGEEVETRGATASSPSTGSGTPRVTSPTHPLSQMNHPPLPDPLGRPDEDSSSSSS
SSCSSASDSESESEEMKCSSGGGASVTSSHHGRGGFGGAASSSLLSCGHQSSGGAS
TGPRKKKSKRISELDNEKVRNIMKDKNTPFCTPNVQTRRGRVKIDEVSRMFRNTNRSL
EYKNLPFTIPSMHQVLDEAIKACKTMQVNNKGIQIIYTRNHEVKSEVDAVRCRLGTMCN
LALSTPFLMEHTMPVTAPPEVAQRTADACNEGVKAAWSLKELHTHQLCPRSSDYRNM
IIHAATPVDLLGALNLCLPLMQKFPKQVMVRIFSTNQGGFMLPIYETAAKAYAVGQFEQ
PTETPPEDLDTLSLAIEAAIQDLRNKSQ
IE2 H363A/H369A amino acid sequence (mutation(s) are shown in bold
and underlined)(SEQ ID NO: 24):
MGDILAQAVNHAGIDSSSTGPTLTTHSCSVSSAPLNKPTPTSVAVTNTPLPGASATPEL
SPRKKPRKTTRPFKVIIKPPVPPAPIMLPLIKQEDIKPEPDFTIQYRNKIIDTAGCIVISDSE
EEQGEEVETRGATASSPSTGSGTPRVTSPTHPLSQMNHPPLPDPLGRPDEDSSSSSS
SSCSSASDSESESEEMKCSSGGGASVTSSHHGRGGFGGAASSSLLSCGHQSSGGAS
TGPRKKKSKRISELDNEKVRNIMKDKNTPFCTPNVQTRRGRVKIDEVSRMFRNTNRSL
EYKNLPFTIPSMHQVLDEAIKACKTMQVNNKGIQIIYTRNHEVKSEVDAVRCRLGTMCN
LALSTPFLMEATMPVTAPPEVAQRTADACNEGVKAAWSLKELHTHQLCPRSSDYRNM
IIHAATPVDLLGALNLCLPLMQKFPKQVMVRIFSTNQGGFMLPIYETAAKAYAVGQFEQ
PTETPPEDLDTLSLAIEAAIQDLRNKSQ

Having described the invention with reference to the embodiments and illustrative examples, those in the art may appreciate modifications to the invention as described and illustrated that do not depart from the spirit and scope of the invention as disclosed in the specification. The examples are set forth to aid in understanding the invention but are not intended to, and should not be construed to, limit its scope in any way. The examples do not include detailed descriptions of conventional methods. Such methods are well known to those of ordinary skill in the art and are described in numerous publications. All references mentioned herein are incorporated in their entirety.

### Materials and Methods

DATABASE SEARCHING: Tandem mass spectra (MS/MS) were extracted from a gradient 4-20% SDS-PAGE gel (Bio-Rad, USA) via in-gel trypsin digestion and subsequent peptide extraction. Charge state de-convolution and de-isotoping were not performed. All MS/MS samples were analyzed using Sequest (XCorr Only) (Thermo Fisher Scientific, San Jose, CA, USA; version IseNode in Proteome Discoverer 2.1.0.81). Sequest (XCorr Only) was set up to search crap_ncbi.fasta; Heidi_20170828. fasta; human_refseq.fasta (unknown version, 73204 entries) assuming the digestion enzyme non-specific. Sequest (XCorr Only) was searched with a fragment ion mass tolerance of 0.60 Da and a parent ion tolerance of PPM. Carbamidomethyl of cysteine was specified in Sequest (XCorr Only) as a fixed modification. De-amidation of asparagine, oxidation of methionine and acetyl of the N-terminus were specified in Sequest (XCorr Only) as variable modifications.

CRITERIA FOR PROTEIN IDENTIFICATION: Scaffold (version Scaffold_4.8.4, Proteome Software Inc., Portland, OR) was used to validate MS/MS based peptide and protein identifications. Peptide identifications were accepted if they could be established at greater than 36.0% probability by the Scaffold Local FDR algorithm. Protein identifications were accepted if they could be established at greater than 98.0% probability to achieve an FDR less than 1.0% and contained at least 5 identified peptides. Protein probabilities were assigned by the Protein Prophet algorithm [33]. Proteins that contained similar peptides and could not be differentiated based on MS/MS analysis alone were grouped to satisfy the principles of parsimony.

### Example 1. Assessment of the First-Generation Triplex

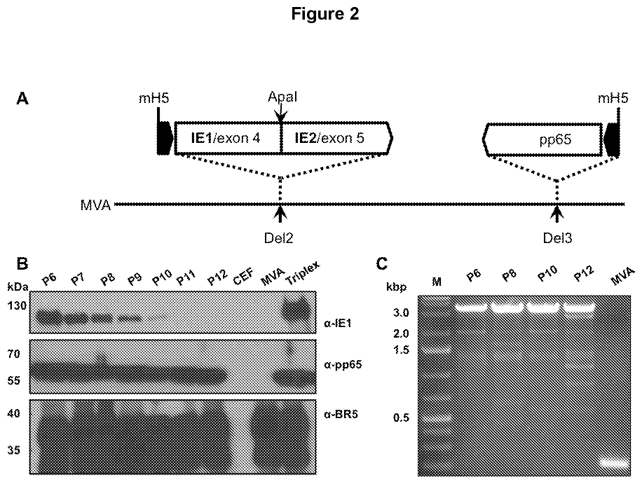

In this study, “stability” was assessed by the integrity of the gene of interest within MVA being monitored via polymerase chain reaction (PCR), DNA sequencing and western blot to ensure the full-length gene is present and full-length protein is present. The original design of Triplex contained a pSyn promoter that, upon serial passaging, caused instability resulting in greatly reduced protein expression. The pSyn promoter was previously replaced with a modified vaccinia virus H5 (mH5) promoter (FIG. 2A), upon which increased IEfusion protein stability was observed while maintaining immunogenicity [1, 2]. To further improve expression stability, the nuclear localization sequence and transcriptional activation domains encoded within exon 2/exon 3 and the overlapping reading frames of IE1 and IE2 from HCMV AD169 were omitted to prevent gene activation events that may be associated with carcinogenesis and reduce the number of possible transcription units. (FIG. 2A) [3]. Therefore, IE1/IE2 fusion with a seamless junction in between, without adding additional nucleotides or amino acids, was inserted into the Del2 site of Modified Vaccinia Ankara (MVA) while unmodified phosphoprotein pp65 [4] was inserted into the Del3 site of MVA [1]. After these modifications, IEfusion stability was observed at the RNA level over 10 viral passages in CEF although not at either the protein (FIG. 2B) or the DNA level (FIG. 2C) [1]. For propagation the IEfusion sample underwent approximately 5 passages prior to evaluation. Thus, in FIG. 2B, the sample marked as P1 was likely passaged five times prior to analysis in CEF. Clinical Triplex used in FIG. 2B, on the other hand, did not undergo as many passages; therefore reduced stability was already observed in P1 for IEfusion (FIGS. 2B and 2C).

Therefore, the first-generation Triplex might not be acceptable to the Food and Drug Administration (FDA) as a Phase 3 manufacturing solution without rigorous validation.

### Example 2. Generation of IEfusion Variants

Although the genomic architecture of the first-generation Triplex and the new Triplex constructs disclosed herein are similar, IEfusion inserted at other sites in MVA (Scheme I shown in FIG. 1) was evaluated in addition to exploring gene modifications to IEfusion to reduce spontaneous mutation hot spots and/or increase expression via codon optimization for pox virus (i.e., wobble position (4 nt) and vaccinia virus expression (VacO) optimization). First generation Triplex contains IEfusion in Del2 and pp65 in Del3 in MVA using the HCMV AD169 DNA sequence, generated via a transfer plasmid that would facilitate homologous recombination of IEfusion into wild-type MVA [3]. See FIG. 2. Because this process can be time consuming, the Bacterial Artificial Chromosome (BAC) technology was utilized. By applying BAC technology [11, 12] and en passant mutagenesis [13, 14] to generate new MVA constructs expressing pp65 and various iterations of IEfusion, each of the proposed viral constructs (Table 2) was rapidly generated and tested.

TABLE 2
MVA Constructs Expressing IEfusion
Generated via BAC Technology

| | Insertion Sites | | | | | |
|---|---|---|---|---|---|---|
| Gene | Del2 | G1L/I8R | IGR3 | 044/045L | Del3 | New Del3 |
| IEfus | X | X | X | ND | ND | ND |
| IEfus 4 nt | X | X | X | X | ND | ND |
| IEfus VacO | X | X | X | ND | ND | ND |
| IE1 | ND | ✓ | X | X | ND | ND |
| IE1 4 nt | ND | ✓ | ✓ | X | ND | ND |
| IE1 VacO | ND | ✓ | ✓ | X | ND | ND |
| IE2 | ND | X | X | X | X | X |
| IE2 4 nt | ND | X | X | X | X | X |
| IE2 VacO | ND | X | X | X | X | X |
| pp65 | ND | ND | ND | ND | ✓ | ND |

“X” denotes unstable; “✓” denotes stable site; and “ND” indicates not determined.

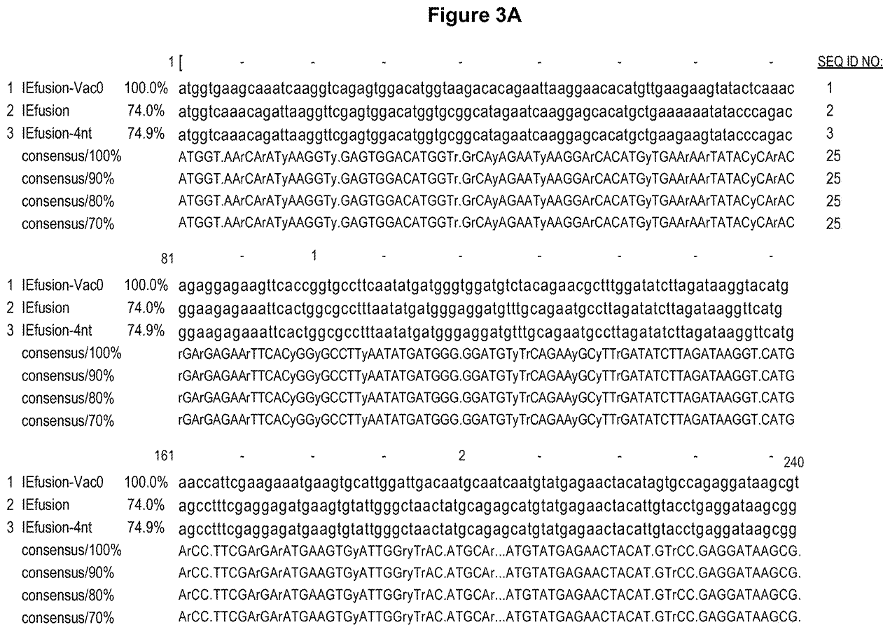

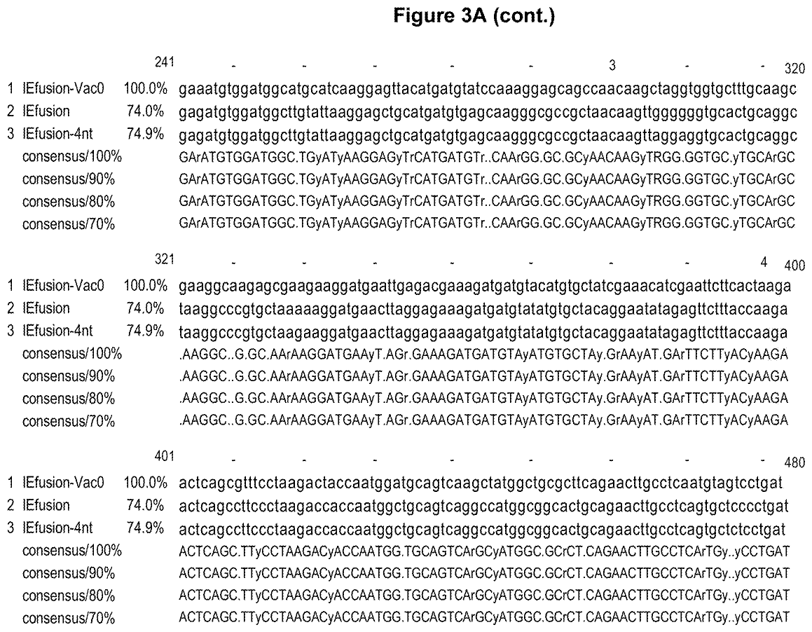

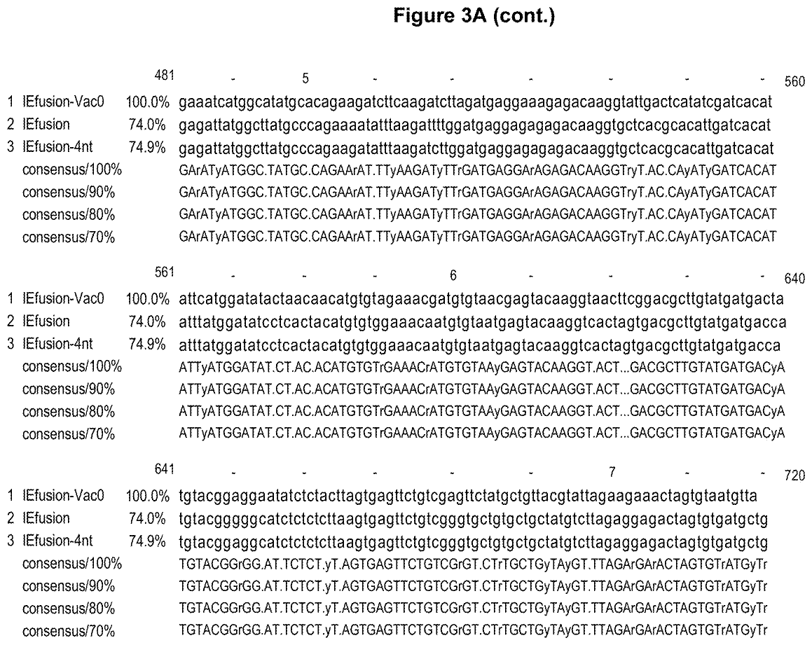

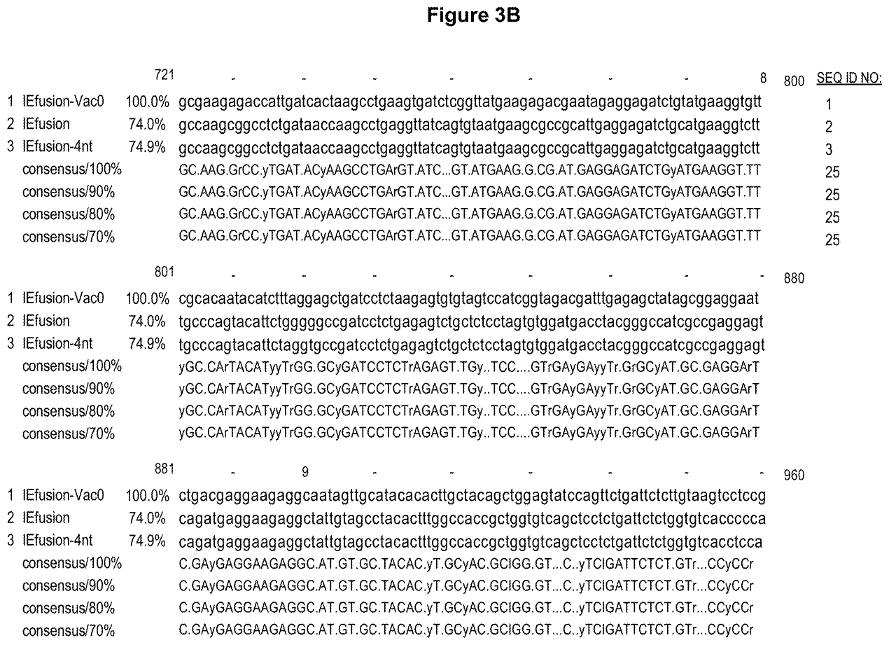

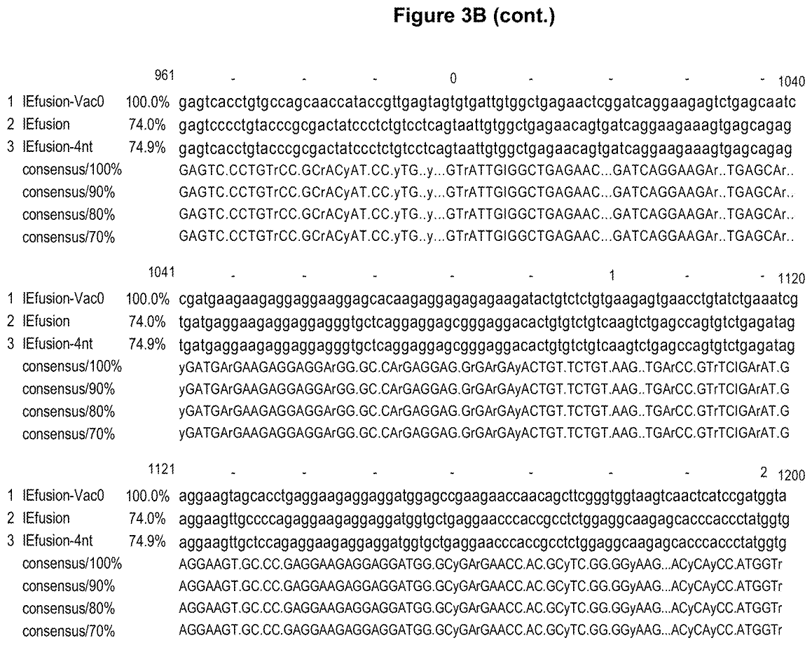

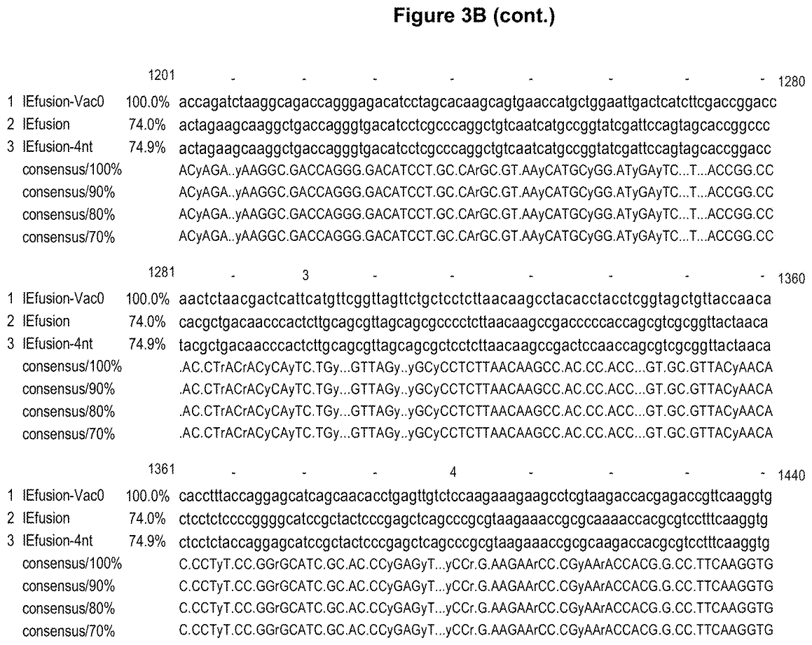

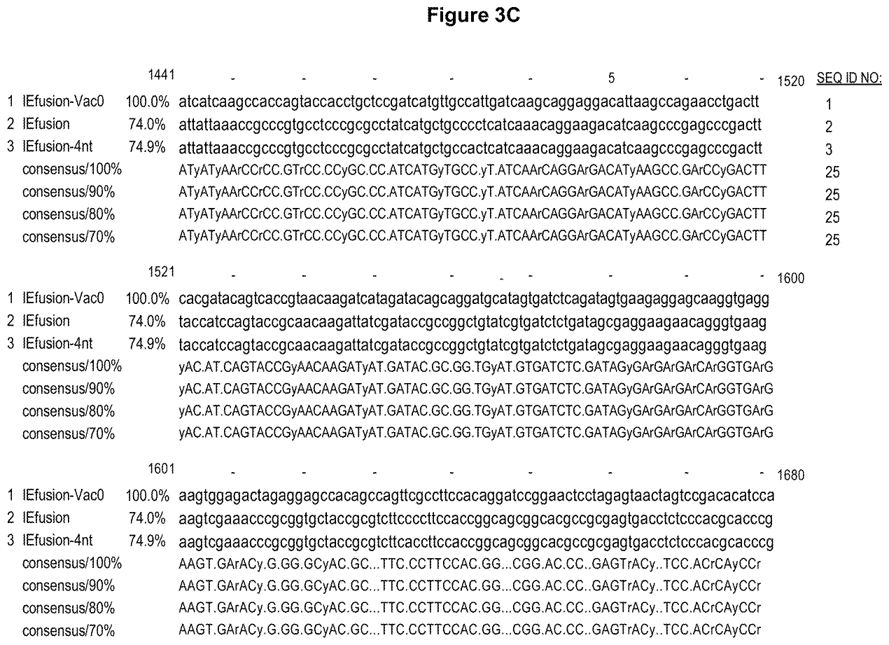

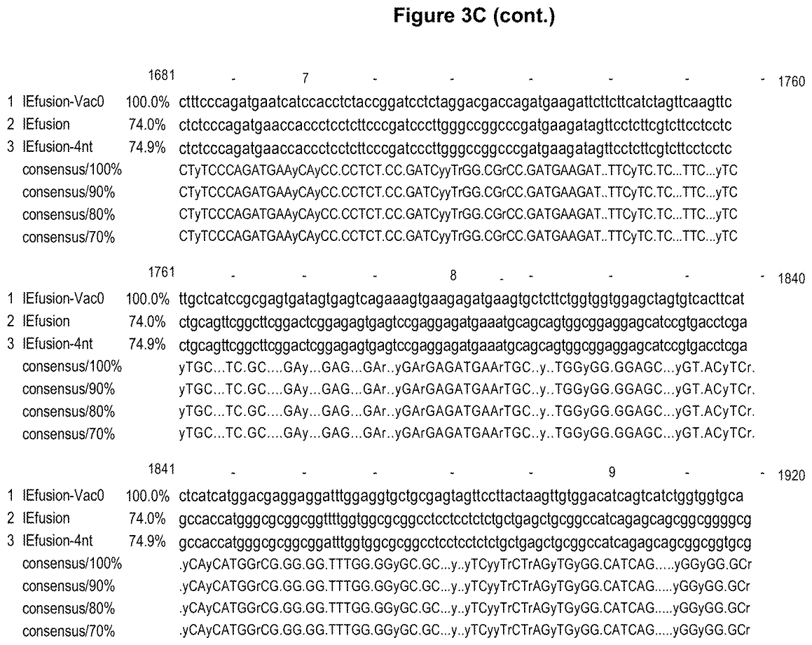

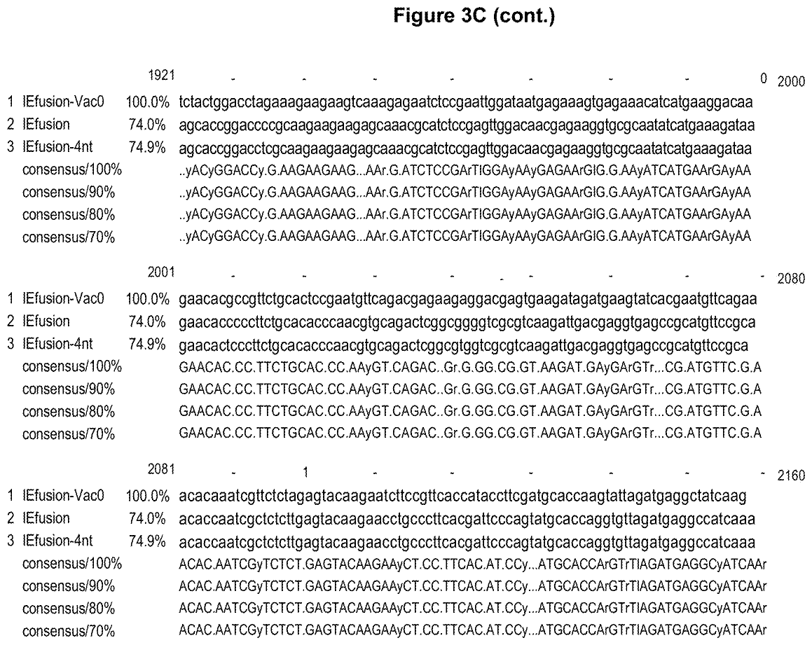

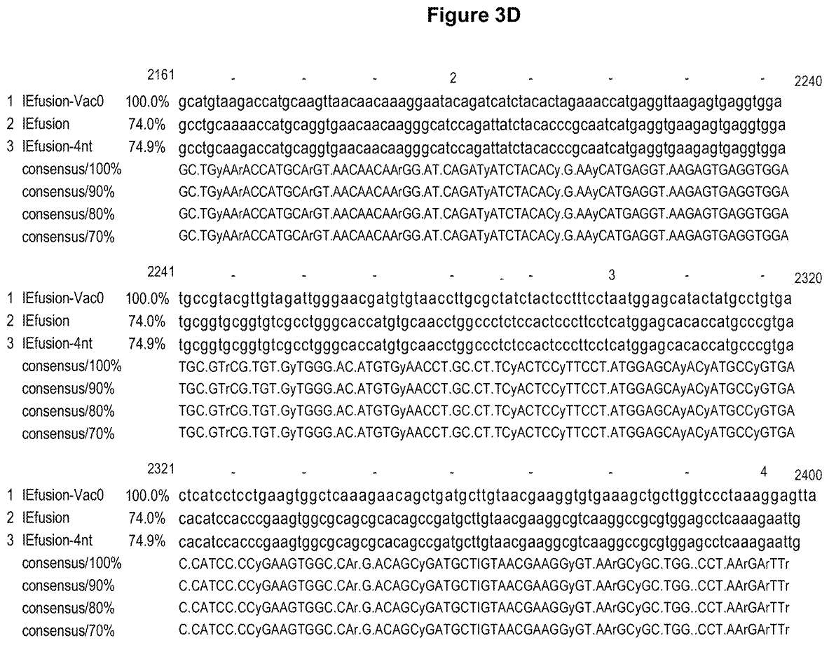

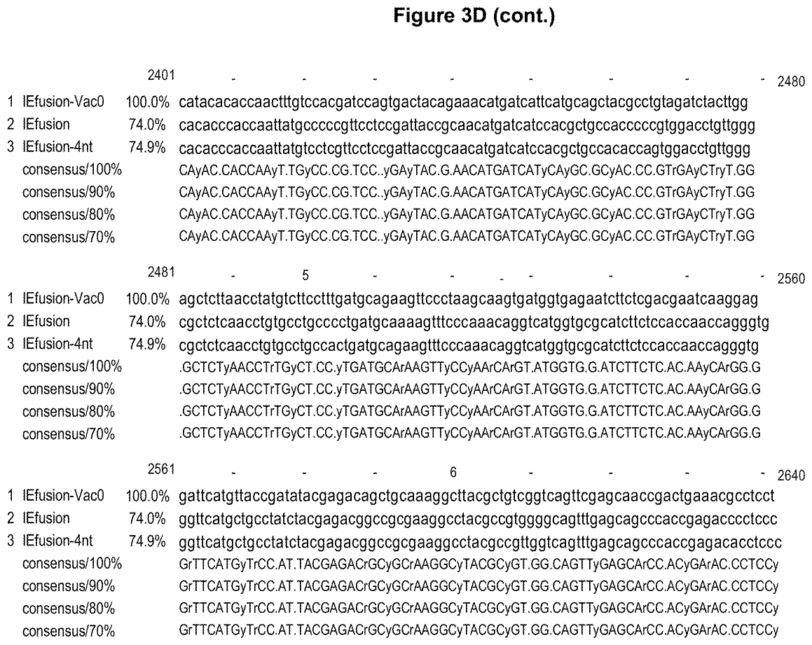

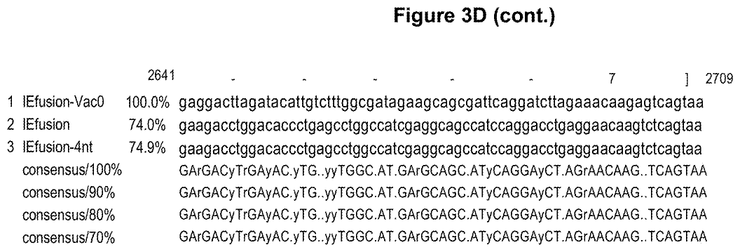

As shown in Table 2, some versions of IEfusion (IEfus) were inserted into the following sites in MVA: Del2, G1L, IGR3, or 044L/045L. After inserting IEfusion into Del3, G1L/18R, IGR3, and 044L/045L, some sites did aid in stabilizing gene expression to some extent (data not shown), but likely insufficient to meet FDA standards for late stage clinical evaluation. FIG. 3 shows the nucleic acid sequence alignment of IEfusion constructs. FIG. 4 shows that IEfusion 4nt stabilized IEfusion expression beyond five viral passages (P5) when constructed within the IGR3 site in MVA.

### Example 3. Assessment of IEfusion Variants

Mutation hot spots were removed by disrupting the runs of consecutive C or G nucleotide bases [15], followed by vaccinia virus codon (designated as VacO) optimizing the DNA sequence of IEfusion. Constructs shown in Table 2 marked with an “X” were analyzed for stability via PCR and by western blot to monitor the integrity of the gene within its insertion site and expression after passage. This type of analysis provides insight regarding instability at either the DNA or protein levels.

Based on the knowledge that removal of spontaneous mutation hot spots minimizes instability, all IEfusion constructs shown in Table 2 were evaluated.

IEfusion 4nt inserted in IGR3 (FIG. 4) shows increased stability over various IEfusion constructs that were modified and inserted into Del2, G1L, and 044L/045L insertion sites (Table 2 and FIG. 4A). The genes of interest were amplified via PCR for gene stability analyses, including the flanking regions in the MVA (expected IEfusion size shown in Table 3).

TABLE 3
PCR product sizes for genes engineered within specific sites

| | G1L/I8R | IGR3 | 044/045L | Del3 | Protein Size |
|---|---|---|---|---|---|
| Gene | (bp) | (bp) | (bp) | (bp) | (kDa) |
| IEfusion | 4274 | 3640 | n/a | n/a | ~130 |
| IE1 | 2783 | 2155 | 1998 | n/a | ~55 |
| IE2 | 3051 | 2423 | 2265 | n/a | ~68 |
| pp65 | n/a | n/a | n/a | 2923 | ~65 |
| Empty | 1538 | 818 | 667 | 1072 | n/a |

When the integrity of the full-length gene was compromised during passaging, non-specific PCR products would emerge and aberrant DNA sequencing results were observed around passage 3 (P3) or products similar to the size of the negative control (FIG. 4B); however, with instability at the protein level, a decrease in expression of the full-length product was observed at P7 (FIG. 4C). Although it seemed that some improvement in stability was observed over serial viral passaging in CEF (data not shown), protein expression of full-length IEfusion was not maintained over prolonged passaging. Based on this information, it was concluded that IE1 and IE2 would not be stably expressed as a protein fusion of ˜130 kDa unless modifications were made at either the DNA or amino acid level. Consequently, IEfusion without further modification as a fused sequence may not be optimal for maintaining stable expression of IEfusion in MVA that withstands ten or more passages.

### Example 4: Development of an Innovative Strategy for Stable Expression of IE1, IE2, and pp65 in MVA

The goal was to find the most stable combination of insertion sites and gene modifications to generate an MVA that stably expresses all three antigens at a minimum of 10 passages for large-scale propagation of the vaccine virus. A new vaccine construction strategy was initiated considering three main points to find the most stable combination of insertion sites that allows stable expression of IE1, IE2, and pp65: 1) splitting IEfusion into its IE1/IE2 components; 2) inserting variant gene sequences of all three genes into separate insertion sites in MVA; and 3) explore new insertion sites in MVA. Since there was limited success in enhancing stability with IE1 and IE2 as a fusion protein post nucleotide modifications, IE1 and IE2 were separated and each gene was inserted in separate insertion sites, and then stability of each gene in each of the new sites was analyzed. The original IEfusion construct was used as a template to split IE1 and IE2, with each component under the control of separate mH5 promoters [1]. Table 2 shows all the constructs generated in an attempt to give rise to an MVA that stably expresses IE1 and IE2 over passages. Furthermore, other modifications, such as removal of consecutive C and G nucleotides as well as codon-optimization of the genes as was done for IEfusion, were incorporated. Different sequence modifications of IE1 and IE2 genes were analyzed for stability in CEFs, also using BAC technology to generate the various MVA constructs. Because instability in Del2 has been observed [15], Del2 site was not further pursued as a candidate site.

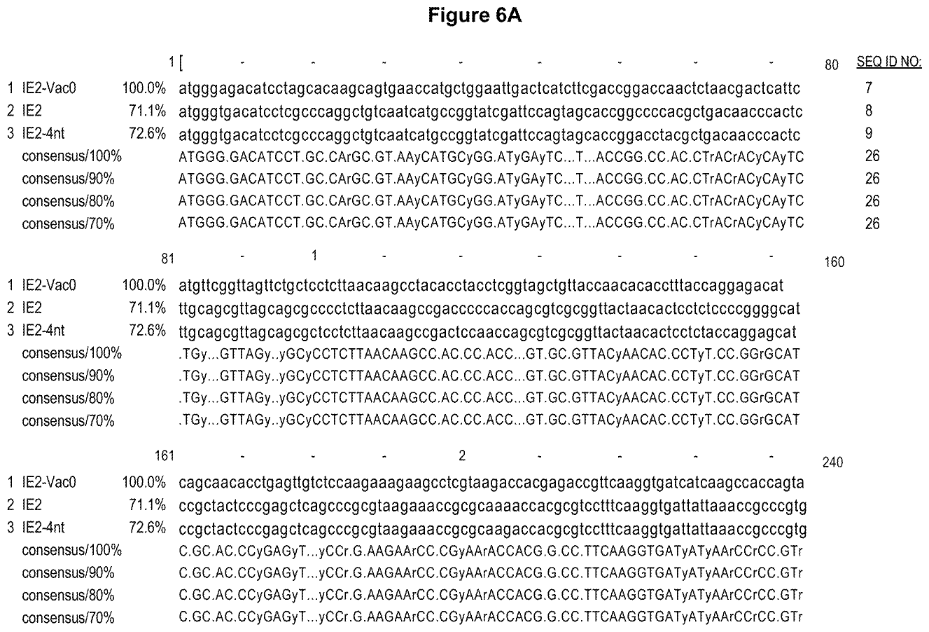

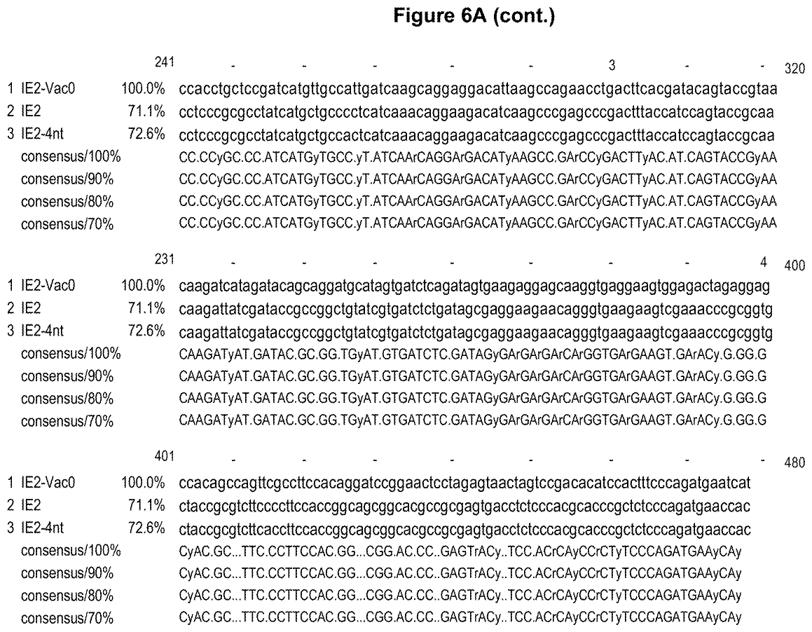

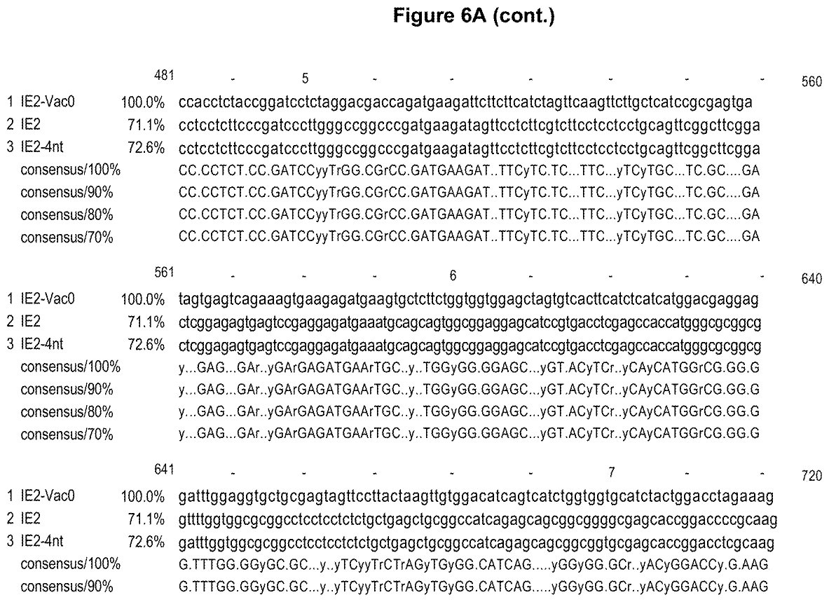

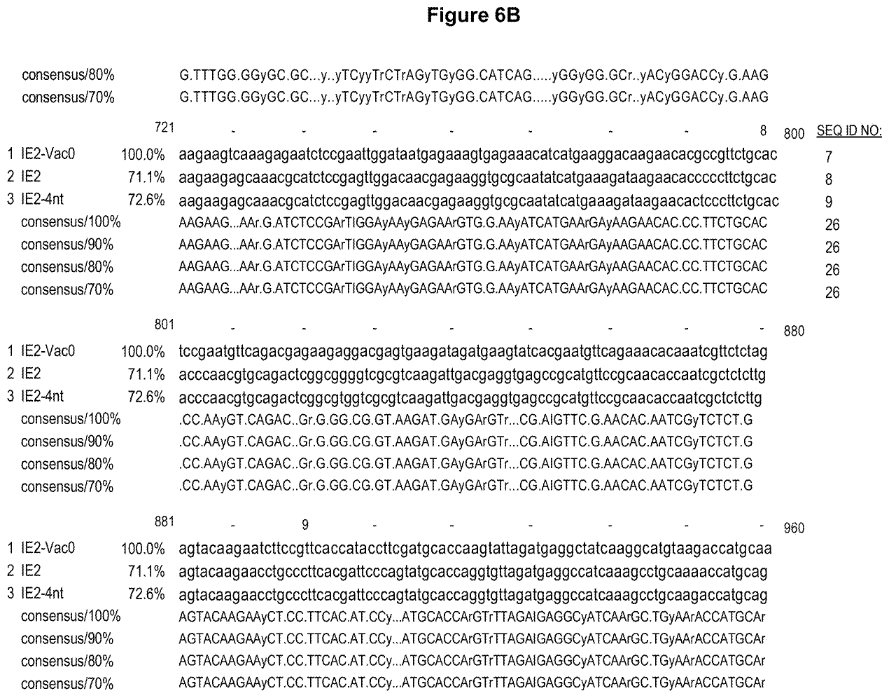

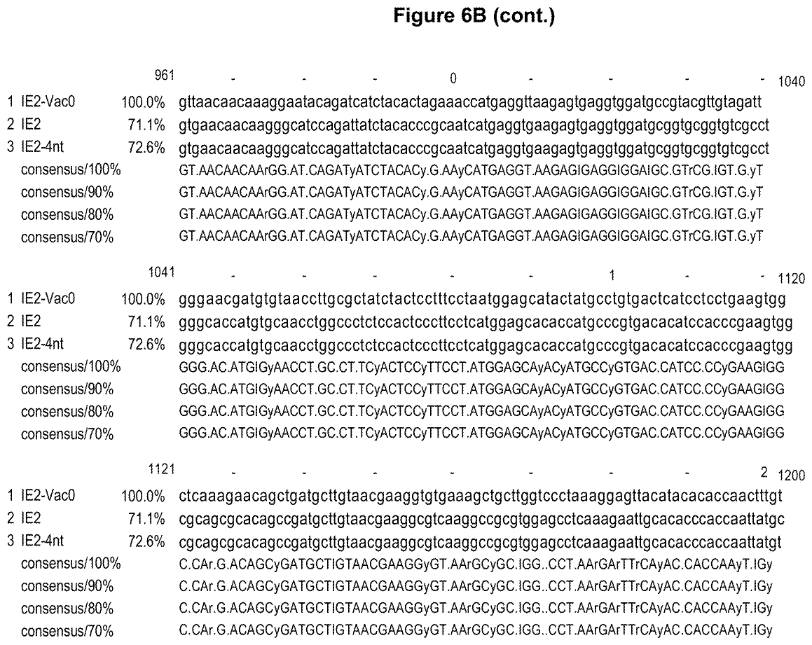

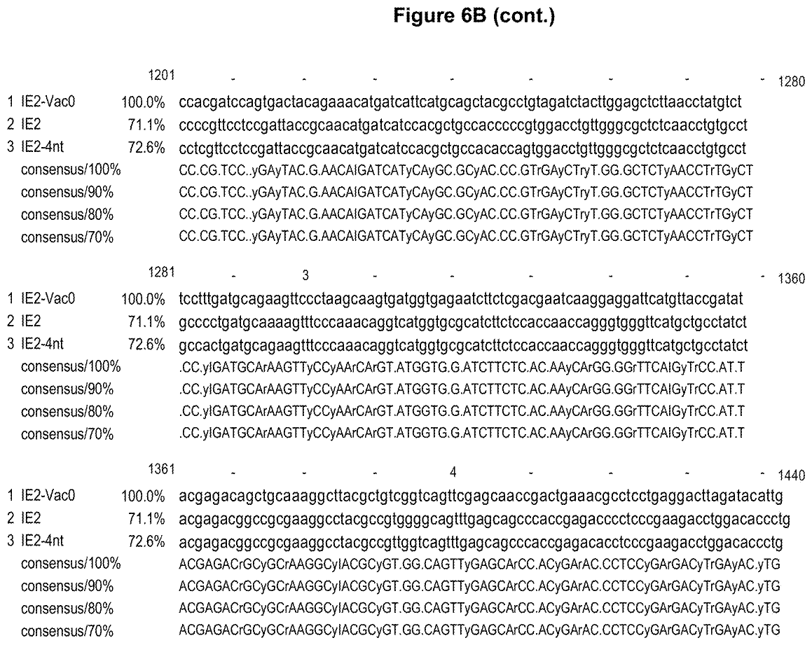

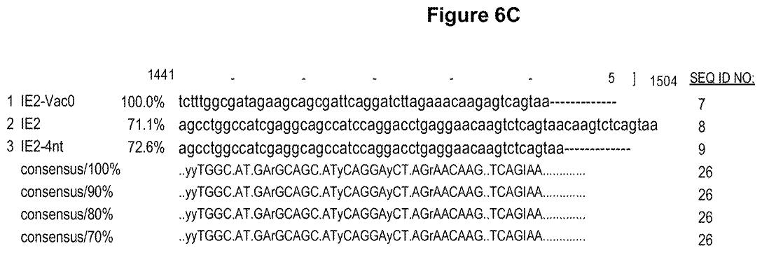

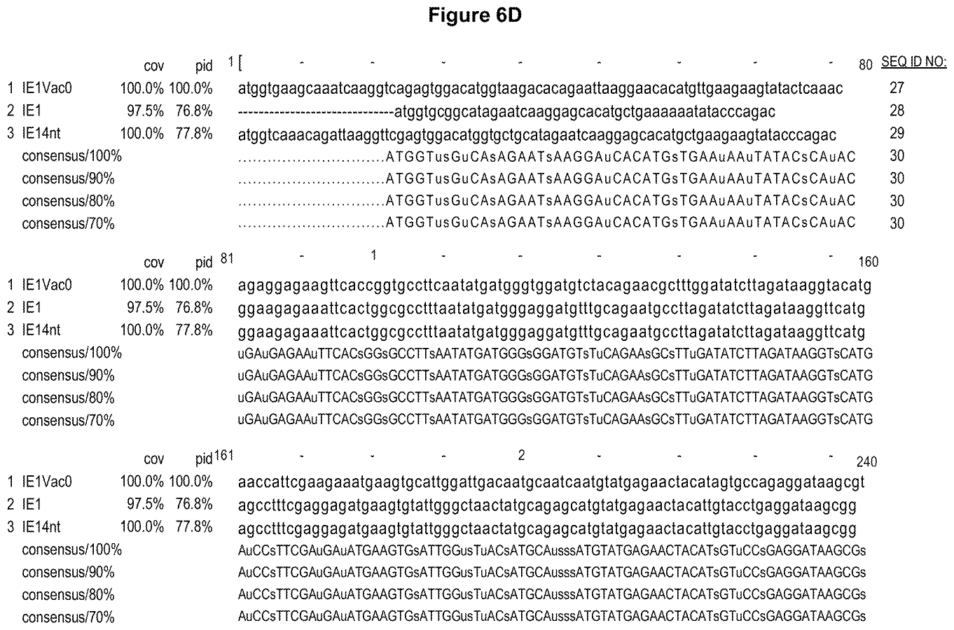

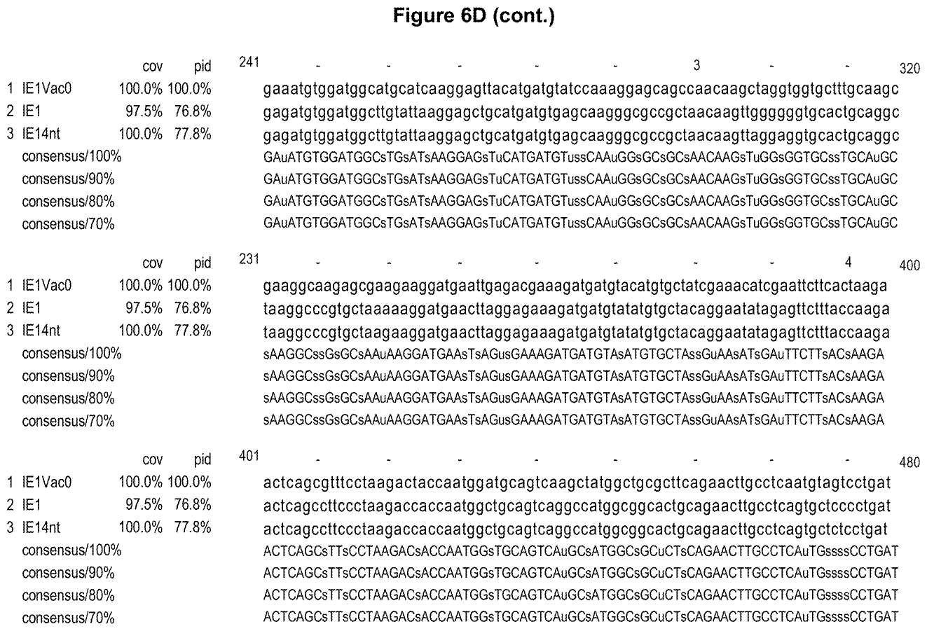

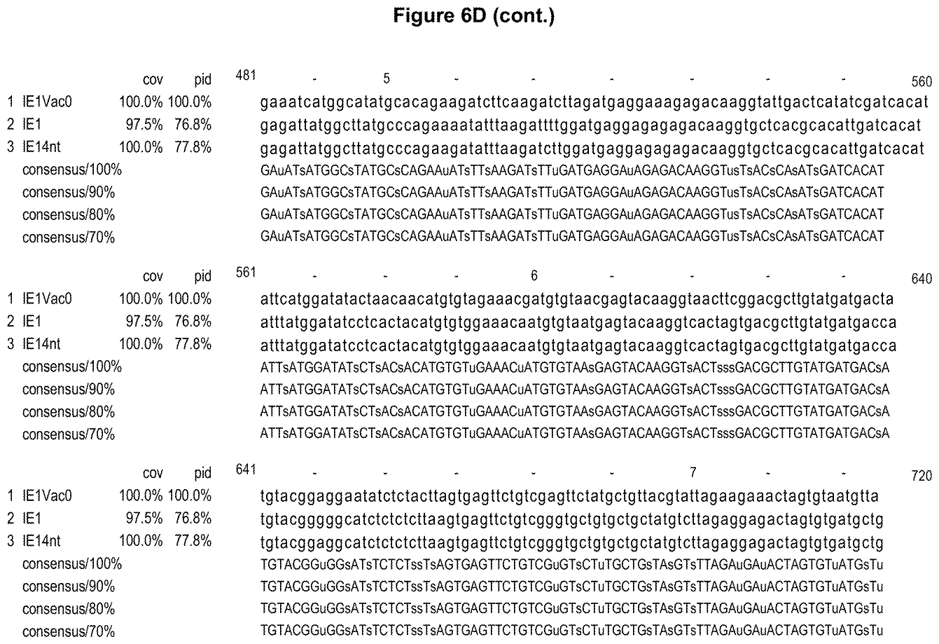

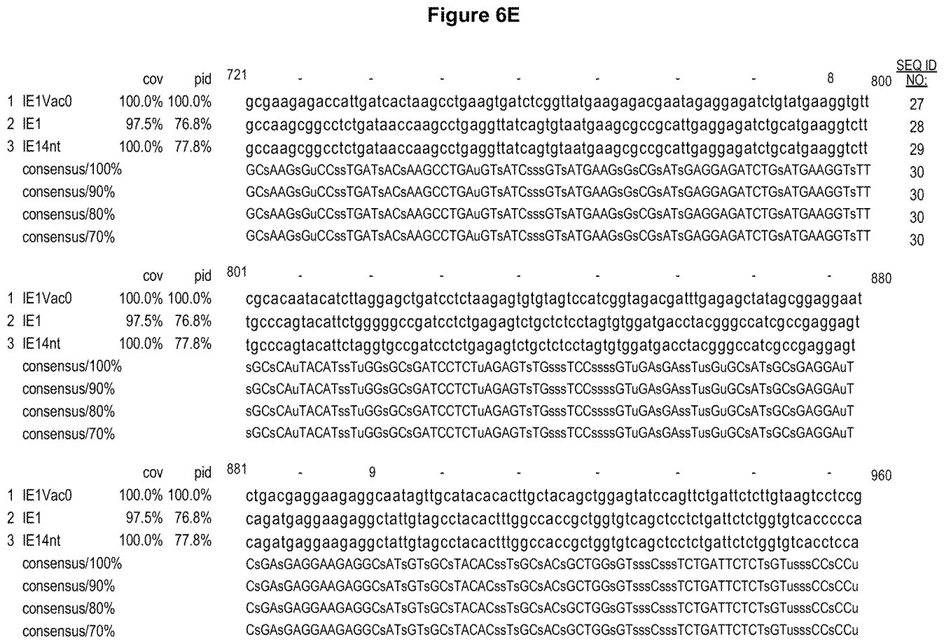

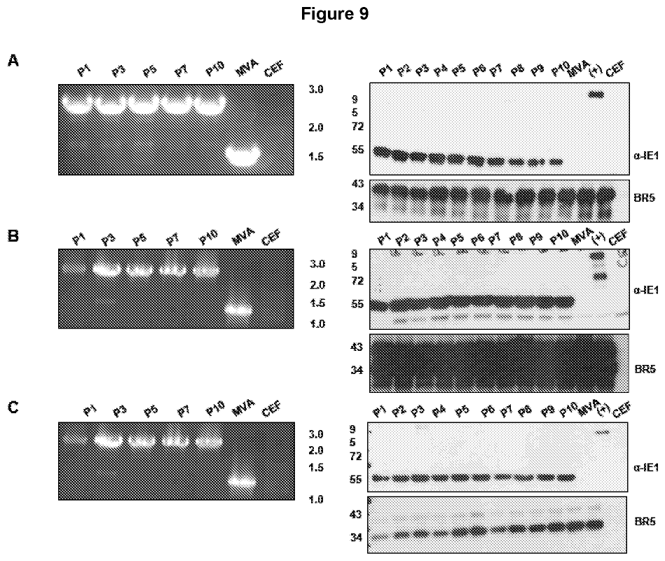

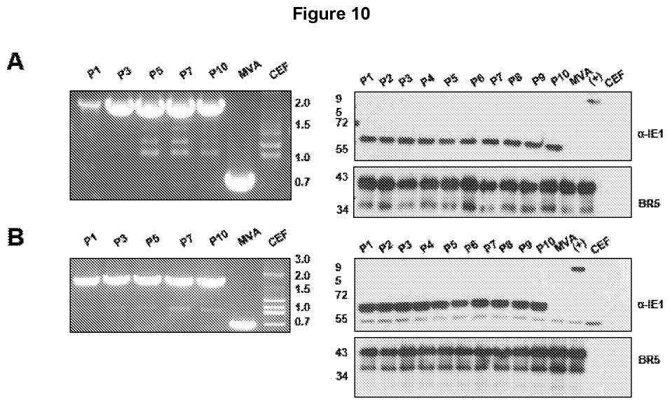

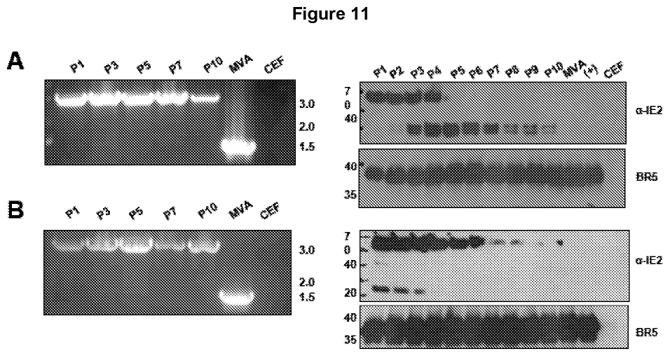

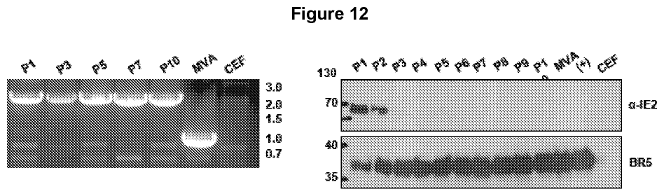

Based on the experimental setup to evaluate stability of the genes in CEFs, five potential candidates for the IE1 gene to be expressed in either G1L or IGR3 were obtained (Table 2). Only IE1 (non-codon optimized (NCO), four nucleotide optimization (4nt), and vaccinia optimized (VacO)) showed stability in G1L (FIG. 9) while only IE1 (4nt) and IE1 (VacO) demonstrated stability in IGR3 (FIG. 10). IE2 constructs were more difficult to generate upon virus reconstitution in BHK cells using fowl pox helper virus [11, 16]. Attempting to reconstitute IE2-containing MVA was more difficult than what was experienced with IE1. After many transfection attempts, followed by stability analyses, stable IE2-expressing MVA constructs that produced full-length IE2 protein over several virus passages in CEFs were not obtained (FIGS. 11 and 12). FIG. 6 shows the nucleic acid sequence alignment of IE2 constructs.

### Example 5: Exploration of IE2 DNA and Amino Acid Sequences

Although none of the IE2-expressing MVA constructs produced full-length IE2 protein, one construct, MVA::IE2 (044L/045L) (Table 2), expressed IE2-related products throughout all ten virus passages (FIG. 7). Alternative protein products of IE2, resulting in ˜20 kDa and ˜40 kDa fragments, have been previously described for IE2 [17-19]. To determine the identity of these alternative protein products, an in-gel trypsin digestion, followed by LC-MS/MS, was performed. Mass spectrometry data analysis revealed that the ˜20 kDa product was mostly the C-terminal portion (48% coverage) of IE2 whereas the ˜40 kDa product was mostly the N-terminus (34% coverage) (Table 4). Full-length IE2 was included in analyses as control (protein probability calculations performed as described in materials and methods).

TABLE 4
Mass spectrometry identification of peptides
corresponding to IE2 from HCMV AD169

| | Exclusive | Exclusive | % | Max | Proba- |
|---|---|---|---|---|---|
| kDa | Unique Spectra | Unique Peptides | Coverage | X_{corr} | bility |
| 63 | 160 | 94 | 83 | 3.37 | 100% |
| 40 | 31 | 22 | 34 | 3.94 | 100% |
| 20 | 37 | 27 | 48 | 3.19 | 100% |

As observed in FIG. 7B, there was a concomitant disappearance of both ˜20 kDa and the full-length ˜60 kDa product, while the ˜40 kDa product remained “stable” over ten passages. These results suggest that perhaps there is an element within the ˜20 kDa product that may not be tolerated by either CEF cells or MVA. Upon DNA sequencing of PCR products in an attempt to identify mutations within regions of the DNA sequence, the IE2 gene sequence was observed to be stochastically mutated during passaging, thereby resulting in premature stop codons within IE2, yielding a truncated, yet stable, IE2 product that is recognized by an IE2-specific antibody [17].

### Example 6: Generation of IE2 Mutations of C-Terminal Histidines

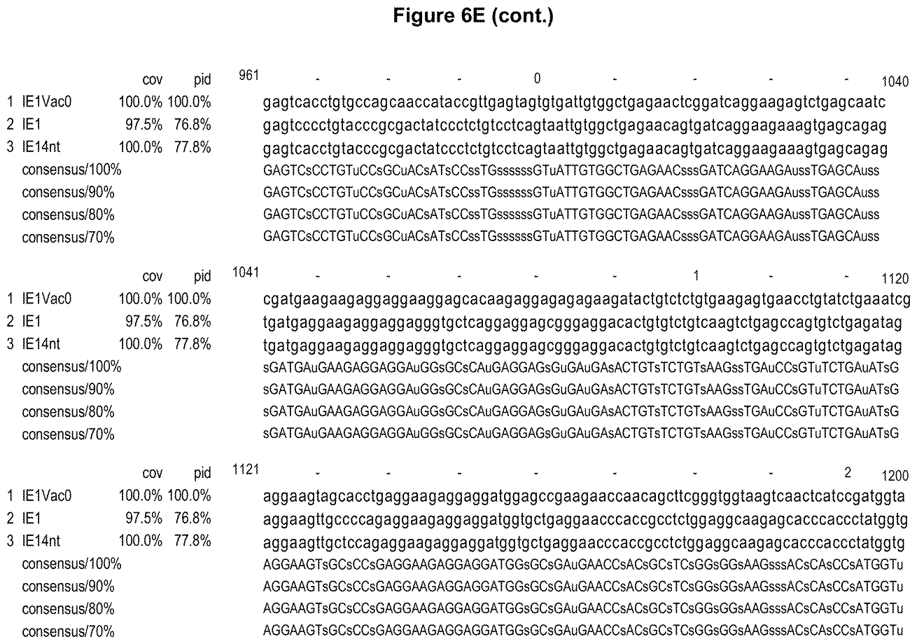

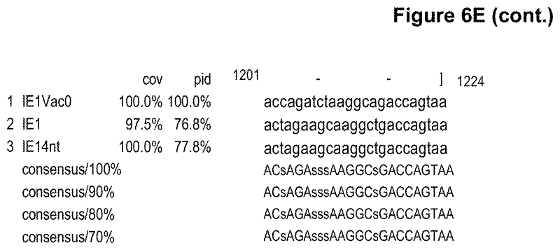

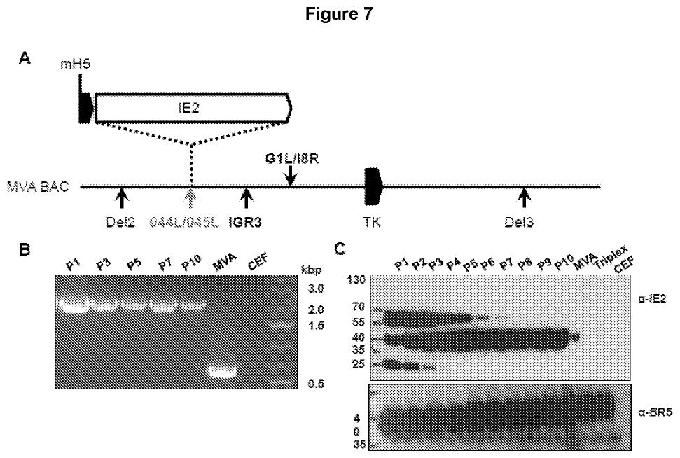

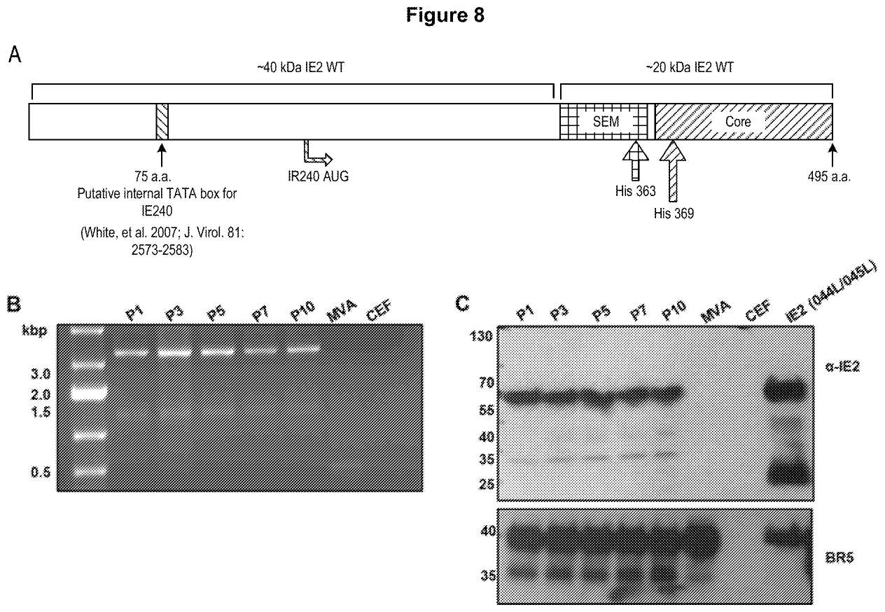

IE1 has properties at the nucleotide level that render it unstable in some locations; inserting IE1 into a different site in MVA mitigated instability. IE2 instability could not be resolved solely by this method. Putative IE2 functional domains have been reported [20]. The C-terminus of IE2 has been described as part of a “core” domain, important for DNA binding, transactivation, and autorepression (FIG. 8A). The region adjacent to the core domain contains a Zn-finger binding domain that can be mutated without affecting IE2 protein interaction with DNA. The area adjacent to the core domain was named the “specific and essential modulator” domain (SEM) (FIG. 8A). Although mutations within the SEM region did not impair all functions previously associated with IE2, it has been observed that different sequence requirements within this region affect different IE2 functions. Two His residues within the C-terminus of IE2 were identified within the presumed Zn-finger binding domain (FIG. 8A), His446 and His452 [21, 22]. Mutating these IE2 residues does not abolish DNA binding capabilities nor hinder IE2 expression within HCMV or in a heterologous system such as adenovirus [23]. As a result of mutating the last 37 amino acid residues, it was determined to be required for IE2 autorepressive and transactivating functions [24-27]. Two histidine (H) residues were mutated to alanine (A) using site-directed mutagenesis, although no other C-terminal residues from either the SEM nor core domains were altered. This determination was due to the notion that if the last ˜37 amino acid residues in the C-terminus are important for IE2 activities, these residues may possibly encode immunogenic epitopes. Mutating residues within the SEM are well tolerated, without affecting IE2 activity [20]; therefore, whether modifications within this region could help with the genetic and protein stability of the IE2 gene and its protein product in MVA upon viral passaging in CEFs was further explored.

There were challenges to find a location and identify a sequence for IE2 that would render it “stable” for expression in MVA. Similar to the original construction of Triplex containing IEfusion, N-terminal signal peptide, nuclear localization sequences, as well as activation domains (exons 1, 2, and 3; amino acids 1-85) were omitted when generating MVA expressing IE2 [18, 26]; hence, these deletions changed the amino acid residue numbers from His446 and His452 to His363 to His369, respectively. The following single and double mutants of IE2 were generated for insertion into the 044/045L site on MVA: H363A, H369A, and H363A/H369A (Table 5). In Table 5 below, ×□=unstable; ✓□=stable. Roman numeral identifies the iteration and mutation of IE2.

TABLE 5
Summary of IE2 mutants within MVA.

Mutants

| Version | H363A | H369A | H363/369A |
|---|---|---|---|
| IE2 NCO | ✓□(i) | □✓(ii) | □✓(iii) |
| IE2 4nt | □✓(iv) | □✓(v) | □✓(vi) |
| IE2 VacO | X□ | □✓(vii) | □✓(viii) |

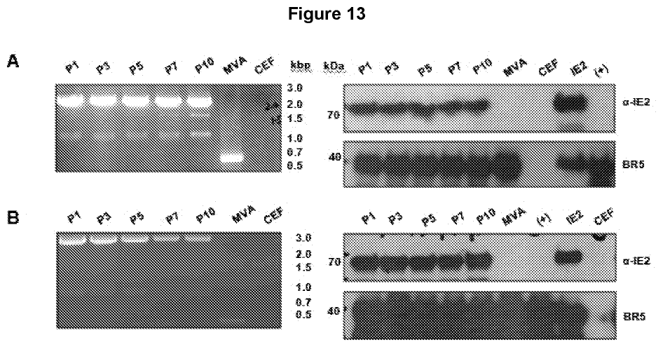

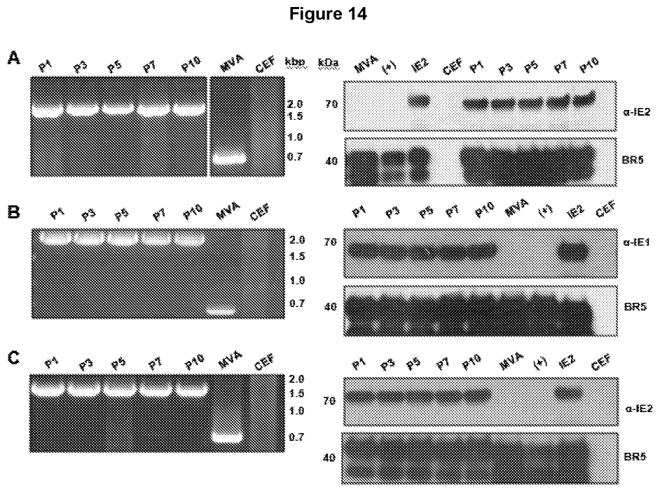

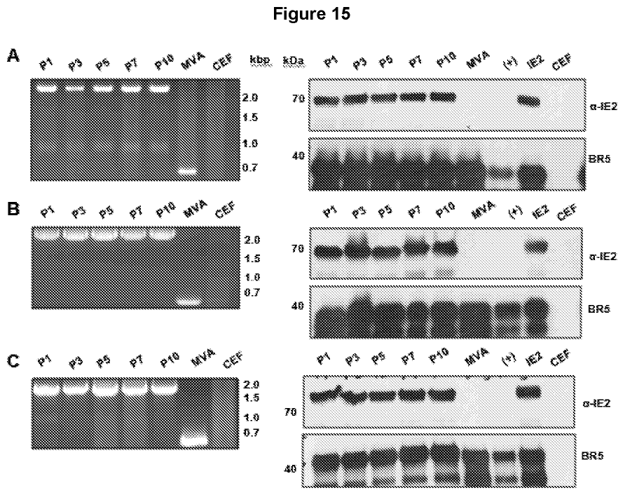

After transfection and viral reconstitution, all constructs were passaged in CEFs. It was observed that upon serial passaging, IE2 expression was stable based on Western blot analysis (FIG. 8C). Furthermore, PCR analysis revealed amplification of the expected gene product resulting in a “stable” construct upon 10 passages (FIGS. 8B, 13-15). While only IE2 NCO and IE2 4nt iterations could be generated for the H363A mutant (FIG. 13), IE2 NCO, 4nt, and VacO versions of H369A (FIG. 14) and H363A/H369A (FIG. 15) were constructed. Eight versions of IE2 mutants were passaged and analyzed via PCR and Western Blot analysis. These results suggest that mutating His residues within the C-terminus containing the putative Zn-binding domain helps stabilize IE2 expression and gene sequence upon viral passage.

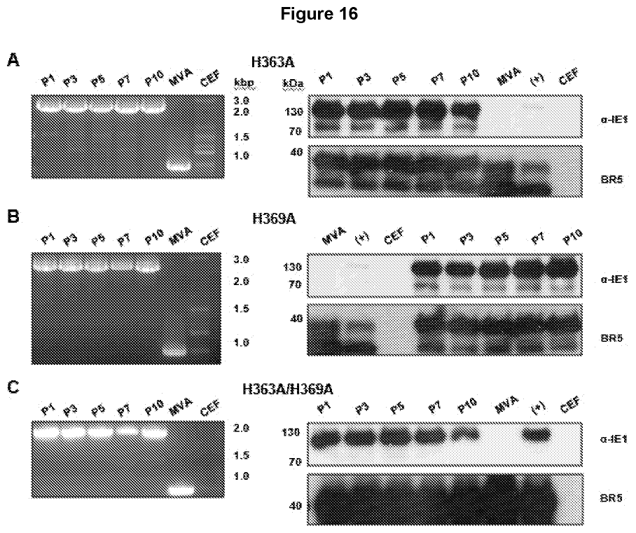

Identifying IE2 as a major contributor of instability, IEfusion was reassessed. The corresponding His was mutated to Ala residues on the C-terminus of IEfusion. Based on previous data (FIG. 4), residues on the 4nt version of IEfusion that was engineered for insertion into the IGR3 site were mutated—either H363A and/or H369A mutants of IEfusion 4nt. In contrast to Triplex, prolonged stability to P10 was observed for all three mutant versions of IEfusion 4nt at the protein level; however minor non-specific PCR products were observed for the double mutant (FIG. 16C, left). These constructs became candidates for further analysis in combination with pp65.

### Example 7: Combining all Three HCMV Antigens into a Single MVA

Two constructs were identified to stably express both IE1 and pp65: (A) MVA BAC::IE1 4nt (IGR3)::pp65 (Del3) and (B) MVA BAC::IE1 VacO (IGR3)::pp65 (Del3). Once the effect of mutating H363A and/or H369A on IE2 stability was evaluated, various mutant IE2 versions were inserted into MVA site 044L/045L of either of the aforementioned constructs. Based on the previous studies evaluating the stability of IE2, it became apparent that, although IE1 has properties at the nucleotide level that render it unstable in some locations, the instability was mitigated by inserting IE1 into a different site in MVA. In contrast, IE2 was difficult to find a location and sequence that would render it “stable” for expression in MVA. Upon mutation of C-terminal His, the gene and protein stability within the 044L/045L site was improved. Identifying IE2 as a major contributor of instability, IEfusion was reassessed. Mutants of IEfusion were generated, including mutagenizing the His residues that lie on the C-terminus of the IE2 portion. However, due to the instability of genes inserted in Del2, inserting genes within that site was not pursued. Either H363A and/or H369A mutants of IEfusion, IEfusion 4nt, and IEfusion VacO were generated. These variants of IEfusion were either inserted in IGR3 or 044L/045L, while also containing pp65 in Del3. Upon completion of the Triplex variants, vaccination of transgenic HLA-expressing mice can be used to compare immunogenicity generated by IEfusion mutants versus re-derived Triplex with separated IE1 and IE2 genes, all with His mutations as described in FIG. 8.

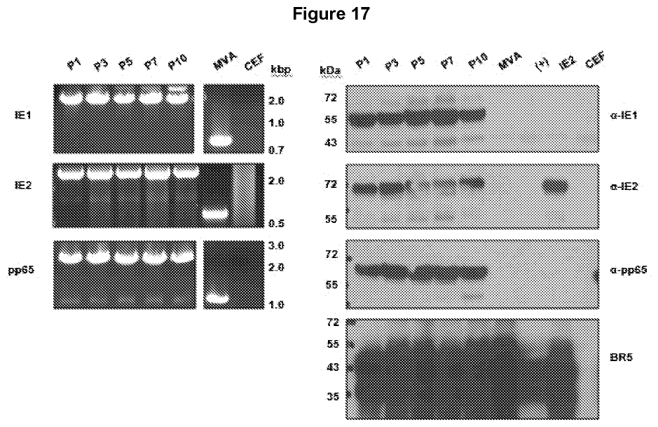

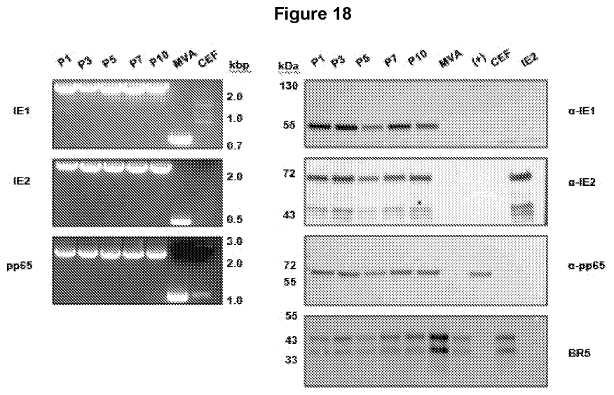

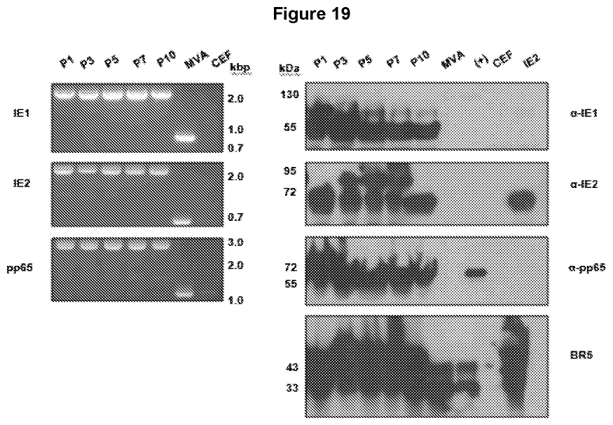

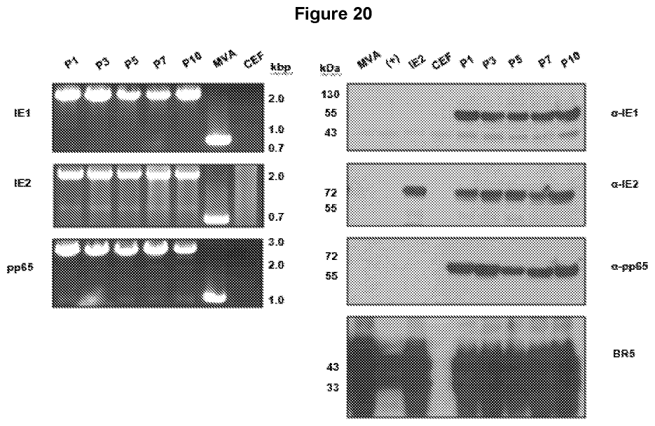

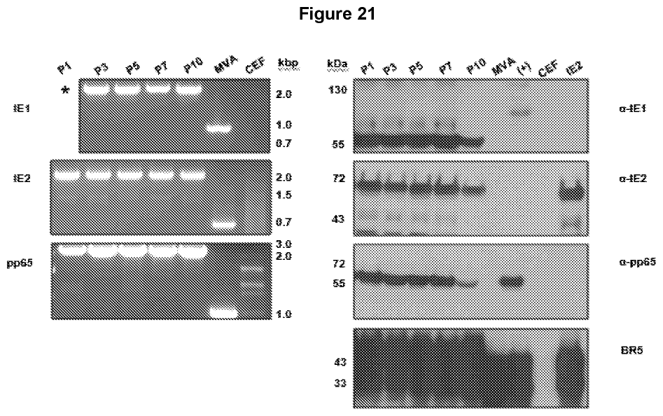

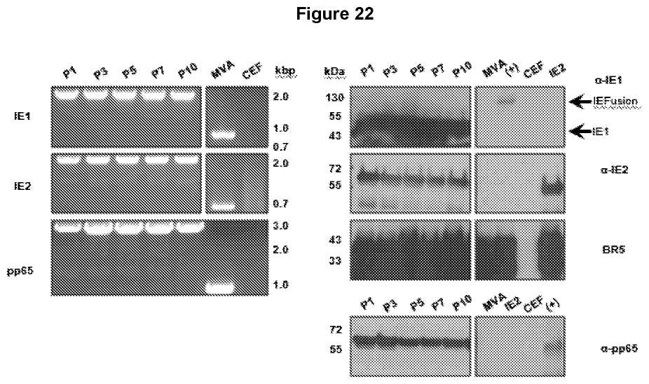

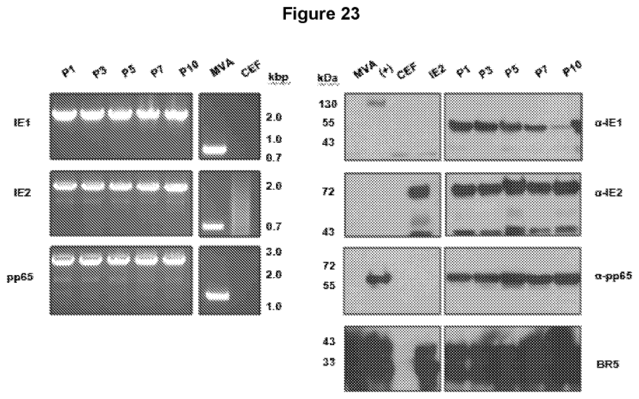

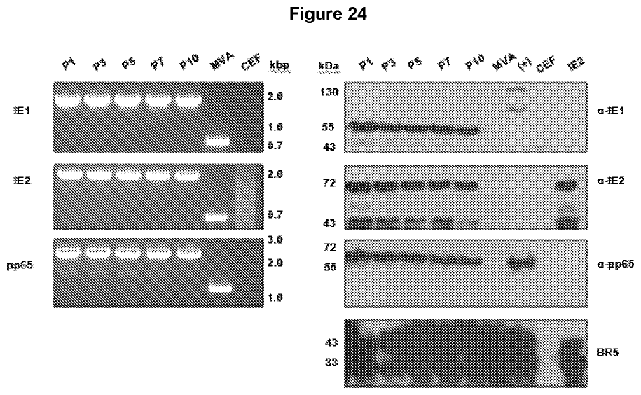

Furthermore, mutation of C-terminal His prolonged gene and protein stability within the 044L/045L site for IE2 and IGR3 site for IEfusion. Three constructs (IE2 NCO H363A (i); IE2 4nt H369A (v); IE2 4nt H363A/H369A (vi)) in the context of (A) appeared stable up to P10 with all three antigens (IE1, IE2, pp65) being expressed from a single MVA (FIGS. 17-19). Five constructs in the context of (B) appeared to stably express all three antigens from a single MVA (FIGS. 20-24). However, construct B(ii) (FIG. 21) appeared to show decreased pp65 expression by P10. A decrease of IE1 expression in construct B(v) was also observed by P10 (FIG. 23). Overall, it appeared that six constructs (A(i), A(v), A(vi), B(i), B(iii), and B(vii)) showed stable expression of all three antigens over ten viral serial passages, as assessed by PCR and Western Blot analyses. These results show that single and double mutations within the putative Zn-finger binding domain of IE2 helped stabilize expression of all three antigens.

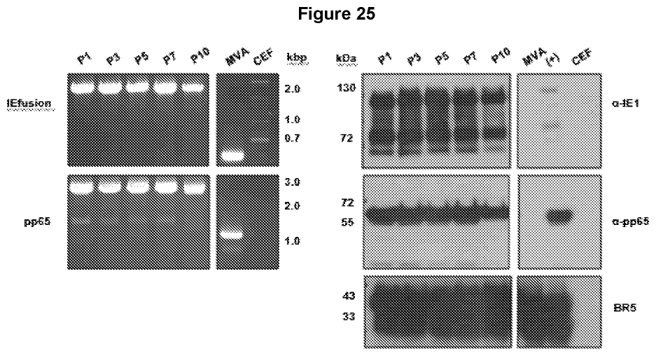

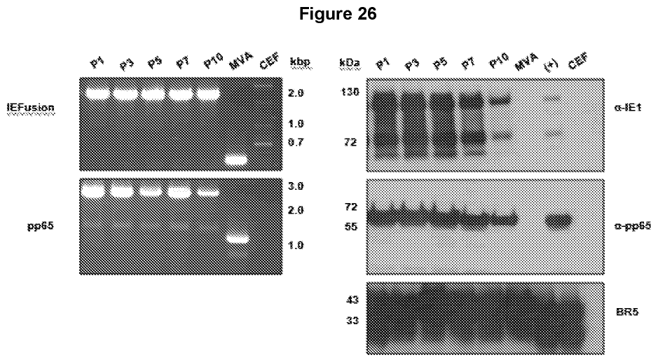

Mutants of IEfusion were also characterized for stable expression over ten serial virus passages (FIGS. 25 and 26). IEfusion 4nt H363A (IGR3)::pp65 (Del3) showed increased stability (FIG. 25) beyond P7 compared to IEfusion IGR3 (FIG. 9). A slight decrease of IEfusion PCR product was observed for P10 (FIG. 25, left) while a slight decrease in pp65 protein was observed via Western blot (FIG. 25, right) by P10. In contrast IEfusion 4nt H369A (IGR3)::pp65 (Del3) demonstrated a decrease in IEfusion and pp65 proteins (FIG. 26, right) by P7<n>P10. No significant decrease observed for IEfusion PCR product; however, there was a marked decrease of pp65 PCR product by P10. These results show that IEfusion 4nt H363A (IGR3)::pp65 (Del3) stably expresses these CMV antigens and the H363A mutation aids in the maintenance of intact protein expression and PCR product integrity.

### Example 8: Immunogenicity of the Second-Generation Triplex

Upon complete construction of the new Triplex variants, immunogenicity studies took place to compare immunogenicity generated by IEfusion variant mutants and re-derived, second-generation Triplex with separated IE1 and IE2 variants, compared to first-generation Triplex. Transgenic C56BL/6 mice expressing HLA-B HLA-B*0702 (B7) or HLA-A*0201 (HHD-II) class I molecules were immunized with six second-generation Triplex constructs (A(i), A(v), B(i), B(iii), B(vii), IEfusion 4nt H363A (IGR3)::pp65 (Del3)) in addition to Triplex. Mice were vaccinated two times in 3-week intervals with the various constructs by the intraperitoneal (i.p.) route with either 2.5×10⁷ PFU (for B7 mice) or 5×10 7 PFU (for HHD-II), followed by splenocyte isolation. Human MHC-restricted T-cell responses elicited by second-generation Triplex were compared to original Triplex and an unvaccinated, naïve group as assessed by ELISpot (Table 6). For Table 6, transgenic C57BL/6 mice expressing HLA-B*0702 (B7, top) or HLA-A*0201 (HHD-II, bottom) class I molecules were immunized with various constructs expressing either IEfusion/pp65 (IEFus) or IE1/IE2/pp65. Antigen-specific T-cell responses were determined by IFN-γ Enzyme-linked immune absorbent spot (ELISpot) assay using pp65-, IE1-, and IE2-specific libraries, HLA-B*0702- or HLA-A*0201-restricted immunodominant epitopes of pp65 and IE1. DMSO was used as a negative control. Mean and standard error of the mean (SEM) values were calculated from (N) number of either HLA-B7 (top) or HHD-II (bottom) mice. SFC: cytokine-specific spot-forming cells.

TABLE 6
Human MHC-restricted T-cell responses elicited by second-generation Triplex

| | | pp65₂₆₅₋₂₇₅ | | | pp65 Library | | | IE1₃₁₆₋₃₂₄ | | | IE1 Library |
|---|---|---|---|---|---|---|---|---|---|---|---|
| | | Mean | SEM | N | Mean | SEM | N | Mean | SEM | N | Mean |
| | | HLA-B7 | | | | | | | | | |
| IFNγ | A(i) | 2223.75 | 595.6837 | 4 | 2611.25 | 736.1 | 4 | n/a | n/a | n/a | 192.8125 |
| SFCs/10\hat{ }6 | B(i) | 1730 | 235.4782 | 4 | 1690 | 316.2541 | 4 | n/a | n/a | n/a | 249.375 |
| Splenocytes | IEfus | 1323.5 | 294.2412 | 5 | 1788 | 642.6461 | 5 | n/a | n/a | n/a | 131.5 |
| | B(iii) | 1512.5 | 275.8094 | 4 | 1067.5 | 221.7121 | 4 | n/a | n/a | n/a | 143.75 |
| | A(v) | 1114 | 395.1025 | 5 | 1165.5 | 279.8911 | 5 | n/a | n/a | n/a | 234.5 |
| | B(vii) | 770.5 | 329.918 | 5 | 1060.5 | 528.679 | 5 | n/a | n/a | n/a | 99 |
| | Triplex | 2098.333 | 428.2331 | 12 | 2781.667 | 639.9631 | 12 | n/a | n/a | n/a | 103.3333 |
| | Naïve | 218.125 | 93.84926 | 4 | 320 | 76.91987 | 4 | n/a | n/a | n/a | 79.375 |
| | | HHD-II | | | | | | | | | |
| | A(i) | 1732.5 | 760.7275 | 4 | 2335 | 989.061 | 4 | 1250 | 807.3104 | 4 | 2132.5 |
| | B(i) | 355 | 64.48514 | 4 | 430 | 156.897 | 4 | 420 | 129.5505 | 4 | 795 |
| | IEfus | 1884.167 | 563.8978 | 6 | 690 | 137.3802 | 6 | 771.6667 | 177.6029 | 6 | 877.9167 |
| | B(iii) | 1132.5 | 205.1194 | 4 | 1168.125 | 133.6171 | 4 | 265.625 | 47.20716 | 4 | 850.625 |
| | A(v) | 1251 | 274.4604 | 5 | 1177.5 | 231.8203 | 5 | 822.5 | 215.1148 | 5 | 1254 |
| | B(vii) | 1906 | 828.2668 | 5 | 1752 | 370.0189 | 5 | 950.5 | 89.58725 | 5 | 1263.25 |
| | Triplex | 1884.75 | 646.1693 | 10 | 1582.25 | 420.5552 | 10 | 569 | 120.7219 | 10 | 1570.75 |
| | Naïve | 83.75 | 35.43627 | 4 | 87 | 45.24977 | 4 | 74.75 | 42.88623 | 4 | 78.25 |

| | | IE1 Library | | IE2 Library | | | DMSO | | |
|---|---|---|---|---|---|---|---|---|---|
| | | SEM | N | Mean | SEM | N | Mean | SEM | N |
| | | HLA-B7 | | | | | | | |
| IFNγ | A(i) | 25.02278 | 4 | 1111.25 | 54.04955 | 4 | 140.9375 | 21.83851 | 4 |
| SFCs/10\hat{ }6 | B(i) | 31.51347 | 4 | 1130.625 | 197.2767 | 4 | 190 | 10.05195 | 4 |
| Splenocytes | IEfus | 17.04039 | 5 | 563.5 | 100.0675 | 5 | 63 | 6.44205 | 5 |
| | B(iii) | 18.18596 | 4 | 724.375 | 148.8196 | 4 | 114.375 | 36.59142 | 4 |
| | A(v) | 60.51549 | 5 | 773.5 | 126.5815 | 5 | 137 | 30.90712 | 5 |
| | B(vii) | 4.582576 | 5 | 452.5 | 84.07214 | 5 | 42 | 6.393356 | 5 |
| | Triplex | 12.98795 | 12 | 894.1667 | 113.9371 | 12 | 51.66667 | 8.384314 | 12 |
| | Naïve | 10.32669 | 4 | 78.125 | 13.97077 | 4 | 61.25 | 9.157192 | 4 |

| | HHD-II | | | | | | | |
|---|---|---|---|---|---|---|---|---|
| A(i) | 1230.693 | 4 | 821.875 | 257.2903 | 4 | 46.875 | 9.09298 | 4 |
| B(i) | 333.4791 | 4 | 939.375 | 130.3096 | 4 | 59.375 | 14.15594 | 4 |
| IEfus | 85.47518 | 6 | 833.3333 | 131.0624 | 6 | 157.9167 | 29.61149 | 6 |
| B(iii) | 192.8794 | 4 | 1206.875 | 109.9544 | 4 | 76.875 | 3.590352 | 4 |
| A(v) | 213.9591 | 5 | 955 | 115.9418 | 5 | 69 | 8.351646 | 5 |
| B(vii) | 177.4413 | 5 | 1860.5 | 60.21524 | 5 | 81 | 7.441438 | 5 |
| Triplex | 371.4581 | 10 | 1131.25 | 115.3607 | 10 | 74.5 | 13.21195 | 10 |
| Naïve | 39.02643 | 4 | 38.125 | 10.22533 | 4 | 27 | 7.30582 | 4 |

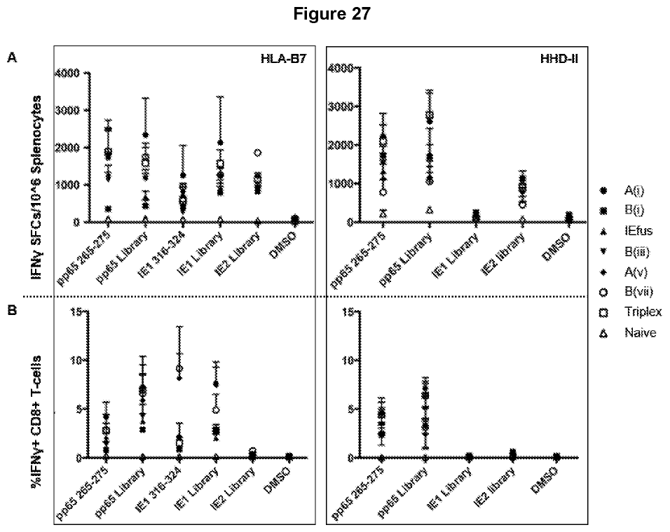

FIG. 27A shows that second-generation Triplex constructs elicited T-cell responses comparable to Triplex. However, construct B(i) seemed to underperform compared to other second-generation Triplex constructs in B7 mice (FIG. 27A, left). Construct A(i) was the most similar to Triplex in both B7 and HHD-II mice with respect to elicited T-cell responses (FIG. 27A).

T-cell stimulation from splenocytes isolated from immunized mice was also performed to evaluate antigen-specific T-cell responses, as analyzed by FACS analysis (Table 7). For Table 7, transgenic C57BL/6 mice expressing HLA-B*0702 (B7, top) or HLA-A*0201 (HHD-II, bottom) class I molecules were immunized with various constructs expressing either IEfusion/pp65 (IEFus) or IE1/IE2/pp65. Antigen-specific T-cell responses were evaluated by intracellular cytokine staining (ICS) following stimulation with pp65-, IE1-, and IE2-specific libraries or HLA-B*0702- or HLA-A*0201-restricted immunodominant epitopes of pp65 and IE1. DMSO was used as a negative control. Percentages of IFN-γ-secreting CD8+-T cells following stimulation of splenocytes from B7 or HHD-II-immunized mice with different stimuli are shown. Mean and standard error of the mean (SEM) values were calculated from (N) number of either HLA-B7 (top) or HHD-II (bottom) mice.

TABLE 7
HLA-B*0702- or HLA-A*0201-restricted CD8+ T-cell stimulation by second-generation Triplex

| | | pp65₂₆₅₋₂₇₅ | | | pp65 Library | | | IE1₃₁₆₋₃₂₄ | | | IE1 Library |
|---|---|---|---|---|---|---|---|---|---|---|---|
| | | Mean | SEM | N | Mean | SEM | N | Mean | SEM | N | Mean |
| | | HLA-B7 | | | | | | | | | |
| % IFNγ+ | A(i) | 4.54 | 1.172945 | 4 | 7.17 | 0.678085 | 4 | n/a | n/a | n/a | 0.1525 |
| CD8+ | B(i) | 2.345 | 0.27094 | 4 | 3.27 | 0.792675 | 4 | n/a | n/a | n/a | 0.23 |
| T-cells | IEfus | 4.608 | 1.559819 | 5 | 5.26 | 1.313579 | 5 | n/a | n/a | n/a | 0.1 |
| | B(iii) | 4.555 | 0.399176 | 4 | 6.11 | 2.117176 | 4 | n/a | n/a | n/a | 0.101 |
| | A(v) | 3.474 | 0.879901 | 5 | 2.408 | 1.333332 | 5 | n/a | n/a | n/a | 0.16 |
| | B(vii) | 2.44 | 1.139715 | 5 | 3.076 | 2.173167 | 5 | n/a | n/a | n/a | 0.122 |
| | Triplex | 4.409167 | 0.749607 | 12 | 6.396667 | 1.240025 | 12 | n/a | n/a | n/a | 0.076833 |
| | Naïve | 0.0575 | 0.024958 | 4 | 0.0425 | 0.004787 | 4 | n/a | n/a | n/a | 0.0525 |
| | | HHD-II | | | | | | | | | |
| | A(i) | 4.205 | 1.50755 | 4 | 7.215 | 3.182162 | 4 | 2.135 | 1.420214 | 4 | 7.6225 |
| | B(i) | 0.8 | 0.257326 | 4 | 2.89 | 0.296283 | 4 | 0.8875 | 0.161832 | 4 | 2.835 |
| | IEfus | 0.963333 | 0.211403 | 6 | 3.75 | 0.795294 | 6 | 0.87 | 0.229478 | 6 | 2.053333 |
| | B(iii) | 1.345 | 0.332979 | 4 | 4.245 | 1.22999 | 4 | 0.85 | 0.228838 | 4 | 2.37 |
| | A(v) | 1.992 | 1.056259 | 5 | 5.876 | 2.617607 | 5 | 8.16 | 2.502093 | 5 | 7.448 |
| | B(vii) | 2.834 | 1.620934 | 5 | 6.579999 | 2.948816 | 5 | 9.162001 | 4.286136 | 5 | 4.908 |
| | Triplex | 2.789 | 0.737751 | 11 | 6.963 | 1.650295 | 11 | 1.526 | 0.604693 | 11 | 2.813 |
| | Naïve | 0.21 | 0.030822 | 4 | 0.1375 | 0.03326 | 4 | 0.115 | 0.045185 | 4 | 0.1625 |

| | | IE1 Library | | IE2 Library | | | DMSO | | |
|---|---|---|---|---|---|---|---|---|---|
| | | SEM | N | Mean | SEM | N | Mean | SEM | N |
| | | HLA-B7 | | | | | | | |
| % IFNγ+ | A(i) | 0.020565 | 4 | 0.4425 | 0.118489 | 4 | 0.1325 | 0.010308 | 4 |
| CD8+ | B(i) | 0.04916 | 4 | 0.6375 | 0.087785 | 4 | 0.21 | 0.01354 | 4 |
| T-cells | IEfus | 0.007746 | 5 | 0.156 | 0.024819 | 5 | 0.044 | 0.005099 | 5 |
| | B(iii) | 0.030716 | 4 | 0.2725 | 0.029262 | 4 | 0.10775 | 0.024807 | 4 |
| | A(v) | 0.018708 | 5 | 0.262 | 0.01241 | 5 | 0.166 | 0.031718 | 5 |
| | B(vii) | 0.02245 | 5 | 0.164 | 0.023152 | 5 | 0.098 | 0.020591 | 5 |
| | Triplex | 0.014534 | 12 | 0.220667 | 0.037906 | 12 | 0.077583 | 0.017541 | 12 |
| | Naïve | 0.0075 | 4 | 0.0775 | 0.004787 | 4 | 0.0625 | 0.01315 | 4 |

| | HHD-II | | | | | | | |
|---|---|---|---|---|---|---|---|---|
| A(i) | 1.670601 | 4 | 0.185 | 0.030687 | 4 | 0.115 | 0.011902 | 4 |
| B(i) | 0.561583 | 4 | 0.3925 | 0.086446 | 4 | 0.2025 | 0.04289 | 4 |
| IEfus | 0.383855 | 6 | 0.188333 | 0.079053 | 6 | 0.096667 | 0.004216 | 6 |
| B(iii) | 0.420872 | 4 | 0.36 | 0.052281 | 4 | 0.0825 | 0.011087 | 4 |
| A(v) | 2.382548 | 5 | 0.19 | 0.040125 | 5 | 0.114 | 0.010296 | 5 |
| B(vii) | 1.599661 | 5 | 0.73 | 0.182428 | 5 | 0.16 | 0.027203 | 5 |
| Triplex | 0.612812 | 11 | 0.249 | 0.038387 | 11 | 0.091 | 0.01546 | 11 |
| Naïve | 0.01315 | 4 | 0.1225 | 0.021747 | 4 | 0.185 | 0.006455 | 4 |

FIG. 27B reiterates observations via ELISpot analysis (FIG. 27A). However, in B7 mice, B(vii) seemed to have higher T-cell stimulation than other constructs, including Triplex (FIG. 27B, left). Overall, all constructs in B7 (FIG. 27B, left) and HHD-II (FIG. 27B, right) performed as well as original Triplex.

### Example 9: Mechanism of IE2 Stability Via Zn-Finger his Mutations

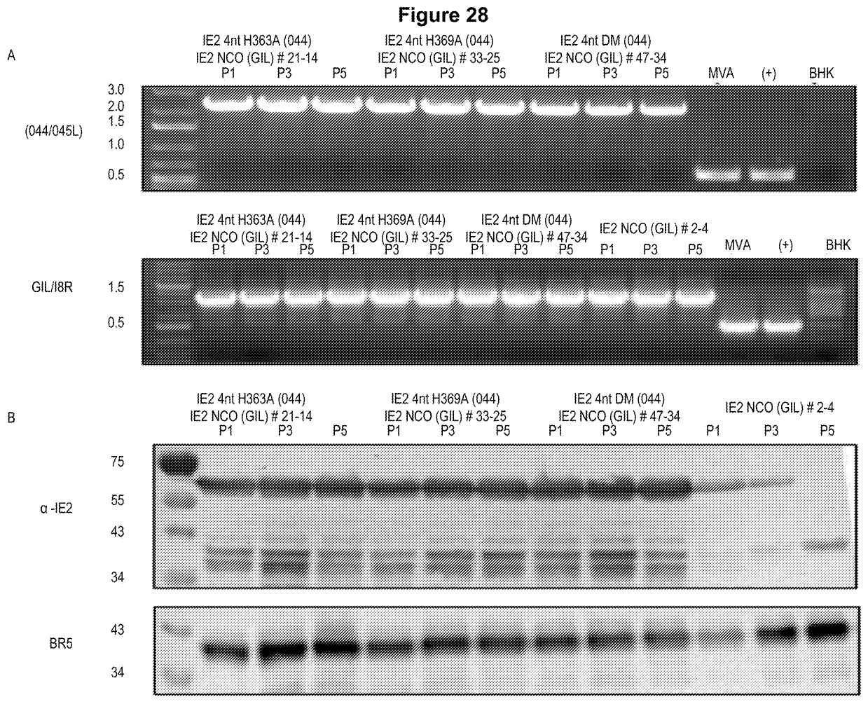

Increased stability of IE2 expressed in MVA has been observed upon mutation of one or two His residues that reside within the C-terminus of IE2 protein. To examine the effect of IE2 mutants on overall IE2 stability, an MVA was constructed to harbor two copies of IE2: IE2 NCO (wild-type) in G1L and the other in the 044/045L site harboring an IE2 mutant. MVA constructs harboring two copies of IE2 were passaged to P5 in baby hamster kidney (BHK) cells (FIG. 28). PCR analyses of both copies of IE2 show no non-specific PCR products—only products of the correct size (FIG. 28A). Western Blot analysis, on the other hand, show consistent expression of IE2 in constructs containing two IE2 copies whereas MVA:IE2 NCO (G1 L) shows a decrease in full-length IE2 expression and the emergence of a ˜40 kDa band (FIG. 28B), demonstrating a truncated product previously observed (FIG. 7). While the Western Blot showing expression of IE2 from MVAs with two IE2 copies had some degradation products, there was no concomitant increase in degradation products expected to accumulate from the passage and degradation of IE2 observed in MVA:IE2 NCO (G1L). These results could suggest a “rescue” of the IE2 instability previously observed as a result of the presence of the mutant IE2 gene insert.

### REFERENCES

The references listed below, and all references cited in the specification are hereby incorporated by reference in their entirety.
1. Wang, Z., et al., Modified H5 promoter improves stability of insert genes while maintaining immunogenicity during extended passage of genetically engineered MVA vaccines. Vaccine, 2010. 28(6): p. 1547-57.
2. Wang, Z., et al., Vaccine properties of a novel marker gene-free recombinant modified vaccinia Ankara expressing immunodominant CMV antigens pp65 and IE1. Vaccine, 2007. 25(6): p. 1132-41.
3. Wang, Z., et al., A fusion protein of HCMV IE1 exon4 and IE2 exon5 stimulates potent cellular immunity in an MVA vaccine vector. Virology, 2008. 377(2): p. 379-90.
4. Britt, W. J. and D. Auger, Identification of a 65 000 dalton virion envelope protein of human cytomegalovirus. Virus Res, 1985. 4(1): p. 31-6.
5. Meisinger-Henschel, C., et al., Introduction of the six major genomic deletions of modified vaccinia virus Ankara (MVA) into the parental vaccinia virus is not sufficient to reproduce an MVA-like phenotype in cell culture and in mice. J Virol, 2010. 84(19): p. 9907-19.
6. Dimier, J., et al., Deletion of major nonessential genomic regions in the vaccinia virus Lister strain enhances attenuation without altering vaccine efficacy in mice. J Virol, 2011. 85(10): p. 5016-26.
7. Hedengren-Olcott, M., et al., The vaccinia virus G1L putative metalloproteinase is essential for viral replication in vivo. J Virol, 2004. 78(18): p. 9947-53.
8. Bayliss, C. D. and G. L. Smith, Vaccinia virion protein I8R has both DNA and RNA helicase activities: implications for vaccinia virus transcription. J Virol, 1996. 70(2): p. 794-800.
9. Manuel, E. R., et al., Intergenic region 3 of modified vaccinia Ankara is a functional site for insert gene expression and allows for potent antigen-specific immune responses. Virology, 2010. 403(2): p. 155-62.
10. Intergenic regions as insertion sites in the genome of Modified Vaccinia Virus Ankara (MVA). P. Howley, et al., U.S. Pat. No. 7,550,157 B2 (23 Jun. 2009).
11. Cottingham, M. G., et al., Recombination-mediated genetic engineering of a bacterial artificial chromosome clone of modified vaccinia virus Ankara (MVA). PLoS One, 2008. 3(2): p. e1638.
12. Cottingham, M. G. and S. C. Gilbert, Rapid generation of markerless recombinant MVA vaccines by en passant recombineering of a self-excising bacterial artificial chromosome. J Virol Methods, 2010. 168(1-2): p. 233-6.
13. Tischer, B. K., et al., A self-excisable infectious bacterial artificial chromosome clone of varicella-zoster virus allows analysis of the essential tegument protein encoded by ORF9. J Virol, 2007. 81(23): p. 13200-8.
14. Tischer, B. K., G. A. Smith, and N. Osterrieder, En passant mutagenesis: a two step markerless red recombination system. Methods Mol Biol, 2010. 634: p. 421-30.
15. Wyatt, L. S., et al., Elucidating and minimizing the loss by recombinant vaccinia virus of human immunodeficiency virus gene expression resulting from spontaneous mutations and positive selection. J Virol, 2009. 83(14): p. 7176-84.
16. Wussow, F., et al., A vaccine based on the rhesus cytomegalovirus UL128 complex induces broadly neutralizing antibodies in rhesus macaques. J Virol, 2013. 87(3): p. 1322-32.
17. Plachter, B., et al., Analysis of proteins encoded by IE regions 1 and 2 of human cytomegalovirus using monoclonal antibodies generated against recombinant antigens. Virology, 1993. 193(2): p. 642-52.
18. White, E. A., et al., The IE2 60-kilodalton and 40-kilodalton proteins are dispensable for human cytomegalovirus replication but are required for efficient delayed early and late gene expression and production of infectious virus. J Virol, 2007. 81(6): p. 2573-83.
19. Sanders, R. L. and D. H. Spector, Human cytomegalovirus IE2 86 and IE2 40 proteins differentially regulate UL84 protein expression posttranscriptionally in the absence of other viral gene products. J Virol, 2010. 84(10): p. 5158-70.
20. Asmar, J., et al., The putative zinc finger of the human cytomegalovirus IE2 86-kilodalton protein is dispensable for DNA binding and autorepression, thereby demarcating a concise core domain in the C terminus of the protein. J Virol, 2004. 78(21): p. 11853-64.
21. Macias, M. P. and M. F. Stinski, An in vitro system for human cytomegalovirus immediate early 2 protein (IE2)-mediated site-dependent repression of transcription and direct binding of IE2 to the major immediate early promoter. Proc Natl Acad Sci USA, 1993. 90(2): p. 707-11.
22. Petrik, D. T., K. P. Schmitt, and M. F. Stinski, The autoregulatory and transactivating functions of the human cytomegalovirus IE86 protein use independent mechanisms for promoter binding. J Virol, 2007. 81(11): p. 5807-18.
23. Tang, A., et al., Functionally inactivated dominant viral antigens of human cytomegalovirus delivered in replication incompetent adenovirus type 6 vectors as vaccine candidates. Hum Vaccin Immunother, 2017. 13(12): p. 2763-2771.
24. Chiou, C. J., et al., Identification and mapping of dimerization and DNA-binding domains in the C terminus of the IE2 regulatory protein of human cytomegalovirus. J Virol, 1993. 67(10): p. 6201-14.
25. Hermiston, T. W., C. L. Malone, and M. F. Stinski, Human cytomegalovirus immediate-early two protein region involved in negative regulation of the major immediate-early promoter. J Virol, 1990. 64(7): p. 3532-6.
26. Pizzorno, M. C., et al., The functionally active IE2 immediate-early regulatory protein of human cytomegalovirus is an 80-kilodalton polypeptide that contains two distinct activator domains and a duplicated nuclear localization signal. J Virol, 1991. p. 3839-52.
27. Stenberg, R. M., et al., Promoter-specific trans activation and repression by human cytomegalovirus immediate-early proteins involves common and unique protein domains. J Virol, 1990. 64(4): p. 1556-65.
28. P. Howley, et al., Intergenic regions as novel sites for insertion of HIV DNA sequences in the genome of Modified Vaccina Virus Ankara. U.S. Pat. No. 7,501,127 B2 (10 Mar. 2009).
29. Timm, et al., Genetic stability of recombinant MVA-BNA. 2006 May 22; 24(21):4618-21. Epub 2005 Aug. 24.
30. B. Moss, et al., Recombinant Modified Vaccinia Ankara (MVA) vaccinia virus containing restructured insertion sites. U.S. Patent Application Publication No. US 2012/0263750 A1.
31. La Rosa, Longmate et al., MVA vaccine encoding CMV antigens safely induces durable expansion of CMV-specific T cells in healthy adults. Blood 2017 129(1): 114-125.
32. Sylwester, Mitchell et al., Broadly targeted human cytomegalovirus-specific CD4+ and CD8+ T cells dominate the memory compartments of exposed subjects. Journal of Experimental Medicine 2005 202(5): 673-685.
33. Nesvizhskii, A. I., Keller, A., Kolker, E. and Aebersold, R. A statistical model for identifying proteins by tandem mass spectrometry. Anal Chem. 2003: 75(17), pp. 4646-58.

1. A method of eliciting or modifying an immune response and clinical protection against CMV infection in a subject who receives a hematopoietic cell transplant (HCT), comprising administering a vaccine composition to a donor of the hematopoietic cell, wherein the vaccine composition comprises an immunologically effective amount of a recombinant modified vaccinia Ankara (rMVA) vector inserted with two or more nucleic acid sequences encoding two or more CMV antigens or antigenic portions thereof.

2. The method of claim 1, wherein the CMV antigens or antigenic fragments thereof include IE1 or IE1 exon 4 (IE1/e4), IE2 or IE2 exon 5 (IE2/e5), IEfusion of IE1/e4 and IE2/e5, and pp65.

3. The method of claim 2, wherein pp65 is co-expressed with IE1 or IE1/e4, IE2 or IE2/e5, or IEfusion.

4. The method of any one of claims 1-3, wherein two or more nucleic acid sequences are operably linked to and under the control of a single promoter.

5. The method of claim 4, wherein the promoter is the mH5 promoter.

6. The method of any one of claims 1-3, wherein each nucleic acid sequence is operably linked to and under the control of a separate promoter.

7. The method of any one of claims 1-6, wherein the donor is a human.

8. The method of any one of claims 1-7, wherein the recipient is a human.

9. The method of any one of claims 1-8, wherein the vaccine composition is administered to the donor by intramuscular administration, intradermal administration, subcutaneous, administration, intravenous administration, intranasal administration, or intraperitoneal administration.

10. The method of any one of claims 1-9, wherein the donor receives one, two, or three doses of the vaccine composition.

11. The method of any one of claims 1-10, wherein the donor receives a single dose of the vaccine composition 10-60 days prior to the start of stem cell mobilization.

12. The method of any one of claims 1-11, wherein the recipient undergoes HCT within 9 weeks of the donor's vaccination.

13. The method of any one of claims 1-12, wherein the recipient receives one or more doses of the vaccine composition after HCT.

14. The method of any one of claims 1-13, wherein the recipient receives one or more doses of the vaccine composition between day 28 and day 100 post-transplant or beyond day 100 post-transplant.

15. The method of any one of claims 1-14, wherein the HCT is HLA-matched.

16. The method of any one of claims 1-14, wherein the HCT is haploidentical or mismatched.

17. A method of treating or preventing a subject who receives a hematopoietic cell transplant (HCT) from CMV infection, comprising administering a vaccine composition to a donor of the hematopoietic cell, wherein the vaccine composition comprises an immunologically effective amount of a recombinant modified vaccinia Ankara (rMVA) vector inserted with two or more nucleic acid sequences encoding two or more CMV antigens or antigenic portions thereof.

18. The method of claim 17, wherein the CMV antigens or antigenic fragments thereof include IE1 or IE1 exon 4 (IE1/e4), IE2 or IE2 exon 5 (IE2/e5), IEfusion of IE1/e4 and IE2/e5, and pp65.

19. The method of claim 18, wherein pp65 is co-expressed with IE1 or IE1/e4, IE2 or IE2/e5, or IEfusion.

20. The method of any one of claims 17-19, wherein two or more nucleic acid sequences are operably linked to and under the control of a single promoter.

21. The method of claim 20, wherein the promoter is the mH5 promoter.

22. The method of any one of claims 17-19, wherein each nucleic acid sequence is operably linked to and under the control of a separate promoter.

23. The method of any one of claims 17-22, wherein the donor is a human.

24. The method of any one of claims 17-23, wherein the recipient is a human.

25. The method of any one of claims 17-24, wherein the vaccine composition is administered to the donor by intramuscular administration, intradermal administration, subcutaneous, administration, intravenous administration, intranasal administration, or intraperitoneal administration.

26. The method of any one of claims 17-25, wherein the donor receives one, two, or three doses of the vaccine composition.

27. The method of any one of claims 17-26, wherein the donor receives a single dose of the vaccine composition 10-60 days prior to the start of stem cell mobilization.

28. The method of any one of claims 17-27, wherein the recipient undergoes HCT within 9 weeks of the donor's vaccination.

29. The method of any one of claims 17-28, wherein the recipient receives one or more doses of the vaccine composition after HCT.

30. The method of any one of claims 17-29, wherein the recipient receives one or more doses of the vaccine composition between day 28 and day 100 post-transplant or beyond day 100 post-transplant.

31. The method of any one of claims 17-30, wherein the HCT is HLA-matched.

32. The method of any one of claims 17-30, wherein the HCT is haploidentical or mismatched.
